(19) United States
(12) Patent Application Publication
(10) Pub. No.: US 2021/0023041 A1
(43) Pub. Date: Jan. 28, 2021

(54) FORMULATIONS AND COMPOSITIONS OF DOCETAXEL

(71) Applicant: Zhuhai Beihai Biotech Co., Ltd., Zhuhai, Guangdong (CN)

(72) Inventor: Qun Sun, Princeton, NJ (US)

(21) Appl. No.: 16/982,772

(22) PCT Filed: Apr. 11, 2019
(86) PCT No.: PCT/US2019/026965

§ 371 Date: Sep. 21, 2020

Related U.S. Application Data
(60) Provisional application No. 62/827,561, filed on Apr. 1, 2019.
(60) Provisional application No. 62/793,048, filed on Jan. 16, 2019.
(60) Provisional application No. 62/656,043, filed on Apr. 11, 2018.

(51) Int. Cl. A61K 31/337; A61K 9/08; A61K 47/42; A61K 47/18; A61K 47/26

(52) U.S. Cl. CPC A61K 31/337; A61K 9/08; A61K 47/26; A61K 47/183; A61K 47/42

(57) ABSTRACT

This document relates to a composition comprising Docetaxel, human serum albumin, and one or more amino acids selected from aspartic acid, glutamic acid, and cysteine, wherein the human serum albumin and the Docetaxel in the composition have a ratio by weight of no less than 60:1. This document also relates to a composition comprising Docetaxel, human serum albumin, one or more amino acids selected from aspartic acid, glutamic acid, and cysteine, and a sugar alcohol or a sugar, wherein the human serum albumin and the Docetaxel in the composition have a ratio by weight of no less than 60:1.

### CLAIM OF PRIORITY

This application claims the benefit of U.S. provisional application Ser. Nos. 62/656,043, filed Apr. 11, 2018; 62/793,048, filed Jan. 16, 2019; and 62/827,561, filed Apr. 1, 2019.

### TECHNICAL FIELD

This document relates to formulations and compositions for the treatment of proliferative diseases, more particularly to formulations and compositions comprising Docetaxel and human serum albumin, and more particularly to formulations and compositions comprising Docetaxel and human serum albumin with improved stability.

### BACKGROUND

Many drugs for parenteral use are insoluble in water, and are thus formulated with solubilizing agents, surfactants, solvents, and/or emulsifiers that are irritating, allergenic, or toxic when administered to patients. See, e.g., Briggs et al., Anesthesis 37, 1099 (1982), and Waugh et al., Am. J. Hosp. Pharmacists, 48, 1520 (1991)). Further, many of these drugs, especially those administered intravenously, cause undesirable side effects such as venous irritation, phlebitis, burning and pain on injection, venous thrombosis, extravasation, and other administration related side effects. Additionally, often free drugs present in formulations induce pain or irritation upon administration.

Taxanes play an important role in the treatment of various solid tumors. As a second-generation semi-synthetic taxane derivative, Docetaxel is about twice as potent as paclitaxel in inhibiting microtubule depolymerization, and has the unique ability to alter certain classes of microtubules, which differs from most spindle poisons currently used in clinic. However, Docetaxel has very poor water solubility. The clinical intravenous administration of commercially available Docetaxel (Taxotere®) is formulated in a highly concentrated solution containing 40 mg Docetaxel and 1040 mg Polysorbate 80 per mL. This concentrated solution must be carefully diluted with solvent containing 13% ethanol in saline before administration, and must be used within 4 hours due to its limited stability. These attributes limit the administration of Docetaxel. Further, it has been reported that docetaxel administration is associated with the occurrence of unpredictable (acute) hypersensitivity reactions and cumulative fluid retention. See, e.g., Trudeau M E et al., J Clin Oncol 1996; 14:422-8, Piccart M J et al., J Natl Cancer Inst 1995; 87:676-81, Bruno R et al., J Clin Oncol 1998; 16:187-96. These side-effects have been attributed, in part, to the presence of polysorbate 80.

US 2005/0282734 describes complexes of paclitaxel and albumin. Successful formulations described in this document require acidic pH. WO 2014/121033 describes complexes of camptothecin and albumin. US 2012/0076862 describes nanoparticles of taxane and albumin. US 2010/0076008 describes paclitaxel non-covalently bound to HSA. WO 2016/187147 describes complexes and compositions of docetaxel and HSA. WO 2018/081520 describes neutral pH compositions of docetaxel and HSA. WO 2018/204386 describes formulations and compositions of docetaxel and HSA.

Docetaxel degrades in various conditions. In basic, neutral, or strong acidic environment, one of major degradation routes of docetaxel is epimerization of C7 position of hydroxy group to produce 7-Epi-docetaxel. The formation of 7-Epi-docetaxel by the epimerization has been implicated in loss of potency of the drug and as well as in the development of resistance in the tumour cells. See, e.g., Bournique et al., Drug Metabolism and Disposition 30, 1149-1152 (2002), Czejka et al., Journal of Analytical Oncology 3, 73-78 (2014), and Mohsin et al., Drug Testing and Analysis, 6, 1076-1084 (2014). There is a need to prevent and reduce the formation of 7-Epi-docetaxel in the preparation of new docetaxel formulations and in the drug storage process.

WO 2016/155595 describes that adding amino acids, such as arginine, into a nanoparticle formulation of docetaxel and albumin reduces the formation of 7-epi-docetaxel in the formulation.

Accordingly, there is a need in the art for more stable formulations of Docetaxel. The compositions and methods described in the present application help meet this need.

### SUMMARY

Provided herein is a composition comprising Docetaxel, human serum albumin, and one or more amino acids selected from aspartic acid, glutamic acid, and cysteine, wherein the human serum albumin and the Docetaxel in the composition have a ratio by weight of no less than 60:1. In some embodiments, the amino acid and the Docetaxel in the composition have a ratio by weight of no less than 0.2:1. In some embodiments, the pH of a composition is neutral (e.g., pH of the composition is from about 5 to about 8, from about 5.5 to about 7.5, or from about 5.5 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the human serum albumin and the Docetaxel in the composition have a ratio by weight of no less than about 70:1. In some embodiments, the Docetaxel and the human serum albumin in the composition are in a ratio by weight from about 1:70 to about 1:250. In some embodiments, the Docetaxel and the human serum albumin in the composition are in a ratio by weight from about 1:80 to about 1:200. In some embodiments, the Docetaxel and the human serum albumin in the composition are in a ratio by weight from about 1:90 to about 1:200. In some embodiments, the Docetaxel and the human serum albumin in the composition are in a ratio by weight from about 1:95 to about 1:200. In some embodiments, the Docetaxel and the human serum albumin in the composition are in a ratio by weight from about 1:100 to about 1:200. In some embodiments, the Docetaxel and the human serum albumin in the composition are in a ratio by weight from about 1:80 to about 1:175. In some embodiments, the Docetaxel and the human serum albumin in the composition are in a ratio by weight from about 1:90 to about 1:175. In some embodiments, the Docetaxel and the human serum albumin in the composition are in a ratio by weight from about 1:95 to about 1:175. In some embodiments, the Docetaxel and the human serum albumin in the composition are in a ratio by weight from about 1:80 to about 1:150. In some embodiments, the Docetaxel and the human serum albumin in the composition are in a ratio by weight from about 1:90 to about 1:150. In some embodiments, the Docetaxel and the human serum albumin in the composition are in a ratio by weight from about 1:95 to about 1:150. In some embodiments, the Docetaxel and the human serum albumin in the composition are in a ratio by weight from about 1:100 to about 1:150. In some embodiments, the Docetaxel and the human serum albumin in the composition are in a ratio by weight from about 1:80 to about 1:140. In some embodiments, the Docetaxel and the human serum albumin in the composition are in a ratio by weight from about 1:85 to about 1:140. In some embodiments, the Docetaxel and the human serum albumin in the composition are in a ratio by weight from about 1:90 to about 1:140. In some embodiments, the Docetaxel and the human serum albumin in the composition are in a ratio by weight from about 1:95 to about 1:140. In some embodiments, the Docetaxel and the human serum albumin in the composition are in a ratio by weight from about 1:80 to about 1:130. In some embodiments, the Docetaxel and the human serum albumin in the composition are in a ratio by weight from about 1:90 to about 1:130. In some embodiments, the Docetaxel and the human serum albumin in the composition are in a ratio by weight from about 1:95 to about 1:130. In some embodiments, the Docetaxel and the human serum albumin in the composition are in a ratio by weight from about 1:100 to about 1:130. In some embodiments, the Docetaxel and the human serum albumin in the composition are in a ratio by weight from about 1:85 to about 1:125. In some embodiments, the Docetaxel and the human serum albumin in the composition are in a ratio by weight from about 1:90 to about 1:125. In some embodiments, the Docetaxel and the human serum albumin in the composition are in a ratio by weight from about 1:95 to about 1:125. In some embodiments, the Docetaxel and the human serum albumin in the composition are in a ratio by weight from about 1:85 to about 1:120. In some embodiments, the Docetaxel and the human serum albumin in the composition are in a ratio by weight from about 1:90 to about 1:120. In some embodiments, the Docetaxel and the human serum albumin in the composition are in a ratio by weight from about 1:90 to about 1:110. In some embodiments, the Docetaxel and the human serum albumin in the composition are in a ratio by weight from about 1:95 to about 1:105. In some embodiments, the Docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1: 140, about 1:145, about 1:150, about 1:155, about 1:160, about 1:170, about 1:180, about 1:190, about 1:200, about 1:210, about 1:220, or about 1:250. In some embodiments, the Docetaxel and the human serum albumin in the composition are in a ratio by weight of about 1:90. In some embodiments, the Docetaxel and the human serum albumin in the composition are in a ratio by weight of about 1:95. In some embodiments, the Docetaxel and the human serum albumin in the composition are in a ratio by weight of about 1:100. In some embodiments, the Docetaxel and the human serum albumin in the composition are in a ratio by weight of about 1:105. In some embodiments, the Docetaxel and the human serum albumin in the composition are in a ratio by weight of about 1:110. In some embodiments, the Docetaxel and the human serum albumin in the composition are in a ratio by weight of about 1:115. In some embodiments, the Docetaxel and the human serum albumin in the composition are in a ratio by weight of about 1:120.

In some embodiments, the composition comprises one amino acid. In some embodiments, the amino acid in the composition is aspartic acid. In some embodiments, the composition comprises aspartic acid. In some embodiments, the amino acid in the composition is glutamic acid. In some embodiments, the composition comprises glutamic acid. In some embodiments, the composition comprises one amino acid, wherein the amino acid in the composition is aspartic acid. In some embodiments, the composition comprises one amino acid, wherein the amino acid in the composition is glutamic acid. In some embodiments, the composition comprises two amino acids. In some embodiments, the composition comprises two amino acids, wherein at least one amino acid in the composition is aspartic acid or glutamic acid. In some embodiments, the composition comprises two amino acids, wherein at least one amino acid in the composition is aspartic acid. In some embodiments, the composition comprises two amino acids, wherein at least one amino acid in the composition is glutamic acid. In some embodiments, the amino acid and the Docetaxel in the composition have a ratio by weight of no less than about 0.5:1. In some embodiments, the amino acid and the Docetaxel in the composition have a ratio by weight of no less than about 1:1. In some embodiments, the amino acid and the Docetaxel in the composition have a ratio by weight of no less than about 2:1. In some embodiments, the amino acid and the Docetaxel in the composition have a ratio by weight of about 2:1. In some embodiments, the amino acid and the Docetaxel in the composition have a ratio by weight of 2:1.

In some embodiments, the composition further comprises a sugar alcohol or a sugar. In some embodiments, the composition further comprises a sugar alcohol. In some embodiments, the sugar alcohol in the composition is selected from mannitol, sorbitol, inositol, and xylitol. In some embodiments, the sugar alcohol in the composition is mannitol. In some embodiments, the sugar alcohol in the composition is sorbitol. In some embodiments, the sugar alcohol and the Docetaxel in the composition have a ratio by weight of no less than about 5:1. In some embodiments, the sugar alcohol and the Docetaxel in the composition have a ratio by weight of no less than about 10:1. In some embodiments, the sugar alcohol and the Docetaxel in the composition have a ratio by weight of no less than about 15:1. In some embodiments, the sugar alcohol and the Docetaxel in the composition have a ratio by weight of no less than about 20:1. In some embodiments, the sugar alcohol and the Docetaxel in the composition have a ratio by weight of no less than about 25:1. In some embodiments, the sugar alcohol and the Docetaxel in the composition have a ratio by weight of no less than about 50:1. In some embodiments, the sugar alcohol and the Docetaxel in the composition have a ratio by weight of no less than about 75:1. In some embodiments, the sugar alcohol and the Docetaxel in the composition have a ratio by weight of no less than about 100:1. In some embodiments, the sugar alcohol and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar alcohol and the Docetaxel in the composition have a ratio by weight of about 20:1. In some embodiments, mannitol and the Docetaxel in the composition have a ratio by weight of about 20:1.

In some embodiments, the composition further comprises a sugar. In some embodiments, the sugar in the composition is selected from glucose, sucrose, lactose, maltose, trehalose, fructose, hexose, and raffinose. In some embodiments, the sugar in the composition is glucose. In some embodiments, the sugar in the composition is lactose. In some embodiments, the sugar and the Docetaxel in the composition have a ratio by weight of no less than about 5:1. In some embodiments, the sugar and the Docetaxel in the composition have a ratio by weight of no less than about 10:1. In some embodiments, the sugar and the Docetaxel in the composition have a ratio by weight of no less than about 15:1. In some embodiments, the sugar and the Docetaxel in the composition have a ratio by weight of no less than about 20:1. In some embodiments, the sugar and the Docetaxel in the composition have a ratio by weight of no less than about 25:1. In some embodiments, the sugar and the Docetaxel in the composition have a ratio by weight of no less than about 50:1. In some embodiments, the sugar and the Docetaxel in the composition have a ratio by weight of no less than about 75:1. In some embodiments, the sugar and the Docetaxel in the composition have a ratio by weight of no less than about 100:1. In some embodiments, the sugar and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar and the Docetaxel in the composition have a ratio by weight of about 20:1. In some embodiments, glucose and the Docetaxel in the composition have a ratio by weight of about 20:1.

In some embodiments, the human serum albumin is a native human serum albumin. In some embodiments, the human serum albumin is a native human serum albumin obtained from pools of human plasma. In some embodiments, the human serum albumin is a recombinant human serum albumin. In some embodiments, the human serum albumin is a fatty acid free human serum albumin. In some embodiments, the human serum albumin is essentially fatty acid free.

In some embodiments, the composition is a solid formulation. In some embodiments, the composition is an aqueous formulation. In some embodiments, the aqueous formulation is substantially free of solvent other than water. In some embodiments, the aqueous formulation is a clear aqueous solution. In some embodiments, the pH of the solid formulation or the aqueous formulation is neutral (e.g., pH of the composition is from about 5 to about 8, from about 5.5 to about 7.5, or from about 5.5 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

Also, provided herein is a pharmaceutical composition comprising Docetaxel, human serum albumin, and one or more amino acids selected from aspartic acid, glutamic acid, or cysteine as described herein, and a pharmaceutically acceptable carrier. In some embodiments, provided herein is a pharmaceutical composition comprising Docetaxel, human serum albumin, one or more amino acids selected from aspartic acid, glutamic acid, or cysteine, and a sugar alcohol or a sugar as described herein, and a pharmaceutically acceptable carrier.

Also, provided herein is a method of treating cancer comprising the step of administering to a subject in need thereof a therapeutically effective amount of a pharmaceutical composition comprising the composition comprising Docetaxel, human serum albumin, and one or more amino acids selected from aspartic acid, glutamic acid, or cysteine as described herein, and a pharmaceutically acceptable carrier. In some embodiments, provided herein is a method of treating cancer comprising the step of administering to a subject in need thereof a therapeutically effective amount of a pharmaceutical composition comprising the composition comprising Docetaxel, human serum albumin, one or more amino acids selected from aspartic acid, glutamic acid, or cysteine, and a sugar alcohol or a sugar as described herein, and a pharmaceutically acceptable carrier.

In some embodiments, the cancer is a solid tumor cancer. In some embodiments, the cancer is selected from the group consisting of breast cancer, non-small cell lung cancer, prostate cancer, gastric cancer, head and neck cancer, ovarian cancer, pancreatic cancer, and Kaposi's sarcoma. In some embodiments, the cancer is a breast cancer. In some embodiments, the cancer is a non-small cell lung cancer. In some embodiments, the cancer is a prostate cancer. In some embodiments, the cancer is a gastric cancer. In some embodiments, the cancer is a head and neck cancer. In some embodiments the cancer is an ovarian cancer. In some embodiments, the cancer is a pancreatic cancer. In some embodiments, the cancer is a Kaposi's sarcoma.

Also, provided herein is a composition comprising Docetaxel, human serum albumin, and a sugar alcohol or a sugar, wherein the human serum albumin and the Docetaxel in the composition have a ratio by weight of no less than 60:1. In some embodiments, the pH of a composition is neutral (e.g., pH of the composition is from about 5 to about 8, from about 5.5 to about 7.5, or from about 5.5 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the human serum albumin and the Docetaxel in the composition have a ratio by weight of no less than about 70:1. In some embodiments, the Docetaxel and the human serum albumin in the composition are in a ratio by weight from about 1:70 to about 1:250. In some embodiments, the Docetaxel and the human serum albumin in the composition are in a ratio by weight from about 1:80 to about 1:200. In some embodiments, the Docetaxel and the human serum albumin in the composition are in a ratio by weight from about 1:90 to about 1:200. In some embodiments, the Docetaxel and the human serum albumin in the composition are in a ratio by weight from about 1:95 to about 1:200. In some embodiments, the Docetaxel and the human serum albumin in the composition are in a ratio by weight from about 1:100 to about 1:200. In some embodiments, the Docetaxel and the human serum albumin in the composition are in a ratio by weight from about 1:80 to about 1:175. In some embodiments, the Docetaxel and the human serum albumin in the composition are in a ratio by weight from about 1:90 to about 1:175. In some embodiments, the Docetaxel and the human serum albumin in the composition are in a ratio by weight from about 1:95 to about 1:175. In some embodiments, the Docetaxel and the human serum albumin in the composition are in a ratio by weight from about 1:80 to about 1:150. In some embodiments, the Docetaxel and the human serum albumin in the composition are in a ratio by weight from about 1:90 to about 1:150. In some embodiments, the Docetaxel and the human serum albumin in the composition are in a ratio by weight from about 1:95 to about 1:150. In some embodiments, the Docetaxel and the human serum albumin in the composition are in a ratio by weight from about 1:100 to about 1:150. In some embodiments, the Docetaxel and the human serum albumin in the composition are in a ratio by weight from about 1:80 to about 1:140. In some embodiments, the Docetaxel and the human serum albumin in the composition are in a ratio by weight from about 1:85 to about 1:140. In some embodiments, the Docetaxel and the human serum albumin in the composition are in a ratio by weight from about 1:90 to about 1:140. In some embodiments, the Docetaxel and the human serum albumin in the composition are in a ratio by weight from about 1:95 to about 1:140. In some embodiments, the Docetaxel and the human serum albumin in the composition are in a ratio by weight from about 1:80 to about 1:130. In some embodiments, the Docetaxel and the human serum albumin in the composition are in a ratio by weight from about 1:90 to about 1:130. In some embodiments, the Docetaxel and the human serum albumin in the composition are in a ratio by weight from about 1:95 to about 1:130. In some embodiments, the Docetaxel and the human serum albumin in the composition are in a ratio by weight from about 1:100 to about 1:130. In some embodiments, the Docetaxel and the human serum albumin in the composition are in a ratio by weight from about 1:85 to about 1:125. In some embodiments, the Docetaxel and the human serum albumin in the composition are in a ratio by weight from about 1:90 to about 1:125. In some embodiments, the Docetaxel and the human serum albumin in the composition are in a ratio by weight from about 1:95 to about 1:125. In some embodiments, the Docetaxel and the human serum albumin in the composition are in a ratio by weight from about 1:85 to about 1:120. In some embodiments, the Docetaxel and the human serum albumin in the composition are in a ratio by weight from about 1:90 to about 1:120. In some embodiments, the Docetaxel and the human serum albumin in the composition are in a ratio by weight from about 1:90 to about 1:110. In some embodiments, the Docetaxel and the human serum albumin in the composition are in a ratio by weight from about 1:95 to about 1:105. In some embodiments, the Docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1: 140, about 1:145, about 1:150, about 1:155, about 1:160, about 1:170, about 1:180, about 1:190, about 1:200, about 1:210, about 1:220, or about 1:250. In some embodiments, the Docetaxel and the human serum albumin in the composition are in a ratio by weight of about 1:90. In some embodiments, the Docetaxel and the human serum albumin in the composition are in a ratio by weight of about 1:95. In some embodiments, the Docetaxel and the human serum albumin in the composition are in a ratio by weight of about 1:100. In some embodiments, the Docetaxel and the human serum albumin in the composition are in a ratio by weight of about 1:105. In some embodiments, the Docetaxel and the human serum albumin in the composition are in a ratio by weight of about 1:110. In some embodiments, the Docetaxel and the human serum albumin in the composition are in a ratio by weight of about 1:115. In some embodiments, the Docetaxel and the human serum albumin in the composition are in a ratio by weight of about 1:120.

In some embodiments, the composition comprises a sugar alcohol. In some embodiments, the sugar alcohol in the composition is selected from mannitol, sorbitol, inositol, and xylitol. In some embodiments, the sugar alcohol in the composition is mannitol. In some embodiments, the sugar alcohol in the composition is sorbitol. In some embodiments, the sugar alcohol and the Docetaxel in the composition have a ratio by weight of no less than about 5:1. In some embodiments, the sugar alcohol and the Docetaxel in the composition have a ratio by weight of no less than about 10:1. In some embodiments, the sugar alcohol and the Docetaxel in the composition have a ratio by weight of no less than about 15:1. In some embodiments, the sugar alcohol and the Docetaxel in the composition have a ratio by weight of no less than about 20:1. In some embodiments, the sugar alcohol and the Docetaxel in the composition have a ratio by weight of no less than about 25:1. In some embodiments, the sugar alcohol and the Docetaxel in the composition have a ratio by weight of no less than about 50:1. In some embodiments, the sugar alcohol and the Docetaxel in the composition have a ratio by weight of no less than about 75:1. In some embodiments, the sugar alcohol and the Docetaxel in the composition have a ratio by weight of no less than about 100:1. In some embodiments, the sugar alcohol and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar alcohol and the Docetaxel in the composition have a ratio by weight of about 20:1. In some embodiments, mannitol and the Docetaxel in the composition have a ratio by weight of about 20:1.

In some embodiments, the composition comprises a sugar. In some embodiments, the sugar in the composition is selected from glucose, sucrose, lactose, maltose, trehalose, fructose, hexose, and raffinose. In some embodiments, the sugar in the composition is glucose. In some embodiments, the sugar in the composition is lactose. In some embodiments, the sugar and the Docetaxel in the composition have a ratio by weight of no less than about 5:1. In some embodiments, the sugar and the Docetaxel in the composition have a ratio by weight of no less than about 10:1. In some embodiments, the sugar and the Docetaxel in the composition have a ratio by weight of no less than about 15:1. In some embodiments, the sugar and the Docetaxel in the composition have a ratio by weight of no less than about 20:1. In some embodiments, the sugar and the Docetaxel in the composition have a ratio by weight of no less than about 25:1. In some embodiments, the sugar and the Docetaxel in the composition have a ratio by weight of no less than about 50:1. In some embodiments, the sugar and the Docetaxel in the composition have a ratio by weight of no less than about 75:1. In some embodiments, the sugar and the Docetaxel in the composition have a ratio by weight of no less than about 100:1. In some embodiments, the sugar and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar and the Docetaxel in the composition have a ratio by weight of about 20:1. In some embodiments, glucose and the Docetaxel in the composition have a ratio by weight of about 20:1.

In some embodiments, the human serum albumin is a native human serum albumin. In some embodiments, the human serum albumin is a native human serum albumin obtained from pools of human plasma. In some embodiments, the human serum albumin is a recombinant human serum albumin. In some embodiments, the human serum albumin is a fatty acid free human serum albumin. In some embodiments, the human serum albumin is essentially fatty acid free.

Also, provided herein is a composition comprising Docetaxel and human serum albumin, wherein 7-Epi-docetaxel and the Docetaxel in the composition have a ratio by weight of no more than 1:100, and wherein the human serum albumin and the Docetaxel in the composition have a ratio by weight of no less than 60:1. In some embodiments, the pH of a composition is neutral (e.g., pH of the composition is from about 5 to about 8, from about 5.5 to about 7.5, or from about 5.5 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, 7-Epi-docetaxel and the Docetaxel in the composition have a ratio by weight of no more than 1:200. In some embodiments, 7-Epi-docetaxel and the Docetaxel in the composition have a ratio by weight of no more than 1:300. In some embodiments, 7-Epi-docetaxel and the Docetaxel in the composition have a ratio by weight of no more than 1:500. In some embodiments, 7-Epi-docetaxel and the Docetaxel in the composition have a ratio by weight of no more than 1:1000. In some embodiments, 7-Epi-docetaxel and the Docetaxel in the composition have a ratio by weight of no more than 1:2000.

In some embodiments, the human serum albumin and the Docetaxel in the composition have a ratio by weight of no less than 70:1. In some embodiments, the Docetaxel and the human serum albumin in the composition are in a ratio by weight from about 1:70 to about 1:250. In some embodiments, the Docetaxel and the human serum albumin in the composition are in a ratio by weight from about 1:80 to about 1:200. In some embodiments, the Docetaxel and the human serum albumin in the composition are in a ratio by weight from about 1:90 to about 1:200. In some embodiments, the Docetaxel and the human serum albumin in the composition are in a ratio by weight from about 1:95 to about 1:200. In some embodiments, the Docetaxel and the human serum albumin in the composition are in a ratio by weight from about 1:100 to about 1:200. In some embodiments, the Docetaxel and the human serum albumin in the composition are in a ratio by weight from about 1:80 to about 1:175. In some embodiments, the Docetaxel and the human serum albumin in the composition are in a ratio by weight from about 1:90 to about 1:175. In some embodiments, the Docetaxel and the human serum albumin in the composition are in a ratio by weight from about 1:95 to about 1:175. In some embodiments, the Docetaxel and the human serum albumin in the composition are in a ratio by weight from about 1:80 to about 1:150. In some embodiments, the Docetaxel and the human serum albumin in the composition are in a ratio by weight from about 1:90 to about 1:150. In some embodiments, the Docetaxel and the human serum albumin in the composition are in a ratio by weight from about 1:95 to about 1:150. In some embodiments, the Docetaxel and the human serum albumin in the composition are in a ratio by weight from about 1:100 to about 1:150. In some embodiments, the Docetaxel and the human serum albumin in the composition are in a ratio by weight from about 1:80 to about 1:140. In some embodiments, the Docetaxel and the human serum albumin in the composition are in a ratio by weight from about 1:85 to about 1:140. In some embodiments, the Docetaxel and the human serum albumin in the composition are in a ratio by weight from about 1:90 to about 1:140. In some embodiments, the Docetaxel and the human serum albumin in the composition are in a ratio by weight from about 1:95 to about 1:140. In some embodiments, the Docetaxel and the human serum albumin in the composition are in a ratio by weight from about 1:80 to about 1:130. In some embodiments, the Docetaxel and the human serum albumin in the composition are in a ratio by weight from about 1:90 to about 1:130. In some embodiments, the Docetaxel and the human serum albumin in the composition are in a ratio by weight from about 1:95 to about 1:130. In some embodiments, the Docetaxel and the human serum albumin in the composition are in a ratio by weight from about 1:100 to about 1:130. In some embodiments, the Docetaxel and the human serum albumin in the composition are in a ratio by weight from about 1:85 to about 1:125. In some embodiments, the Docetaxel and the human serum albumin in the composition are in a ratio by weight from about 1:90 to about 1:125. In some embodiments, the Docetaxel and the human serum albumin in the composition are in a ratio by weight from about 1:95 to about 1:125. In some embodiments, the Docetaxel and the human serum albumin in the composition are in a ratio by weight from about 1:85 to about 1:120. In some embodiments, the Docetaxel and the human serum albumin in the composition are in a ratio by weight from about 1:90 to about 1:120. In some embodiments, the Docetaxel and the human serum albumin in the composition are in a ratio by weight from about 1:90 to about 1:110. In some embodiments, the Docetaxel and the human serum albumin in the composition are in a ratio by weight from about 1:95 to about 1:105. In some embodiments, the Docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1: 140, about 1:145, about 1:150, about 1:155, about 1:160, about 1:170, about 1:180, about 1:190, about 1: 200, about 1:210, about 1:220, or about 1: 250. In some embodiments, the Docetaxel and the human serum albumin in the composition are in a ratio by weight of about 1:90. In some embodiments, the Docetaxel and the human serum albumin in the composition are in a ratio by weight of about 1:95. In some embodiments, the Docetaxel and the human serum albumin in the composition are in a ratio by weight of about 1:100. In some embodiments, the Docetaxel and the human serum albumin in the composition are in a ratio by weight of about 1:105. In some embodiments, the Docetaxel and the human serum albumin in the composition are in a ratio by weight of about 1:110. In some embodiments, the Docetaxel and the human serum albumin in the composition are in a ratio by weight of about 1:115. In some embodiments, the Docetaxel and the human serum albumin in the composition are in a ratio by weight of about 1:120.

In some embodiments, the human serum albumin is a native human serum albumin. In some embodiments, the human serum albumin is a native human serum albumin obtained from pools of human plasma. In some embodiments, the human serum albumin is a recombinant human serum albumin. In some embodiments, the human serum albumin is a fatty acid free human serum albumin. In some embodiments, the human serum albumin is essentially fatty acid free.

In some embodiments, the composition further comprises a sugar alcohol or a sugar. In some embodiments, the composition further comprises a sugar alcohol. In some embodiments, the sugar alcohol in the composition is selected from mannitol, sorbitol, inositol, and xylitol. In some embodiments, the sugar alcohol in the composition is mannitol. In some embodiments, the sugar alcohol in the composition is sorbitol. In some embodiments, the sugar alcohol and the Docetaxel in the composition have a ratio by weight of no less than about 5:1. In some embodiments, the sugar alcohol and the Docetaxel in the composition have a ratio by weight of no less than about 10:1. In some embodiments, the sugar alcohol and the Docetaxel in the composition have a ratio by weight of no less than about 15:1. In some embodiments, the sugar alcohol and the Docetaxel in the composition have a ratio by weight of no less than about 20:1. In some embodiments, the sugar alcohol and the Docetaxel in the composition have a ratio by weight of no less than about 25:1. In some embodiments, the sugar alcohol and the Docetaxel in the composition have a ratio by weight of no less than about 50:1. In some embodiments, the sugar alcohol and the Docetaxel in the composition have a ratio by weight of no less than about 75:1. In some embodiments, the sugar alcohol and the Docetaxel in the composition have a ratio by weight of no less than about 100:1. In some embodiments, the sugar alcohol and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar alcohol and the Docetaxel in the composition have a ratio by weight of about 20:1. In some embodiments, mannitol and the Docetaxel in the composition have a ratio by weight of about 20:1.

In some embodiments, the composition further comprises a sugar. In some embodiments, the sugar in the composition is selected from glucose, sucrose, lactose, maltose, trehalose, fructose, hexose, and raffinose. In some embodiments, the sugar in the composition is glucose. In some embodiments, the sugar in the composition is lactose. In some embodiments, the sugar and the Docetaxel in the composition have a ratio by weight of no less than about 5:1. In some embodiments, the sugar and the Docetaxel in the composition have a ratio by weight of no less than about 10:1. In some embodiments, the sugar and the Docetaxel in the composition have a ratio by weight of no less than about 15:1. In some embodiments, the sugar and the Docetaxel in the composition have a ratio by weight of no less than about 20:1. In some embodiments, the sugar and the Docetaxel in the composition have a ratio by weight of no less than about 25:1. In some embodiments, the sugar and the Docetaxel in the composition have a ratio by weight of no less than about 50:1. In some embodiments, the sugar and the Docetaxel in the composition have a ratio by weight of no less than about 75:1. In some embodiments, the sugar and the Docetaxel in the composition have a ratio by weight of no less than about 100:1. In some embodiments, the sugar and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar and the Docetaxel in the composition have a ratio by weight of about 20:1. In some embodiments, glucose and the Docetaxel in the composition have a ratio by weight of about 20:1.

Unless otherwise defined, all technical and scientific terms used herein have the same meaning as commonly understood by one of ordinary skill in the art to which this disclosure belongs. Methods and materials are described herein for use in the present disclosure; other suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

### DETAILED DESCRIPTION

Provided herein is a composition comprising Docetaxel, human serum albumin, and one or more amino acids selected from aspartic acid, glutamic acid, and cysteine, wherein the human serum albumin and the Docetaxel in the composition have a ratio by weight of no less than 60:1. In some embodiments, the amino acid and the Docetaxel in the composition have a ratio by weight of no less than 0.2:1. In some embodiments, the pH of a composition is neutral (e.g., pH of the composition is from about 5 to about 8, from about 5.5 to about 7.5, from about 5.5 to about 6.5, or from about 5.5 to about 7, or the pH of the composition is about 5, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

WO 2016/155595 describes adding amino acids, such as arginine, into a nanoparticle formulation of docetaxel and albumin decreases the formation of 7-Epi-docetaxel in the formulation. We surprisingly find that adding arginine actually increases the formation of 7-Epi-docetaxel in our formulation of docetaxel and human serum albumin, while adding aspartic acid or glutamic acid decreases the formation of 7-Epi-docetaxel. See the experiment results described here in the example section.

In some embodiments, 7-Epi-docetaxel and the Docetaxel in the composition have a ratio by weight of no more than 1:100. In some embodiments, 7-Epi-docetaxel and the Docetaxel in the composition have a ratio by weight of no more than 1:200. In some embodiments, 7-Epi-docetaxel and the Docetaxel in the composition have a ratio by weight of no more than 1:300. In some embodiments, 7-Epi-docetaxel and the Docetaxel in the composition have a ratio by weight of no more than 1:500. In some embodiments, 7-Epi-docetaxel and the Docetaxel in the composition have a ratio by weight of no more than 1:1000. In some embodiments, 7-Epi-docetaxel and the Docetaxel in the composition have a ratio by weight of no more than 1:2000.

In some embodiments, the human serum albumin and the Docetaxel in the composition have a ratio by weight of no less than about 70:1. In some embodiments, the human serum albumin and the Docetaxel in the composition have a ratio by weight of no less than about 80:1. In some embodiments, the Docetaxel and the human serum albumin in the composition are in a ratio by weight from about 1:70 to about 1:250. In some embodiments, the Docetaxel and the human serum albumin in the composition are in a ratio by weight from about 1:80 to about 1:200. In some embodiments, the Docetaxel and the human serum albumin in the composition are in a ratio by weight from about 1:90 to about 1:200. In some embodiments, the Docetaxel and the human serum albumin in the composition are in a ratio by weight from about 1:95 to about 1:200. In some embodiments, the Docetaxel and the human serum albumin in the composition are in a ratio by weight from about 1:100 to about 1:200. In some embodiments, the Docetaxel and the human serum albumin in the composition are in a ratio by weight from about 1:80 to about 1:175. In some embodiments, the Docetaxel and the human serum albumin in the composition are in a ratio by weight from about 1:90 to about 1:175. In some embodiments, the Docetaxel and the human serum albumin in the composition are in a ratio by weight from about 1:95 to about 1:175. In some embodiments, the Docetaxel and the human serum albumin in the composition are in a ratio by weight from about 1:80 to about 1:150. In some embodiments, the Docetaxel and the human serum albumin in the composition are in a ratio by weight from about 1:90 to about 1:150. In some embodiments, the Docetaxel and the human serum albumin in the composition are in a ratio by weight from about 1:95 to about 1:150. In some embodiments, the Docetaxel and the human serum albumin in the composition are in a ratio by weight from about 1:100 to about 1:150. In some embodiments, the Docetaxel and the human serum albumin in the composition are in a ratio by weight from about 1:80 to about 1:140. In some embodiments, the Docetaxel and the human serum albumin in the composition are in a ratio by weight from about 1:85 to about 1:140. In some embodiments, the Docetaxel and the human serum albumin in the composition are in a ratio by weight from about 1:90 to about 1:140. In some embodiments, the Docetaxel and the human serum albumin in the composition are in a ratio by weight from about 1:95 to about 1:140. In some embodiments, the Docetaxel and the human serum albumin in the composition are in a ratio by weight from about 1:80 to about 1:130. In some embodiments, the Docetaxel and the human serum albumin in the composition are in a ratio by weight from about 1:90 to about 1:130. In some embodiments, the Docetaxel and the human serum albumin in the composition are in a ratio by weight from about 1:95 to about 1:130. In some embodiments, the Docetaxel and the human serum albumin in the composition are in a ratio by weight from about 1:100 to about 1:130. In some embodiments, the Docetaxel and the human serum albumin in the composition are in a ratio by weight from about 1:85 to about 1:125. In some embodiments, the Docetaxel and the human serum albumin in the composition are in a ratio by weight from about 1:90 to about 1:125. In some embodiments, the Docetaxel and the human serum albumin in the composition are in a ratio by weight from about 1:95 to about 1:125. In some embodiments, the Docetaxel and the human serum albumin in the composition are in a ratio by weight from about 1:85 to about 1:120. In some embodiments, the Docetaxel and the human serum albumin in the composition are in a ratio by weight from about 1:90 to about 1:120. In some embodiments, the Docetaxel and the human serum albumin in the composition are in a ratio by weight from about 1:90 to about 1:110. In some embodiments, the Docetaxel and the human serum albumin in the composition are in a ratio by weight from about 1:95 to about 1:105. In some embodiments, the Docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1: 140, about 1:145, about 1:150, about 1:155, about 1:160, about 1:170, about 1:180, about 1:190, about 1: 200, about 1:210, about 1:220, or about 1:250. In some embodiments, the Docetaxel and the human serum albumin in the composition are in a ratio by weight of about 1:90. In some embodiments, the Docetaxel and the human serum albumin in the composition are in a ratio by weight of about 1:95. In some embodiments, the Docetaxel and the human serum albumin in the composition are in a ratio by weight of about 1:100. In some embodiments, the Docetaxel and the human serum albumin in the composition are in a ratio by weight of about 1:105. In some embodiments, the Docetaxel and the human serum albumin in the composition are in a ratio by weight of about 1:110. In some embodiments, the Docetaxel and the human serum albumin in the composition are in a ratio by weight of about 1:115. In some embodiments, the Docetaxel and the human serum albumin in the composition are in a ratio by weight of about 1:120.

In some embodiments, the composition comprises at least one amino acid. In some embodiments, the composition comprises one amino acid. In some embodiments, the amino acid in the composition is aspartic acid. In some embodiments, the amino acid in the composition is glutamic acid. In some embodiments, the composition comprises aspartic acid. In some embodiments, the composition comprises glutamic acid. In some embodiments, the composition comprises one amino acid, which is aspartic acid. In some embodiments, the composition comprises one amino acid, which is glutamic acid. In some embodiments, the composition comprises two amino acids. In some embodiments, the composition comprises aspartic acid and glutamic acid. In some embodiments, the composition comprises two amino acids, wherein at least one amino acid in the composition is aspartic acid or glutamic acid. In some embodiments, the composition comprises two amino acids, wherein at least one amino acid in the composition is aspartic acid. In some embodiments, the composition comprises two amino acids, wherein at least one amino acid in the composition is glutamic acid. In some embodiments, the amino acid and the Docetaxel in the composition have a ratio by weight of no less than about 0.5:1. In some embodiments, the amino acid and the Docetaxel in the composition have a ratio by weight of no less than about 1:1. In some embodiments, the amino acid and the Docetaxel in the composition have a ratio by weight of no less than about 2:1. In some embodiments, the amino acid and the Docetaxel in the composition have a ratio by weight of about 2:1. In some embodiments, the amino acid and the Docetaxel in the composition have a ratio by weight of 2:1. In some embodiments, the composition comprises aspartic acid, wherein aspartic acid and the Docetaxel in the composition have a ratio by weight of no less than about 0.5:1. In some embodiments, the composition comprises aspartic acid, wherein aspartic acid and the Docetaxel in the composition have a ratio by weight of no less than about 1:1. In some embodiments, the composition comprises aspartic acid, wherein aspartic acid and the Docetaxel in the composition have a ratio by weight of about 2:1. In some embodiments, the composition comprises aspartic acid, wherein aspartic acid and the Docetaxel in the composition have a ratio by weight of 2:1. In some embodiments, the composition comprises glutamic acid, wherein glutamic acid and the Docetaxel in the composition have a ratio by weight of about 2:1. In some embodiments, the composition comprises glutamic acid, wherein glutamic acid and the Docetaxel in the composition have a ratio by weight of 2:1.

In some embodiments, the composition comprises one amino acid, wherein the amino acid and the Docetaxel in the composition have a ratio by weight from about 0.5:1 to about 50:1. In some embodiments, the composition comprises one amino acid, herein the amino acid and the Docetaxel in the composition have a ratio by weight from about 0.5:1 to about 20:1. In some embodiments, the composition comprises one amino acid, wherein the amino acid and the Docetaxel in the composition have a ratio by weight from about 0.5:1 to about 10:1. In some embodiments, the composition comprises one amino acid, wherein the amino acid and the Docetaxel in the composition have a ratio by weight from about 1:1 to about 5:1. In some embodiments, the composition comprises one amino acid, wherein the amino acid and the Docetaxel in the composition have a ratio by weight from about 1.5:1 to about 2.5:1. In some embodiments, the composition comprises one amino acid, wherein the amino acid and the Docetaxel in the composition have a ratio by weight of about 2:1. In some embodiments, the composition comprises aspartic acid, wherein aspartic acid and the Docetaxel in the composition have a ratio by weight from about 0.5:1 to about 5:1. In some embodiments, the composition comprises aspartic acid, wherein aspartic acid and the Docetaxel in the composition have a ratio by weight from about 1:1 to about 3:1. In some embodiments, the composition comprises aspartic acid, wherein aspartic acid and the Docetaxel in the composition have a ratio by weight from about 1.5:1 to about 2.5:1. In some embodiments, the composition comprises aspartic acid, wherein aspartic acid and the Docetaxel in the composition have a ratio by weight of about 2:1. In some embodiments, the composition comprises glutamic acid, wherein glutamic acid and the Docetaxel in the composition have a ratio by weight from about 0.5:1 to about 5:1. In some embodiments, the composition comprises glutamic acid, wherein glutamic acid and the Docetaxel in the composition have a ratio by weight from about 1.5:1 to about 2.5:1. In some embodiments, the composition comprises glutamic acid, wherein glutamic acid and the Docetaxel in the composition have a ratio by weight of about 2:1.

Through experiments, we surprisingly find that adding a sugar alcohol or a sugar into the composition decreases the formation of 7-Epi-docetaxel in the composition. See the experiment results described here in the example section.

In some embodiments, the composition further comprises a sugar alcohol or a sugar. In some embodiments, the composition further comprises a sugar alcohol. In some embodiments, the sugar alcohol in the composition is selected from mannitol, sorbitol, inositol, and xylitol. In some embodiments, the sugar alcohol in the composition is mannitol. In some embodiments, the sugar alcohol in the composition is sorbitol. In some embodiments, the sugar alcohol and the Docetaxel in the composition have a ratio by weight of no less than about 5:1. In some embodiments, the sugar alcohol and the Docetaxel in the composition have a ratio by weight of no less than about 10:1. In some embodiments, the sugar alcohol and the Docetaxel in the composition have a ratio by weight of no less than about 15:1. In some embodiments, the sugar alcohol and the Docetaxel in the composition have a ratio by weight of no less than about 20:1. In some embodiments, the sugar alcohol and the Docetaxel in the composition have a ratio by weight of no less than about 25:1. In some embodiments, the sugar alcohol and the Docetaxel in the composition have a ratio by weight of no less than about 50:1. In some embodiments, the sugar alcohol and the Docetaxel in the composition have a ratio by weight of no less than about 75:1. In some embodiments, the sugar alcohol and the Docetaxel in the composition have a ratio by weight of no less than about 100:1. In some embodiments, the sugar alcohol and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar alcohol and the Docetaxel in the composition have a ratio by weight of about 20:1. In some embodiments, the sugar alcohol in the composition is mannitol, in which mannitol and the Docetaxel in the composition have a ratio by weight of no less than about 5:1. In some embodiments, the sugar alcohol in the composition is mannitol, in which mannitol and the Docetaxel in the composition have a ratio by weight of no less than about 10:1. In some embodiments, the sugar alcohol in the composition is mannitol, in which mannitol and the Docetaxel in the composition have a ratio by weight of about 20:1. In some embodiments, the sugar alcohol in the composition is sorbitol, in which sorbitol and the Docetaxel in the composition have a ratio by weight of about 20:1.

In some embodiments, the sugar alcohol and the Docetaxel in the composition have a ratio by weight from about 2:1 to about 200:1. In some embodiments, the sugar alcohol and the Docetaxel in the composition have a ratio by weight from about 5:1 to about 100:1. In some embodiments, the sugar alcohol and the Docetaxel in the composition have a ratio by weight from about 5:1 to about 50:1. In some embodiments, the sugar alcohol and the Docetaxel in the composition have a ratio by weight from about 5:1 to about 40:1. In some embodiments, the sugar alcohol and the Docetaxel in the composition have a ratio by weight from about 5:1 to about 30:1. In some embodiments, the sugar alcohol and the Docetaxel in the composition have a ratio by weight from about 10:1 to about 50:1. In some embodiments, the sugar alcohol and the Docetaxel in the composition have a ratio by weight from about 10:1 to about 30:1. In some embodiments, the sugar alcohol and the Docetaxel in the composition have a ratio by weight from about 15:1 to about 30:1. In some embodiments, the sugar alcohol and the Docetaxel in the composition have a ratio by weight from about 15:1 to about 25:1. In some embodiments, the sugar alcohol and the Docetaxel in the composition have a ratio by weight from about 20:1 to about 25:1.

In some embodiments, mannitol and the Docetaxel in the composition have a ratio by weight from about 5:1 to about 50:1. In some embodiments, mannitol and the Docetaxel in the composition have a ratio by weight from about 10:1 to about 100:1. In some embodiments, mannitol and the Docetaxel in the composition have a ratio by weight from about 5:1 to about 50:1. In some embodiments, mannitol and the Docetaxel in the composition have a ratio by weight from about 5:1 to about 40:1. In some embodiments, mannitol and the Docetaxel in the composition have a ratio by weight from about 5:1 to about 30:1. In some embodiments, mannitol and the Docetaxel in the composition have a ratio by weight from about 10:1 to about 50:1. In some embodiments, mannitol and the Docetaxel in the composition have a ratio by weight from about 15:1 to about 30:1. In some embodiments, mannitol and the Docetaxel in the composition have a ratio by weight from about 15:1 to about 25:1. In some embodiments, sorbitol and the Docetaxel in the composition have a ratio by weight from about 10:1 to about 50:1. In some embodiments, sorbitol and the Docetaxel in the composition have a ratio by weight from about 15:1 to about 30:1. In some embodiments, sorbitol and the Docetaxel in the composition have a ratio by weight from about 15:1 to about 25:1.

In some embodiments, the composition further comprises a sugar. In some embodiments, the sugar in the composition is selected from glucose, sucrose, lactose, maltose, trehalose, fructose, hexose, and raffinose. In some embodiments, the sugar in the composition is glucose. In some embodiments, the sugar in the composition is lactose. In some embodiments, the sugar in the composition is sucrose. In some embodiments, the sugar in the composition is maltose. In some embodiments, the sugar and the Docetaxel in the composition have a ratio by weight of no less than about 5:1. In some embodiments, the sugar and the Docetaxel in the composition have a ratio by weight of no less than about 10:1. In some embodiments, the sugar and the Docetaxel in the composition have a ratio by weight of no less than about 15:1. In some embodiments, the sugar and the Docetaxel in the composition have a ratio by weight of no less than about 20:1. In some embodiments, the sugar and the Docetaxel in the composition have a ratio by weight of no less than about 25:1. In some embodiments, the sugar and the Docetaxel in the composition have a ratio by weight of no less than about 50:1. In some embodiments, the sugar and the Docetaxel in the composition have a ratio by weight of no less than about 75:1. In some embodiments, the sugar and the Docetaxel in the composition have a ratio by weight of no less than about 100:1. In some embodiments, the sugar and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar and the Docetaxel in the composition have a ratio by weight of about 20:1. In some embodiments, the sugar in the composition is glucose, in which glucose and the Docetaxel in the composition have a ratio by weight of no less than about 10:1. In some embodiments, the sugar in the composition is glucose, in which glucose and the Docetaxel in the composition have a ratio by weight of about 20:1. In some embodiments, the sugar in the composition is lactose, in which lactose and the Docetaxel in the composition have a ratio by weight of about 20:1.

In some embodiments, the sugar and the Docetaxel in the composition have a ratio by weight from about 2:1 to about 200:1. In some embodiments, the sugar and the Docetaxel in the composition have a ratio by weight from about 5:1 to about 100:1. In some embodiments, the sugar and the Docetaxel in the composition have a ratio by weight from about 10:1 to about 50:1. In some embodiments, the sugar and the Docetaxel in the composition have a ratio by weight from about 15:1 to about 30:1. In some embodiments, the sugar and the Docetaxel in the composition have a ratio by weight from about 15:1 to about 25:1. In some embodiments, the sugar and the Docetaxel in the composition have a ratio by weight from about 20:1 to about 25:1.

In some embodiments, glucose and the Docetaxel in the composition have a ratio by weight from about 10:1 to about 50:1. In some embodiments, glucose and the Docetaxel in the composition have a ratio by weight from about 15:1 to about 30:1. In some embodiments, glucose and the Docetaxel in the composition have a ratio by weight from about 15:1 to about 25:1. In some embodiments, lactose and the Docetaxel in the composition have a ratio by weight from about 10:1 to about 50:1. In some embodiments, lactose and the Docetaxel in the composition have a ratio by weight from about 15:1 to about 30:1. In some embodiments, lactose and the Docetaxel in the composition have a ratio by weight from about 15:1 to about 25:1.

As used herein, the term “human serum albumin” refers to native and recombinant human serum albumin. Native human serum albumin and other plasma proteins can be precipitated from human plasma by varying the pH and adding ethanol, in what is known as the Cohn fractionation process (see, e.g., Cohn E J et al., J. Am. Chem. Soc. 1946; 68:459-475). By controlling the pH and ethanol content, semi-purified fractions of plasma proteins can be produced. One of the last proteins to precipitate in the Cohn process is native human serum albumin. After precipitation, a wet paste of crude native human serum albumin is obtained. Subsequent bioprocessing steps (purification, filtration, pasteurization, etc.) can be used to produce a purified, stabilized form of native human serum albumin for commercial use (see, e.g., Lin J J et al., Pharmaceutical Research 2000; 17:391-6). Recombinant human serum albumin is a highly purified animal-, virus-, and prion-free product as alternative to native human serum albumin, to which it is structurally equivalent (see, e.g., Bosse D et al., J. Clin. Pharmacol. 2005; 45:57-67). Recombinant human serum albumin has been produced by various hosts, both prokaryotic and eukaryotic (see, e.g., Chen Z et al., Biochimica et Biophysica Acta 2013; 1830:5515-5525).

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)).

Human serum albumin (HSA) has multiple hydrophobic binding sites (a total of seven for medium and long-chain fatty acids, an endogenous ligand of HSA) and binds a diverse set of drugs, especially neutral and negatively charged hydrophobic compounds (see, e.g., Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (1981), Vorum, Dan. Med. Bull., 46, 379-99 (1999), Kragh-Hansen, Dan. Med Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (1992), and Carter et al., Adv. Protein. Chem., 45, 153-203 (1994)).

In some embodiments, the human serum albumin is a native human serum albumin. In some embodiments, the human serum albumin is a native human serum albumin obtained from pools of human plasma. In some embodiments, the human serum albumin is a recombinant human serum albumin. In some embodiments, the human serum albumin is a fatty acid free human serum albumin. In some embodiments, the human serum albumin is essentially fatty acid free.

Solutions of human serum albumin for infusion are commercially available. Those solutions must be supplemented with stabilizers to allow pasteurization and storage, to avoid the spontaneous polymerization of the albumin. Usually, N-acetyltryptophan and caprylic acid or their sodium salts are used in alone or in combination as the stabilizers.

In some embodiments, the human serum albumin is a commercially available solution of human serum albumin USP for infusion. In some embodiments, the human serum albumin comprises a commercially available solution of human serum albumin USP for infusion. In some embodiments, the human serum albumin is a lyophilized form of a commercially available solution of human serum albumin USP for infusion. In some embodiments, the human serum albumin is a lyophilized powder obtained by lyophilizing a commercially available solution of human serum albumin USP for infusion. In some embodiments, a commercially available solution of human serum albumin USP for infusion is used as the source of the human serum albumin. In some embodiments, the solution of human serum albumin USP for infusion is 5% solution of human serum albumin USP (w/v). In some embodiments, the solution of human serum albumin USP for infusion is 20% solution of human serum albumin USP (w/v). In some embodiments, the solution of human serum albumin USP for infusion is 25% solution of human serum albumin USP (w/v). In some embodiments, the human serum albumin is an aqueous solution prepared by diluting a commercially available solution of human serum albumin USP for infusion. In some embodiments, the human serum albumin is an aqueous solution prepared by diluting a commercially available solution of human serum albumin USP for infusion with water. In some embodiments, the human serum albumin is a lyophilized powder prepared from a commercially available solution of human serum albumin USP for infusion.

In some embodiments, the composition comprises at least one stabilizer for the human serum albumin. In some embodiments, the composition comprises two stabilizers for the human serum albumin. In some embodiments, the stabilizers are N-acetyltryptophan, or a pharmaceutically acceptable salt thereof, and caprylic acid, or a pharmaceutically acceptable salt thereof (e.g., sodium salt thereof). In some embodiments, the stabilizer is N-acetyltryptophan, or a pharmaceutically acceptable salt thereof (e.g., sodium salt thereof). In some embodiments, the stabilizer is caprylic acid, or a pharmaceutically acceptable salt thereof (e.g., sodium salt thereof).

In vitro studies showed that docetaxel is about 94% protein bound, mainly to α1-acid glycoprotein, albumin, and lipoproteins. In three cancer patients, the in vitro binding to plasma proteins was found to be approximately 97%. See Docetaxel Prescribing Information.

As used herein the term “docetaxel” refers to a compound that has the CAS No. 114977-28-5 and the following chemical structure:

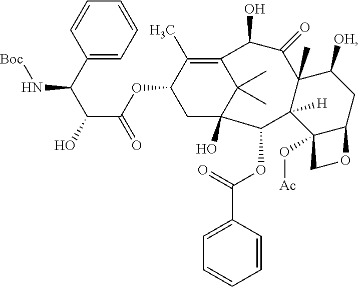

or a pharmaceutically acceptable salt thereof.

Docetaxel is a white to almost-white powder. It is highly lipophilic and practically insoluble in water.

Further, Docetaxel is a microtubule inhibitor indicated for breast cancer, non-small cell lung cancer, hormone refractory prostate cancer, gastric adenocarcinoma, and squamous cell carcinoma of the head and neck cancer.

In some embodiments, the term “docetaxel” encompasses a pharmaceutically acceptable salt of docetaxel.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. In some embodiments, pharmaceutically acceptable salts may be preferred over the respective free base or free acid because such salts impart greater stability or solubility to the molecule thereby facilitating formulation into a dosage form. Basic compounds are generally capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), napthalene-2-sulfonate, ethanedisulfonate, hydrogen bisulfide, bitartrate, gluconate, glucuronate, para-bromophenylsulfonate, carbonate, pyrosulfate, sulfite, bisulfate, monohydrogen phosphate, dihydrogen phosphate, metaphosphate, pyrophosphate, chloride, bromide, iodide, decanoate, caprylate, caprate, propiolate, suberate, sebacate, butyne-1,4-dioate, hexyne-1,6-dioate, terephthalate, sulfonate, xylenesulfonate, phenylpropionate, phenylbutyrate, β-hydroxybutyrate, glycolate, propanesulfonate, naphthalene-l-sulfonate, naphthalene-2-sulfonate and 2,5-dihydroxybenzoate. Suitable bases include pharmaceutically acceptable inorganic bases and pharmaceutically acceptable organic bases. Representative pharmaceutically acceptable base addition salts include hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the docetaxel can be a docetaxel with 1, 2, or 3 equivalents of the water solvate. In some embodiments, the docetaxel can be a docetaxel with three equivalents of the water solvate. In some embodiments, docetaxel is the docetaxel trihydrate. In some embodiments, docetaxel is the docetaxel monohydrate. In some embodiments, docetaxel is the docetaxel anhydrous. In some embodiments, the docetaxel can be a docetaxel with one equivalent of the acetone solvate. In some embodiments, the docetaxel can be any one of docetaxel solvates disclosed, for example, in WO2010091650 or US2012007167, the disclosures of which are incorporated herein by reference in its entirety.

In some embodiments, docetaxel is crystalline. In some embodiments, docetaxel is any one of the crystalline forms disclosed, for example, in WO2012115402, U.S. Pat. No. 8,410,294, US20100197944, US20100099897, U.S. Pat. No. 8,357,811, US20100160653, or US20070142457, the disclosures of which are incorporated herein by reference in their entirety.

In some embodiments, docetaxel in amorphous. In some embodiments. Docetaxel is any one of the amorphous forms disclosed, for example, in WO2008102374, the disclosure of which is incorporated herein by reference in its entirety.

As used herein the term “7-Epi-docetaxel” refers to a compound that has the CAS No. 153381-68-1 and the following chemical structure:

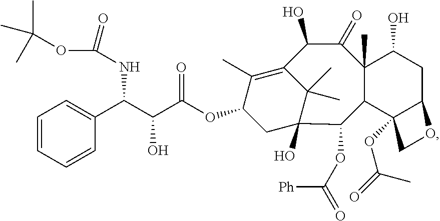 [SMILES: CC(=O)O[C@@]12CO[C@@H]1C[C@@H](O)[C@@]1(C)C(=O)[C@H](O)C3=C(C)[C@@H](OC(=O)[C@H](O)[C@@H](NC(=O)OC(C)(C)C)c4ccccc4)C[C@@](O)([C@@H](OC(=O)c4ccccc4)C12)C3(C)C]

or a pharmaceutically acceptable salt thereof.

As used herein, the term “aspartic acid” refers to a compound that has the following chemical structure:

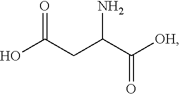

or a pharmaceutically acceptable salt thereof.

In some embodiments, aspartic acid is L-aspartic acid. The CAS Registry No. for L-aspartic acid is 56-84-8. In some embodiments, aspartic acid is D-aspartic acid. The CAS Registry No. for D-aspartic acid is 1783-96-6. In some embodiments, aspartic acid is a mixture of L-aspartic acid and D-aspartic acid. The preferred aspartic acid is L-aspartic acid.

In some embodiments, the term “aspartic acid” encompasses a pharmaceutically acceptable salt of aspartic acid. In some embodiments, the term “aspartic acid” encompasses a pharmaceutically acceptable salt of L-aspartic acid. In some embodiments, aspartic acid can be a hydrochloride salt of L-aspartic acid.

As used herein, the term “glutamic acid” refers to a compound that has the following chemical structure:

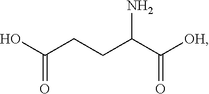

or a pharmaceutically acceptable salt thereof.

In some embodiments, glutamic acid is L-glutamic acid. The CAS Registry No. for L-glutamic acid is 56-86-0. In some embodiments, glutamic acid is D-glutamic acid. The CAS Registry No. for D-glutamic acid is 6893-26-1. In some embodiments, glutamic acid is a mixture of L-glutamic acid and D-glutamic acid. The preferred glutamic acid is L-glutamic acid.

In some embodiments, the term “glutamic acid” encompasses a pharmaceutically acceptable salt of glutamic acid. In some embodiments, the term “glutamic acid” encompasses a pharmaceutically acceptable salt of L-glutamic acid. In some embodiments, glutamic acid can be a hydrochloride salt of L-glutamic acid.

As used herein, the term “cysteine” refers to a compound that has the following chemical structure:

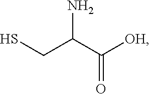

or a pharmaceutically acceptable salt thereof.

In some embodiments, cysteine is L-cysteine. The CAS Registry No. for L-cysteine is 52-90-4. In some embodiments, cysteine is D-cysteine. The CAS Registry No. for cysteine is 921-01-7. In some embodiments, cysteine is a mixture of L-cysteine and D-cysteine. The preferred cysteine is L-cysteine.

In some embodiments, the term “cysteine” encompasses a pharmaceutically acceptable salt of cysteine. In some embodiments, the term “cysteine” encompasses a pharmaceutically acceptable salt of L-cysteine. In some embodiments, cysteine can be a hydrochloride salt of L-cysteine.

As used herein, the term “sugar alcohol” refers to organic compounds, typically derived from sugars, that comprise a class of polyols. Sugar alcohols are also called polyhydric alcohols, polyalcohols, alditols or glycitols. Sugar alcohols are white, water-soluble solids that can occur naturally or be produced industrially from sugars. Suitable examples of sugar alcohol include glycerol, erythritol, threitol, arabitol, xylitol, ribitol, mannitol, sorbitol, galactiol, fucitol, inositol, malitiol, and sucrose.

As used herein, the term “sugar” refers to sweet-tasting, soluble carbohydrates. There are various types of sugar derived from different sources. Sugar includes monosaccharides, disaccharides, and oligosaccharides or polysaccharides. Fructose, galactose, and glucose are all monosaccharides. Lactose, maltose, and sucrose are all disaccharides.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use.

In some embodiments, the composition is an aqueous composition comprising t-butanol and water. In some embodiments, the said aqueous composition contains less than 40%, by weight, of t-butanol. In some embodiments, the said aqueous composition contains less than 35%, by weight, of t-butanol. In some embodiments, the said aqueous composition contains less than 30%, by weight, of t-butanol. In some embodiments, the said aqueous composition contains less than 25%, by weight, of t-butanol. In some embodiments, the said aqueous composition contains less than 20%, by weight, of t-butanol. In some embodiments, the said aqueous composition contains less than 15%, by weight, of t-butanol. In some embodiments, the said aqueous composition contains less than 10%, by weight, of t-butanol. In some embodiments, the said aqueous composition contains from about 5% to about 40%, by weight, of t-butanol. In some embodiments, the said aqueous composition contains from about 10% to about 30%, by weight, of t-butanol. In some embodiments, the said aqueous composition contains from about 15% to about 25%, by weight, of t-butanol.

In some embodiments, the composition is an aqueous composition comprising t-butanol, ethanol, and water. In some embodiments, the said aqueous composition contains less than 40%, by weight, of t-butanol and ethanol. In some embodiments, the said aqueous composition contains less than 35%, by weight, of t-butanol and ethanol. In some embodiments, the said aqueous composition contains less than 30%, by weight, of t-butanol and ethanol. In some embodiments, the said aqueous composition contains less than 25%, by weight, of t-butanol and ethanol. In some embodiments, the said aqueous composition contains less than 20%, by weight, of t-butanol and ethanol. In some embodiments, the said aqueous composition contains less than 15%, by weight, of t-butanol and ethanol. In some embodiments, the said aqueous composition contains less than 10%, by weight, of t-butanol and ethanol. In some embodiments, the said aqueous composition contains from about 5% to about 40%, by weight, of t-butanol and ethanol. In some embodiments, the said aqueous composition contains from about 10% to about 30%, by weight, of t-butanol and ethanol. In some embodiments, the said aqueous composition contains from about 15% to about 25%, by weight, of t-butanol and ethanol.

In some embodiments, the composition is a solid formulation. For example, the solid formulation can be produced in a uniform manner by lyophilization. A skilled artisan would recognize other methods, such as rotary evaporation, that can also produce solid formulations. In some embodiments, the pH of the solid formulation is neutral (e.g., pH of the composition is from about 5 to about 8, from about 5.5 to about 7.5, from about 5.5 to about 6.5, or from about 5.5 to about 7, or the pH of the composition is about 5, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the composition is an aqueous formulation. In some embodiments, the aqueous formulation is substantially free of solvent other than water. In some embodiments, the aqueous formulation is free of solvent other than water. In some embodiments, the pH of the aqueous formulation (e.g., clear aqueous solution) is neutral (e.g., pH of the composition is from about 5 to about 8, from about 5.5 to about 7.5, from about 5.5 to about 6.5, from about 5 to about 6, from about 5 to about 6.5, or from about 5.5 to about 7, or the pH of the composition is about 5, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

As used herein, “substantially free of solvent,” in reference to an aqueous solution, refers to an aqueous solution that contains less than 0.5%, by weight, of any non-water solvent. In some embodiments, the aqueous solution contains less than 0.1%, by weight, of any non-water solvent. In some embodiments, the aqueous solution contains less than 0.05%, by weight, of any non-water solvent. In some embodiments, the aqueous solution contains less than 0.01%, by weight, of any non-water solvent.

In some embodiments, the aqueous formulation can be substantially free of a surfactant selected from the group consisting of CREMOPHOR® surfactants and Polysorbate 80. In some embodiments, the aqueous formulation can be free of a surfactant selected from the group consisting of CREMOPHOR® surfactants and Polysorbate 80.

As used herein, the term “substantially free of surfactant” refers to a formulation containing less than 0.0005%, less than 0.0003%, or less than 0.0001% of a surfactant selected from the group consisting of CREMOPHOR® surfactants and Polysorbate 80.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water.

As used herein, the term “clear aqueous solution” refers to an aqueous solution containing Docetaxel and HSA that is transparent and optically clear upon visual observation and essentially free of visible particles or precipitation of undissolved Docetaxel.

The term “essentially free of visible particles or precipitation of undissolved Docetaxel” can be assessed as follows: after a clear aqueous solution is filtered with a 0.22 micron filter, the amount of Docetaxel in the filtered aqueous solution is at least 95% of the total amount of Docetaxel in the aqueous solution before filtration. The total amount of Docetaxel in the aqueous solution before filtration includes the particles or precipitation of undissolved Docetaxel in the aqueous solution or with the aqueous solution. The amount of the Docetaxel in an aqueous solution can be measured by the methods using HPLC. The methods of measuring the amount of the Docetaxel in an aqueous solution are illustrated in the experimental examples described herein. The methods are commonly understood by one of ordinary skill in the art to which this disclosure belongs.

When visually observed, for example, the term “clear aqueous solution” excludes a milky aqueous solution. Further, the term “clear aqueous solution” excludes a cloudy or hazy aqueous solution.

As used herein, the term “micron” refers to a unit of measure of one one-thousandth of a millimeter. In some embodiments, the term “micron” refers to a micrometer.

As used herein, the term “aqueous solution” refers to a solution, wherein at least one solvent is water and the weight % of water in the mixture of solvents is at least 50%, at least 60%, at least 70%, or at least 90%. In some embodiments, aqueous solution is a solution in which water is the only solvent.

As used herein, the term “aqueous solvent” refers to a liquid comprising at least 50%, at least 60%, at least 70%, at least 90% or at least 95% water. In some embodiments, aqueous solvent is water.

In some embodiments, the aqueous formulation is a clear aqueous solution reconstituted from the solid formulation (e.g. the sterile lyophilized powder) in water. In some embodiments, the aqueous formulation is a clear aqueous solution reconstituted from the solid formulation (e.g. the sterile lyophilized powder) in 0.9% saline solution. In some embodiments, the aqueous formulation is a clear aqueous solution reconstituted from the solid formulation (e.g. the sterile lyophilized powder) in 5% dextrose water solution.

In some embodiments, the aqueous formulation is a clear aqueous solution reconstituted from the solid formulation (e.g. the sterile lyophilized powder) in an aqueous solvent, wherein the aqueous formulation has pH value from about 5 to about 8. In some embodiments, the aqueous formulation is a clear aqueous solution reconstituted from the solid formulation (e.g. the sterile lyophilized powder) in water, wherein the aqueous formulation has pH value from about 5 to about 8. In some embodiments, the aqueous formulation is a clear aqueous solution reconstituted from the solid formulation (e.g. the sterile lyophilized powder) in 0.9% saline solution, wherein the aqueous formulation has pH value from about 5 to about 8. In some embodiments, the aqueous formulation is a clear aqueous solution reconstituted from the solid formulation (e.g. the sterile lyophilized powder) in 5% dextrose water solution, wherein the aqueous formulation has pH value from about 5 to about 8.

In some embodiments, the aqueous formulation is a clear aqueous solution reconstituted from the solid formulation (e.g. the sterile lyophilized powder) in an aqueous solvent, wherein the aqueous formulation has pH value from about 5.5 to about 7.5. In some embodiments, the aqueous formulation is a clear aqueous solution reconstituted from the solid formulation (e.g. the sterile lyophilized powder) in water, wherein the aqueous formulation has pH value from about 5.5 to about 7.5. In some embodiments, the aqueous formulation is a clear aqueous solution reconstituted from the solid formulation (e.g. the sterile lyophilized powder) in 0.9% saline solution, wherein the aqueous formulation has pH value from about 5.5 to about 7.5. In some embodiments, the aqueous formulation is a clear aqueous solution reconstituted from the solid formulation (e.g. the sterile lyophilized powder) in 5% dextrose water solution, wherein the aqueous formulation has pH value from about 5.5 to about 7.5.

In some aspects of the aforementioned embodiments, the concentration of the reconstituted solid (e.g., the sterile lyophilized powder comprising docetaxel, HSA, and aspartic acid or glutamic acid) in the aqueous formulation is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg , about 80 mg, about 100 mg, about 150 mg, or 200 mg per 1 ml of the aqueous solvent. In some aspects of the aforementioned embodiments, the concentration of the reconstituted solid (e.g., the sterile lyophilized powder comprising docetaxel, HSA, and aspartic acid or glutamic acid) in the aqueous formulation is from about 10 mg per 1 ml to about 250 mg per 1 ml of the aqueous solvent. In some aspects of the aforementioned embodiments, the concentration of the reconstituted solid (e.g., the sterile lyophilized powder comprising docetaxel, HSA, and aspartic acid or glutamic acid) in the aqueous formulation is from about 15 mg per 1 ml to about 100 mg per 1 ml of the aqueous solvent. In some aspects of the aforementioned embodiments, the concentration of the reconstituted solid (e.g., the sterile lyophilized powder comprising docetaxel, HSA, and aspartic acid or glutamic acid) in the aqueous formulation is from about 20 mg per 1 ml to about 50 mg per 1 ml of the aqueous solvent. In some aspects of the aforementioned embodiments, the concentration of the reconstituted solid (e.g., the sterile lyophilized powder comprising docetaxel, HSA, and aspartic acid or glutamic acid) in the aqueous formulation is from about 25 mg per 1 ml to about 40 mg per 1 ml of the aqueous solvent.

In some embodiments, the aqueous formulation has pH value from about 4 to about 9. In some embodiments, the aqueous formulation has pH value from about 5 to about 8. In some embodiments, the aqueous formulation has pH value from about 4 to about 8. In some embodiments, the aqueous formulation has pH value from about 4 to about 6.5. In some embodiments, the aqueous formulation has pH value from about 5.5 to about 7.5. In some embodiments, the aqueous formulation has pH value from about 5.5 to about 6.5. In some embodiments, the aqueous formulation has pH value from about 5 to about 6. In some embodiments, the aqueous formulation has pH value from about 5 to about 7. In some embodiments, the aqueous formulation has pH value from about 5.5 to about 7. In some embodiments, the aqueous formulation has pH value from about 5 to about 6.5. In some embodiments, the aqueous formulation has pH value from about 5.5 to about 6. In some embodiments, the aqueous formulation has pH value from about 6 to about 6.5. In some embodiments, the aqueous formulation has pH value of about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is substantially free of solvent other than water. In some embodiments, the aqueous formulation is free of solvent other than water.

In some embodiments, the aqueous formulation is a clear aqueous solution, wherein the aqueous formulation has pH value from about 5 to about 8. In some embodiments, the aqueous formulation is a clear aqueous solution, wherein the aqueous formulation has pH value from about 5 to about 8, and wherein the aqueous formulation is substantially free of solvent other than water. In some embodiments, the aqueous formulation is a clear aqueous solution, wherein the aqueous formulation has pH value from about 5 to about 8, and wherein the aqueous formulation is free of solvent other than water. In some embodiments, the aqueous formulation is a clear aqueous solution, wherein the aqueous formulation has pH value from about 5.5 to about 7, and wherein the aqueous formulation is substantially free of solvent other than water. In some embodiments, the aqueous formulation is a clear aqueous solution, wherein the aqueous formulation has pH value from about 5.5 to about 7, and wherein the aqueous formulation is free of solvent other than water.

In some embodiments, after a clear aqueous solution is filtered by a 0.22 micron filter, the amount of Docetaxel in the filtered aqueous solution is at least 95% of the total amount of Docetaxel in the aqueous solution before filtration. In some embodiments, after a clear aqueous solution is filtered by a 0.22 micron filter, the amount of Docetaxel in the filtered aqueous solution is at least 96% of the total amount of Docetaxel in the aqueous solution before filtration. In some embodiments, after a clear aqueous solution is filtered by a 0.22 micron filter, the amount of Docetaxel in the filtered aqueous solution is at least 97% of the total amount of Docetaxel in the aqueous solution before filtration. In some embodiments, after a clear aqueous solution is filtered by a 0.22 micron filter, the amount of Docetaxel in the filtered aqueous solution is at least 98% of the total amount of Docetaxel in the aqueous solution before filtration. In some embodiments, after a clear aqueous solution is filtered by a 0.22 micron filter, the amount of Docetaxel in the filtered aqueous solution is at least 99% of the total amount of Docetaxel in the aqueous solution before filtration. In some embodiments, after a clear aqueous solution is filtered by a 0.22 micron filter, the amount of Docetaxel in the filtered aqueous solution is at least 99.5% of the total amount of Docetaxel in the aqueous solution before filtration. In some embodiments, the aqueous formulation is substantially free of solvent other than water. In some embodiments, the aqueous formulation is free of solvent other than water.

In some embodiments, after the aqueous formulation (e.g. a clear aqueous solution) is filtered by a 0.22 micron filter, the amount of Docetaxel in the filtered aqueous solution is at least 95%, 96%, 97%, 98%, 99%, or 99.5% of the total amount of Docetaxel in the aqueous solution before filtration, wherein the clear aqueous solution has pH value from about 5 to about 8, and wherein the clear aqueous solution is substantially free of solvent other than water. In some embodiments, after the aqueous formulation (e.g. a clear aqueous solution) is filtered by a 0.22 micron filter, the amount of Docetaxel in the filtered aqueous solution is at least 95%, 96%, 97%, 98%, 99%, or 99.5% of the total amount of Docetaxel in the aqueous solution before filtration, wherein the clear aqueous solution has pH value from about 5.5 to about 7, and wherein the clear aqueous solution is substantially free of solvent other than water.

In some embodiments, the aqueous formulation is a clear aqueous solution for at least 1 hour, 2 hours, 3 hours, 4 hours, 5 hours, 6 hours, 7 hours, 8 hours, 10 hours, 12 hours, 24 hours, or 72 hours. In some embodiments, the aqueous formulation is a clear aqueous solution for at least 1 hour. In some embodiments, the aqueous formulation is a clear aqueous solution for at least 2 hours. In some embodiments, the aqueous formulation is a clear aqueous solution for at least 3 hours. In some embodiments, the aqueous formulation is a clear aqueous solution for at least 4 hours. In some embodiments, the aqueous formulation is a clear aqueous solution for at least 1 hour, 2 hours, 3 hours, 4 hours, 5 hours, 6 hours, 7 hours, 8 hours, 10 hours, 12 hours, or 24 hours at a temperature from about 15° C. to about 25° C. In some embodiments, the aqueous formulation is a clear aqueous solution for at least 1 hour at a temperature from about 15° C. to about 25° C. In some embodiments, the aqueous formulation is a clear aqueous solution for at least 2 hours at a temperature from about 15° C. to about 25° C. In some embodiments, the aqueous formulation is a clear aqueous solution for at least 3 hours at a temperature from about 15° C. to about 25° C. In some embodiments, the aqueous formulation is a clear aqueous solution for at least 4 hours at a temperature from about 15° C. to about 25° C. In some embodiments, the aqueous formulation is a clear aqueous solution for at least 5 hours at a temperature from about15° C. to about 25° C. In some embodiments, the aqueous formulation is a clear aqueous solution for at least 6 hours at a temperature from about 15° C. to about 25° C. In some embodiments, the aqueous formulation is a clear aqueous solution for at least 8 hours at a temperature from about 15° C. to about 25° C. In some embodiments, the aqueous formulation is a clear aqueous solution for at least 1 hour, 2 hours, 3 hours, 4 hours, 5 hours, 6 hours, 7 hours, 8 hours, 10 hours, 12 hours, 24 hours, or 72 hours at a temperature from about 1° C. to about 15° C. In some embodiments, the aqueous formulation is a clear aqueous solution for at least 3 hours at a temperature from about 1° C. to about 15° C. In some embodiments, the aqueous formulation is a clear aqueous solution for at least 6 hours at a temperature from about 1° C. to about 15° C. In some embodiments, the aqueous formulation is a clear aqueous solution for at least 12 hours at a temperature from about 1° C. to about 15° C. In some embodiments, the aqueous formulation is a clear aqueous solution for at least 24 hours at a temperature from about 1° C. to about 15° C. In some embodiments, the aqueous formulation is a clear aqueous solution for at least 1 hour, 2 hours, 3 hours, 4 hours, 5 hours, 6 hours, 7 hours, 8 hours, 10 hours, 12 hours, 24 hours, or 72 hours at a temperature from about 2° C. to about 8° C. In some embodiments, the aqueous formulation is a clear aqueous solution for at least 6 hours at a temperature from about 2° C. to about 8° C. In some embodiments, the aqueous formulation is a clear aqueous solution for at least 12 hours at a temperature from about 2° C. to about 8° C. In some embodiments, the aqueous formulation is a clear aqueous solution for at least 24 hours at a temperature from about 2° C. to about 8° C. In some embodiments, the aqueous formulation is substantially free of solvent other than water. In some embodiments, the aqueous formulation is free of solvent other than water.

In some embodiments, the aqueous formulation is a clear aqueous solution for at least 2 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 3 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 4 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 5 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 6 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 8 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 12 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is a clear aqueous solution for at least 24 hours at a temperature from about 1° C. to about 35° C., about 1° C. to about 10° C., about 10° C. to about 20° C., about 20° C. to about 25° C., or about 1° C., about 5° C., about 10° C., about 15° C., about 20° C., about 25° C., or about 30° C. In some embodiments, the aqueous formulation is substantially free of solvent other than water. In some embodiments, the aqueous formulation is free of solvent other than water.

In some embodiments, the concentration of Docetaxel in the aqueous formulation is from about 0.1 mg per 1 ml to about 1 mg per 1 ml of the aqueous solvent. In some embodiments, the concentration of Docetaxel in the aqueous formulation is from about 0.1 mg per 1 ml to about 0.8 mg per 1 ml of the aqueous solvent. In some embodiments, the concentration of Docetaxel in the aqueous formulation is from about 0.15 mg per 1 ml to about 0.5 mg per 1 ml of the aqueous solvent. In some embodiments, the concentration of Docetaxel in the aqueous formulation is from about 0.2 mg per 1 ml to about 0.4 mg per 1 ml of the aqueous solvent. In some embodiments, the concentration of Docetaxel in the aqueous formulation is from about 0.25 mg per 1 ml to about 0.35 mg per 1 ml of the aqueous solvent. In some embodiments, the concentration of Docetaxel in the aqueous formulation is from about 0.15 mg per 1 ml to about 0.3 mg per 1 ml of the aqueous solvent. In some embodiments, the concentration of Docetaxel in the aqueous formulation is from about 0.2 mg per 1 ml to about 0.3 mg per 1 ml of the aqueous solvent. In some embodiments, the concentration of Docetaxel in the aqueous formulation is from about 0.1 mg per 1 ml to about 0.25 mg per 1 ml of the aqueous solvent. In some embodiments, the concentration of Docetaxel in the aqueous formulation is from about 0.2 mg per 1 ml to about 0.29 mg per 1 ml of the aqueous solvent. In some embodiments, the concentration of Docetaxel in the aqueous formulation is about 0.15 mg per 1 ml of the aqueous solvent. In some embodiments, the concentration of Docetaxel in the aqueous formulation is about 0.2 mg per 1 ml of the aqueous solvent. In some embodiments, the concentration of Docetaxel in the aqueous formulation is about 0.25 mg per 1 ml of the aqueous solvent. In some embodiments, the concentration of Docetaxel in the aqueous formulation is about 0.33 mg per 1 ml of the aqueous solvent. In some embodiments, the concentration of Docetaxel in the aqueous formulation is about 0.4 mg per 1 ml of the aqueous solvent. In some embodiments, the concentration of Docetaxel in the aqueous formulation is about 0.5 mg per 1 ml of the aqueous solvent. In some embodiments, the aqueous formulation is substantially free of solvent other than water. In some embodiments, the aqueous formulation is free of solvent other than water.

In some embodiments, the aqueous formulation is a clear aqueous solution, wherein the concentration of Docetaxel in the aqueous formulation is from about 0.1 mg per 1 ml to about 1 mg per 1 ml of the aqueous solvent. In some embodiments, the aqueous formulation is a clear aqueous solution, wherein the concentration of Docetaxel in the aqueous formulation is from about 0.1 mg per 1 ml to about 0.5 mg per 1 ml of the aqueous solvent. In some embodiments, the aqueous formulation is a clear aqueous solution, wherein the concentration of Docetaxel in the aqueous formulation is from about 0.15 mg per 1 ml to about 0.5 mg per 1 ml of the aqueous solvent. In some embodiments, the aqueous formulation is a clear aqueous solution, wherein the concentration of Docetaxel in the aqueous formulation is from about 0.15 mg per 1 ml to about 0.33 mg per 1 ml of the aqueous solvent. In some embodiments, the aqueous formulation is a clear aqueous solution, wherein the concentration of Docetaxel in the aqueous formulation is from about 0.2 mg per 1 ml to about 0.4 mg per 1 ml of the aqueous solvent. In some embodiments, the aqueous formulation is a clear aqueous solution, wherein the concentration of Docetaxel in the aqueous formulation is from about 0.2 mg per 1 ml to about 0.3 mg per 1 ml of the aqueous solvent. In some embodiments, the aqueous formulation is a clear aqueous solution, wherein the concentration of Docetaxel in the aqueous formulation is from about 0.25 mg per 1 ml to about 0.35 mg per 1 ml of the aqueous solvent. In some embodiments, the aqueous formulation is substantially free of solvent other than water. In some embodiments, the aqueous formulation is free of solvent other than water.

In some embodiments, the aqueous formulation is a clear aqueous solution, wherein the concentration of Docetaxel in the aqueous formulation is from about 0.1 mg per 1 ml to about 1 mg per 1 ml of the aqueous solvent, and wherein the aqueous formulation is substantially free of solvent other than water. In some embodiments, the aqueous formulation is a clear aqueous solution, wherein the concentration of Docetaxel in the aqueous formulation is from about 0.15 mg per 1 ml to about 0.5 mg per 1 ml of the aqueous solvent, and wherein the aqueous formulation is substantially free of solvent other than water. In some embodiments, the aqueous formulation is a clear aqueous solution, wherein the concentration of Docetaxel in the aqueous formulation is from about 0.2 mg per 1 ml to about 0.4 mg per 1 ml of the aqueous solvent, and wherein the aqueous formulation is substantially free of solvent other than water. In some embodiments, the aqueous formulation is a clear aqueous solution, wherein the concentration of Docetaxel in the aqueous formulation is from about 0.2 mg per 1 ml to about 0.3 mg per 1 ml of the aqueous solvent, and wherein the aqueous formulation is substantially free of solvent other than water.

In some embodiments, the aqueous formulation is a clear aqueous solution, wherein the concentration of Docetaxel in the aqueous formulation is from about 0.1 mg per 1 ml to about 1 mg per 1 ml of the aqueous solvent, and wherein after the clear aqueous solution is filtered by a 0.22 micron filter, the amount of Docetaxel in the filtered aqueous solution is at least 95% of the total amount of Docetaxel in the aqueous solution before filtration. In some embodiments, the aqueous formulation is a clear aqueous solution, wherein the concentration of Docetaxel in the aqueous formulation is from about 0.1 mg per 1 ml to about 1 mg per 1 ml of the aqueous solvent, and wherein after the clear aqueous solution is filtered by a 0.22 micron filter, the amount of Docetaxel in the filtered aqueous solution is at least 96% of the total amount of Docetaxel in the aqueous solution before filtration. In some embodiments, the aqueous formulation is a clear aqueous solution, wherein the concentration of Docetaxel in the aqueous formulation is from about 0.1 mg per 1 ml to about 1 mg per 1 ml of the aqueous solvent, and wherein after the clear aqueous solution is filtered by a 0.22 micron filter, the amount of Docetaxel in the filtered aqueous solution is at least 98% of the total amount of Docetaxel in the aqueous solution before filtration.

Also, provided herein is a pharmaceutical composition comprising the composition comprising Docetaxel, human serum albumin, and one or more amino acids selected from aspartic acid, glutamic acid, and cysteine, as described herein, and a pharmaceutically acceptable carrier. In some embodiments, provided herein is a pharmaceutical composition comprising Docetaxel, human serum albumin, one or more amino acids selected from aspartic acid, glutamic acid, and cysteine, and a sugar alcohol or a sugar as described herein, and a pharmaceutically acceptable carrier.

In some embodiments, the pharmaceutical composition further comprises at least one anti-cancer drug (e.g., any one of the anti-cancer drugs as described herein). In some embodiments, the pharmaceutical composition further comprises a stabilizer selected from sodium caprylate and sodium N-acetyltryptophanate.

As used herein, the term “pharmaceutically acceptable carrier” is meant any solution used to solubilize and deliver an agent to a subject. A desirable pharmaceutically acceptable carrier is saline or water. Other pharmaceutically acceptable carrier and their formulation are known to one skilled in the art and described, for example, in Remington's Pharmaceutical Sciences. (20^{th} edition), ed. A. Gennaro, 2003, Lippincon Williams & Wilkins.

Pharmaceutically acceptable carriers that may be used in the pharmaceutical compositions of the present application include, but are not limited to, ion exchangers, alumina, aluminum stearate, lecithin, serum proteins (other than HSA), buffer substances such as phosphates, glycine, sorbic acid, potassium sorbate, salts or electrolytes, such as protamine sulfate, disodium hydrogen phosphate, potassium hydrogen phosphate, sodium chloride, zinc salts, colloidal silica, magnesium trisilicate, and cellulose-based substances.

In some embodiments, the pharmaceutical composition is free of a surfactant selected from the group consisting of CREMOPHOR® surfactants and Polysorbate 80. In some embodiments, the pharmaceutical composition is substantially free of a surfactant selected from the group consisting of CREMOPHOR® surfactants and Polysorbate 80.

Also, provided herein is a method of treating cancer comprising the step of administering to a subject in need thereof a therapeutically effective amount of a pharmaceutical composition comprising the composition comprising Docetaxel, human serum albumin, and one or more amino acids selected from aspartic acid, glutamic acid, and cysteine as described herein, and a pharmaceutically acceptable carrier. In some embodiments, provided herein is a method of treating cancer comprising the step of administering to a subject in need thereof a therapeutically effective amount of a pharmaceutical composition comprising the composition comprising Docetaxel, human serum albumin, one or more amino acids selected from aspartic acid, glutamic acid, and cysteine, and a sugar alcohol or a sugar as described herein, and a pharmaceutically acceptable carrier.

As used herein, the terms “individual”, “patient”, or “subject” are used interchangeably and refer to any animal, including mammals, preferably mice, rats, other rodents, rabbits, dogs, cats, swine, cattle, sheep, horses, or primates, and most preferably humans.

In some embodiments, the cancer is selected from the group consisting of bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

In some embodiments, cancer is selected from sarcoma, angiosarcoma, fibrosarcoma, rhabdomyosarcoma, liposarcoma, myxoma, rhabdomyoma, fibroma, lipoma, teratoma, non-small cell lung cancer (NSCLC), bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer , cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

As used herein, an “effective amount,” “therapeutically effective amount,” or a “pharmaceutically-effective amount” in reference to the compounds or compositions of the instant invention refers to the amount sufficient to induce a desired biological, pharmacological, or therapeutic outcome in a subject. That result can be reduction, mitigation, delay, shortening the time to resolution of, alleviation of the signs or symptoms of, or exert a medically-beneficial effect upon the underlying pathophysiology or pathogenesis of an expected or observed side-effect, toxicity, disorder or condition, or any other desired alteration of a biological system. In cancer treatment, the result will generally include the reduction, mitigation, limitation, and/or, delay of the deleterious physiological manifestations, growth or metastases of neoplasms.

In some embodiments, the cancer is a solid tumor cancer. In some embodiments, the cancer is selected from the group consisting of breast cancer, non-small cell lung cancer, prostate cancer, gastric cancer, head and neck cancer, ovarian cancer, pancreatic cancer, and Kaposi's sarcoma. In some embodiments, the cancer is a breast cancer. In some embodiments, the cancer is a non-small cell lung cancer. In some embodiments, the cancer is a prostate cancer. In some embodiments, the cancer is a gastric cancer. In some embodiments, the cancer is a head and neck cancer. In some embodiments the cancer is an ovarian cancer. In some embodiments, the cancer is a pancreatic cancer. In some embodiments, the cancer is a Kaposi's sarcoma.

In some embodiments, the method of treating cancer (e.g. any one of cancers described herein) comprises the step of administering to a subject in need thereof of a therapeutically effective amount of a composition comprising Docetaxel, proteins comprising a human serum albumin, at least one amino acid selected from aspartic acid, glutamic acid, and cysteine, and optionally a sugar alcohol or a sugar, as described herein, and a therapeutically effective amount of at least one inhibitor of the following kinases for the treatment of cancer: PIM, Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf.

In some embodiments, the method of treating cancer (e.g. any one of cancers described herein) comprises the step of administering to a subject in need thereof of a therapeutically effective amount of a pharmaceutical composition comprising the composition comprising Docetaxel, proteins comprising a human serum albumin, at least one amino acid selected from aspartic acid, glutamic acid, and cysteine, and optionally a sugar alcohol or a sugar, as described herein, and a therapeutically effective amount of at least one anti-cancer drug. Examples of an anti-cancer drug include aberaterone, aberaterone acetate, abarelix, aldesleukin, alemtuzumab, alitretinoin, allopurinol, altretamine, anastrozole, arsenic trioxide, asparaginase, azacitidine, bavituximab, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, enzalutamide, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

In some embodiments, a composition comprising Docetaxel, human serum albumin, and one or more amino acids selected from aspartic acid, glutamic acid, and cysteine, as described herein and an anti-cancer drug are administered simultaneously (e.g., in the same dosage form or in separate dosage forms).

In some embodiments, a composition comprising Docetaxel, human serum albumin, and one or more amino acids selected from aspartic acid, glutamic acid, and cysteine, as described herein and an anti-cancer drug are administered consecutively (e.g., anti-cancer drug is administered to the subject before or after the docetaxel composition).

The composition comprising Docetaxel, human serum albumin, and one or more amino acids selected from aspartic acid, glutamic acid, and cysteine, as described herein, can be administered to an individual, such as human, via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. For example, the composition can be administered by inhalation to treat conditions of the respiratory tract. The composition can be used to treat respiratory conditions such as pulmonary fibrosis, broncheolitis obliterans, lung cancer, bronchoalveolar carcinoma, and the like. In some embodiments, the nanoparticle composition is administrated intravenously. In some embodiments, the composition comprising Docetaxel, at least one amino acid selected from aspartic acid, glutamic acid, and cysteine, and optionally a sugar alcohol or a sugar, as described herein, can be administered to an individual, such as human, via intravenous route.

The methods described herein may be performed alone or in conjunction with another therapy, such as surgery, radiation, chemotherapy, immunotherapy, gene therapy, and the like. Additionally, a person having a greater risk of developing the proliferative disease may receive treatments to inhibit or and/or delay the development of the disease.

As will be understood by those of ordinary skill in the art, the appropriate doses of Docetaxel will be approximately those already employed in clinical therapies wherein Docetaxel is administered alone or in combination with other chemotherapeutic agents. Variation in dosage will likely occur depending on the condition being treated. Appropriate effective doses will also vary, as recognized by those skilled in the art, depending on the severity of the disease, the route of administration, the sex, age and general health condition of the subject, excipient usage, the possibility of co-usage with other therapeutic treatments such as use of other agents, and the judgment of the treating physician. For example, guidance for selecting an effective dose can be determined by reference to the prescribing information for Docetaxel.

Also, provided herein is a composition comprising Docetaxel, human serum albumin, and a sugar alcohol or a sugar, wherein the human serum albumin and the Docetaxel in the composition have a ratio by weight of no less than 60:1. In some embodiments, provided herein is a composition comprising Docetaxel, human serum albumin, and a sugar alcohol, wherein the human serum albumin and the Docetaxel in the composition have a ratio by weight of no less than 60:1. In some embodiments, provided herein is a composition comprising Docetaxel, human serum albumin, and a sugar, wherein the human serum albumin and the Docetaxel in the composition have a ratio by weight of no less than 60:1. In some embodiments, the pH of a composition is neutral (e.g., pH of the composition is from about 5 to about 8, from about 5.5 to about 7.5, or from about 5.5 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the human serum albumin and the Docetaxel in the composition have a ratio by weight of no less than about 70:1. In some embodiments, the Docetaxel and the human serum albumin in the composition are in a ratio by weight from about 1:70 to about 1:250. In some embodiments, the Docetaxel and the human serum albumin in the composition are in a ratio by weight from about 1:80 to about 1:200. In some embodiments, the Docetaxel and the human serum albumin in the composition are in a ratio by weight from about 1:90 to about 1:200. In some embodiments, the Docetaxel and the human serum albumin in the composition are in a ratio by weight from about 1:95 to about 1:200. In some embodiments, the Docetaxel and the human serum albumin in the composition are in a ratio by weight from about 1:100 to about 1:200. In some embodiments, the Docetaxel and the human serum albumin in the composition are in a ratio by weight from about 1:80 to about 1:175. In some embodiments, the Docetaxel and the human serum albumin in the composition are in a ratio by weight from about 1:90 to about 1:175. In some embodiments, the Docetaxel and the human serum albumin in the composition are in a ratio by weight from about 1:95 to about 1:175. In some embodiments, the Docetaxel and the human serum albumin in the composition are in a ratio by weight from about 1:80 to about 1:150. In some embodiments, the Docetaxel and the human serum albumin in the composition are in a ratio by weight from about 1:90 to about 1:150. In some embodiments, the Docetaxel and the human serum albumin in the composition are in a ratio by weight from about 1:95 to about 1:150. In some embodiments, the Docetaxel and the human serum albumin in the composition are in a ratio by weight from about 1:100 to about 1:150. In some embodiments, the Docetaxel and the human serum albumin in the composition are in a ratio by weight from about 1:80 to about 1:140. In some embodiments, the Docetaxel and the human serum albumin in the composition are in a ratio by weight from about 1:85 to about 1:140. In some embodiments, the Docetaxel and the human serum albumin in the composition are in a ratio by weight from about 1:90 to about 1:140. In some embodiments, the Docetaxel and the human serum albumin in the composition are in a ratio by weight from about 1:95 to about 1:140. In some embodiments, the Docetaxel and the human serum albumin in the composition are in a ratio by weight from about 1:80 to about 1:130. In some embodiments, the Docetaxel and the human serum albumin in the composition are in a ratio by weight from about 1:90 to about 1:130. In some embodiments, the Docetaxel and the human serum albumin in the composition are in a ratio by weight from about 1:95 to about 1:130. In some embodiments, the Docetaxel and the human serum albumin in the composition are in a ratio by weight from about 1:100 to about 1:130. In some embodiments, the Docetaxel and the human serum albumin in the composition are in a ratio by weight from about 1:85 to about 1:125. In some embodiments, the Docetaxel and the human serum albumin in the composition are in a ratio by weight from about 1:90 to about 1:125. In some embodiments, the Docetaxel and the human serum albumin in the composition are in a ratio by weight from about 1:95 to about 1:125. In some embodiments, the Docetaxel and the human serum albumin in the composition are in a ratio by weight from about 1:85 to about 1:120. In some embodiments, the Docetaxel and the human serum albumin in the composition are in a ratio by weight from about 1:90 to about 1:120. In some embodiments, the Docetaxel and the human serum albumin in the composition are in a ratio by weight from about 1:90 to about 1:110. In some embodiments, the Docetaxel and the human serum albumin in the composition are in a ratio by weight from about 1:95 to about 1:105. In some embodiments, the Docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1: 140, about 1:145, about 1:150, about 1:155, about 1:160, about 1:170, about 1:180, about 1:190, about 1:200, about 1:210, about 1:220, or about 1: 250. In some embodiments, the Docetaxel and the human serum albumin in the composition are in a ratio by weight of about 1:90. In some embodiments, the Docetaxel and the human serum albumin in the composition are in a ratio by weight of about 1:95. In some embodiments, the Docetaxel and the human serum albumin in the composition are in a ratio by weight of about 1:100. In some embodiments, the Docetaxel and the human serum albumin in the composition are in a ratio by weight of about 1:105. In some embodiments, the Docetaxel and the human serum albumin in the composition are in a ratio by weight of about 1:110. In some embodiments, the Docetaxel and the human serum albumin in the composition are in a ratio by weight of about 1:115. In some embodiments, the Docetaxel and the human serum albumin in the composition are in a ratio by weight of about 1:120.

In some embodiments, the composition comprises a sugar alcohol. In some embodiments, the sugar alcohol in the composition is selected from mannitol, sorbitol, inositol, and xylitol. In some embodiments, the sugar alcohol in the composition is mannitol. In some embodiments, the sugar alcohol in the composition is sorbitol. In some embodiments, the sugar alcohol and the Docetaxel in the composition have a ratio by weight of no less than about 5:1. In some embodiments, the sugar alcohol and the Docetaxel in the composition have a ratio by weight of no less than about 10:1. In some embodiments, the sugar alcohol and the Docetaxel in the composition have a ratio by weight of no less than about 15:1. In some embodiments, the sugar alcohol and the Docetaxel in the composition have a ratio by weight of no less than about 20:1. In some embodiments, the sugar alcohol and the Docetaxel in the composition have a ratio by weight of no less than about 25:1. In some embodiments, the sugar alcohol and the Docetaxel in the composition have a ratio by weight of no less than about 50:1. In some embodiments, the sugar alcohol and the Docetaxel in the composition have a ratio by weight of no less than about 75:1. In some embodiments, the sugar alcohol and the Docetaxel in the composition have a ratio by weight of no less than about 100:1. In some embodiments, the sugar alcohol and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar alcohol and the Docetaxel in the composition have a ratio by weight of about 20:1. In some embodiments, mannitol and the Docetaxel in the composition have a ratio by weight of about 20:1. In some embodiments, sorbitol and the Docetaxel in the composition have a ratio by weight of about 20:1.

In some embodiments, the sugar alcohol and the Docetaxel in the composition have a ratio by weight from about 2:1 to about 200:1. In some embodiments, the sugar alcohol and the Docetaxel in the composition have a ratio by weight from about 5:1 to about 100:1. In some embodiments, the sugar alcohol and the Docetaxel in the composition have a ratio by weight from about 10:1 to about 50:1. In some embodiments, the sugar alcohol and the Docetaxel in the composition have a ratio by weight from about 15:1 to about 30:1. In some embodiments, the sugar alcohol and the Docetaxel in the composition have a ratio by weight from about 15:1 to about 25:1. In some embodiments, the sugar alcohol and the Docetaxel in the composition have a ratio by weight from about 20:1 to about 25:1.

In some embodiments, mannitol and the Docetaxel in the composition have a ratio by weight from about 10:1 to about 50:1. In some embodiments, mannitol and the Docetaxel in the composition have a ratio by weight from about 15:1 to about 30:1. In some embodiments, mannitol and the Docetaxel in the composition have a ratio by weight from about 15:1 to about 25:1. In some embodiments, sorbitol and the Docetaxel in the composition have a ratio by weight from about 10:1 to about 50:1. In some embodiments, sorbitol and the Docetaxel in the composition have a ratio by weight from about 15:1 to about 30:1. In some embodiments, sorbitol and the Docetaxel in the composition have a ratio by weight from about 15:1 to about 25:1.

In some embodiments, the composition comprises a sugar. In some embodiments, the sugar in the composition is selected from glucose, sucrose, lactose, maltose, trehalose, fructose, hexose, and raffinose. In some embodiments, the sugar in the composition is glucose. In some embodiments, the sugar in the composition is lactose. In some embodiments, the sugar in the composition is sucrose. In some embodiments, the sugar in the composition is maltose. In some embodiments, the sugar and the Docetaxel in the composition have a ratio by weight of no less than about 5:1. In some embodiments, the sugar and the Docetaxel in the composition have a ratio by weight of no less than about 10:1. In some embodiments, the sugar and the Docetaxel in the composition have a ratio by weight of no less than about 15:1. In some embodiments, the sugar and the Docetaxel in the composition have a ratio by weight of no less than about 20:1. In some embodiments, the sugar and the Docetaxel in the composition have a ratio by weight of no less than about 25:1. In some embodiments, the sugar and the Docetaxel in the composition have a ratio by weight of no less than about 50:1. In some embodiments, the sugar and the Docetaxel in the composition have a ratio by weight of no less than about 75:1. In some embodiments, the sugar and the Docetaxel in the composition have a ratio by weight of no less than about 100:1. In some embodiments, the sugar and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar and the Docetaxel in the composition have a ratio by weight of about 20:1. In some embodiments, glucose and the Docetaxel in the composition have a ratio by weight of about 20:1. In some embodiments, lactose and the Docetaxel in the composition have a ratio by weight of about 20:1.

In some embodiments, the sugar and the Docetaxel in the composition have a ratio by weight from about 2:1 to about 200:1. In some embodiments, the sugar and the Docetaxel in the composition have a ratio by weight from about 5:1 to about 100:1. In some embodiments, the sugar and the Docetaxel in the composition have a ratio by weight from about 10:1 to about 50:1. In some embodiments, the sugar and the Docetaxel in the composition have a ratio by weight from about 15:1 to about 30:1. In some embodiments, the sugar and the Docetaxel in the composition have a ratio by weight from about 15:1 to about 25:1. In some embodiments, the sugar and the Docetaxel in the composition have a ratio by weight from about 20:1 to about 25:1.

In some embodiments, glucose and the Docetaxel in the composition have a ratio by weight from about 10:1 to about 50:1. In some embodiments, glucose and the Docetaxel in the composition have a ratio by weight from about 15:1 to about 30:1. In some embodiments, lucose and the Docetaxel in the composition have a ratio by weight from about 15:1 to about 25:1. In some embodiments, lactose and the Docetaxel in the composition have a ratio by weight from about 10:1 to about 50:1. In some embodiments, lactose and the Docetaxel in the composition have a ratio by weight from about 15:1 to about 30:1. In some embodiments, lactose and the Docetaxel in the composition have a ratio by weight from about 15:1 to about 25:1.

In some embodiments, 7-Epi-docetaxel and the Docetaxel in the composition have a ratio by weight of no more than about 1:100. In some embodiments, 7-Epi-docetaxel and the Docetaxel in the composition have a ratio by weight of no more than about 1:200. In some embodiments, 7-Epi-docetaxel and the Docetaxel in the composition have a ratio by weight of no more than about 1:300. In some embodiments, 7-Epi-docetaxel and the Docetaxel in the composition have a ratio by weight of no more than about 1:500. In some embodiments, 7-Epi-docetaxel and the Docetaxel in the composition have a ratio by weight of no more than about 1:1000. In some embodiments, 7-Epi-docetaxel and the Docetaxel in the composition have a ratio by weight of no more than about 1:2000.

In some embodiments, the human serum albumin is a native human serum albumin. In some embodiments, the human serum albumin is a native human serum albumin obtained from pools of human plasma. In some embodiments, the human serum albumin is a recombinant human serum albumin. In some embodiments, the human serum albumin is a fatty acid free human serum albumin. In some embodiments, the human serum albumin is essentially fatty acid free.

Also, provided herein is a composition comprising Docetaxel and human serum albumin, wherein 7-Epi-docetaxel and the Docetaxel in the composition have a ratio by weight of no more than 1:100, and wherein the human serum albumin and the Docetaxel in the composition have a ratio by weight of no less than 60:1. In some embodiments, the pH of a composition is neutral (e.g., pH of the composition is from about 5 to about 8, from about 5.5 to about 7.5, or from about 5.5 to about 7, or the pH of the composition is about 5, about 5.5, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, 7-Epi-docetaxel and the Docetaxel in the composition have a ratio by weight of no more than 1:200. In some embodiments, 7-Epi-docetaxel and the Docetaxel in the composition have a ratio by weight of no more than 1:300. In some embodiments, 7-Epi-docetaxel and the Docetaxel in the composition have a ratio by weight of no more than 1:500. In some embodiments, 7-Epi-docetaxel and the Docetaxel in the composition have a ratio by weight of no more than 1:1000. In some embodiments, 7-Epi-docetaxel and the Docetaxel in the composition have a ratio by weight of no more than 1:2000. In some embodiments, 7-Epi-docetaxel and the Docetaxel in the composition have a ratio by weight of no more than about 1:200. In some embodiments, 7-Epi-docetaxel and the Docetaxel in the composition have a ratio by weight of no more than about 1:300. In some embodiments, 7-Epi-docetaxel and the Docetaxel in the composition have a ratio by weight of no more than about 1:500. In some embodiments, 7-Epi-docetaxel and the Docetaxel in the composition have a ratio by weight of no more than about 1:1000. In some embodiments,

In some embodiments, the human serum albumin and the Docetaxel in the composition have a ratio by weight of no less than 70:1. In some embodiments, the Docetaxel and the human serum albumin in the composition are in a ratio by weight from about 1:70 to about 1:250. In some embodiments, the Docetaxel and the human serum albumin in the composition are in a ratio by weight from about 1:80 to about 1:200. In some embodiments, the Docetaxel and the human serum albumin in the composition are in a ratio by weight from about 1:90 to about 1:200. In some embodiments, the Docetaxel and the human serum albumin in the composition are in a ratio by weight from about 1:95 to about 1:200. In some embodiments, the Docetaxel and the human serum albumin in the composition are in a ratio by weight from about 1:100 to about 1:200. In some embodiments, the Docetaxel and the human serum albumin in the composition are in a ratio by weight from about 1:80 to about 1:175. In some embodiments, the Docetaxel and the human serum albumin in the composition are in a ratio by weight from about 1:90 to about 1:175. In some embodiments, the Docetaxel and the human serum albumin in the composition are in a ratio by weight from about 1:95 to about 1:175. In some embodiments, the Docetaxel and the human serum albumin in the composition are in a ratio by weight from about 1:80 to about 1:150. In some embodiments, the Docetaxel and the human serum albumin in the composition are in a ratio by weight from about 1:90 to about 1:150. In some embodiments, the Docetaxel and the human serum albumin in the composition are in a ratio by weight from about 1:95 to about 1:150. In some embodiments, the Docetaxel and the human serum albumin in the composition are in a ratio by weight from about 1:100 to about 1:150. In some embodiments, the Docetaxel and the human serum albumin in the composition are in a ratio by weight from about 1:80 to about 1:140. In some embodiments, the Docetaxel and the human serum albumin in the composition are in a ratio by weight from about 1:85 to about 1:140. In some embodiments, the Docetaxel and the human serum albumin in the composition are in a ratio by weight from about 1:90 to about 1:140. In some embodiments, the Docetaxel and the human serum albumin in the composition are in a ratio by weight from about 1:95 to about 1:140. In some embodiments, the Docetaxel and the human serum albumin in the composition are in a ratio by weight from about 1:80 to about 1:130. In some embodiments, the Docetaxel and the human serum albumin in the composition are in a ratio by weight from about 1:90 to about 1:130. In some embodiments, the Docetaxel and the human serum albumin in the composition are in a ratio by weight from about 1:95 to about 1:130. In some embodiments, the Docetaxel and the human serum albumin in the composition are in a ratio by weight from about 1:100 to about 1:130. In some embodiments, the Docetaxel and the human serum albumin in the composition are in a ratio by weight from about 1:85 to about 1:125. In some embodiments, the Docetaxel and the human serum albumin in the composition are in a ratio by weight from about 1:90 to about 1:125. In some embodiments, the Docetaxel and the human serum albumin in the composition are in a ratio by weight from about 1:95 to about 1:125. In some embodiments, the Docetaxel and the human serum albumin in the composition are in a ratio by weight from about 1:85 to about 1:120. In some embodiments, the Docetaxel and the human serum albumin in the composition are in a ratio by weight from about 1:90 to about 1:120. In some embodiments, the Docetaxel and the human serum albumin in the composition are in a ratio by weight from about 1:90 to about 1:110. In some embodiments, the Docetaxel and the human serum albumin in the composition are in a ratio by weight from about 1:95 to about 1:105. In some embodiments, the Docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1:140, about 1:145, about 1:150, about 1:155, about 1:160, about 1:170, about 1:180, about 1:190, about 1: 200, about 1:210, about 1:220, or about 1:250. In some embodiments, the Docetaxel and the human serum albumin in the composition are in a ratio by weight of about 1:90. In some embodiments, the Docetaxel and the human serum albumin in the composition are in a ratio by weight of about 1:95. In some embodiments, the Docetaxel and the human serum albumin in the composition are in a ratio by weight of about 1:100. In some embodiments, the Docetaxel and the human serum albumin in the composition are in a ratio by weight of about 1:105. In some embodiments, the Docetaxel and the human serum albumin in the composition are in a ratio by weight of about 1:110. In some embodiments, the Docetaxel and the human serum albumin in the composition are in a ratio by weight of about 1:115. In some embodiments, the Docetaxel and the human serum albumin in the composition are in a ratio by weight of about 1:120.

In some embodiments, the human serum albumin is a native human serum albumin. In some embodiments, the human serum albumin is a native human serum albumin obtained from pools of human plasma. In some embodiments, the human serum albumin is a recombinant human serum albumin. In some embodiments, the human serum albumin is a fatty acid free human serum albumin. In some embodiments, the human serum albumin is essentially fatty acid free.

In some embodiments, the composition further comprises a sugar alcohol or a sugar. In some embodiments, the composition further comprises a sugar alcohol. In some embodiments, the sugar alcohol in the composition is selected from mannitol, sorbitol, inositol, and xylitol. In some embodiments, the sugar alcohol in the composition is mannitol. In some embodiments, the sugar alcohol in the composition is sorbitol. In some embodiments, the sugar alcohol and the Docetaxel in the composition have a ratio by weight of no less than about 5:1. In some embodiments, the sugar alcohol and the Docetaxel in the composition have a ratio by weight of no less than about 10:1. In some embodiments, the sugar alcohol and the Docetaxel in the composition have a ratio by weight of no less than about 15:1. In some embodiments, the sugar alcohol and the Docetaxel in the composition have a ratio by weight of no less than about 20:1. In some embodiments, the sugar alcohol and the Docetaxel in the composition have a ratio by weight of no less than about 25:1. In some embodiments, the sugar alcohol and the Docetaxel in the composition have a ratio by weight of no less than about 50:1. In some embodiments, the sugar alcohol and the Docetaxel in the composition have a ratio by weight of no less than about 75:1. In some embodiments, the sugar alcohol and the Docetaxel in the composition have a ratio by weight of no less than about 100:1. In some embodiments, the sugar alcohol and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar alcohol and the Docetaxel in the composition have a ratio by weight of about 20:1. In some embodiments, mannitol and the Docetaxel in the composition have a ratio by weight of about 20:1.

In some embodiments, the composition further comprises a sugar. In some embodiments, the sugar in the composition is selected from glucose, sucrose, lactose, maltose, trehalose, fructose, hexose, and raffinose. In some embodiments, the sugar in the composition is glucose. In some embodiments, the sugar in the composition is lactose. In some embodiments, the sugar and the Docetaxel in the composition have a ratio by weight of no less than about 5:1. In some embodiments, the sugar and the Docetaxel in the composition have a ratio by weight of no less than about 10:1. In some embodiments, the sugar and the Docetaxel in the composition have a ratio by weight of no less than about 15:1. In some embodiments, the sugar and the Docetaxel in the composition have a ratio by weight of no less than about 20:1. In some embodiments, the sugar and the Docetaxel in the composition have a ratio by weight of no less than about 25:1. In some embodiments, the sugar and the Docetaxel in the composition have a ratio by weight of no less than about 50:1. In some embodiments, the sugar and the Docetaxel in the composition have a ratio by weight of no less than about 75:1. In some embodiments, the sugar and the Docetaxel in the composition have a ratio by weight of no less than about 100:1. In some embodiments, the sugar and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar and the Docetaxel in the composition have a ratio by weight of about 20:1. In some embodiments, glucose and the Docetaxel in the composition have a ratio by weight of about 20:1.

In some embodiments, the composition is an aqueous formulation. In some embodiments, the aqueous formulation is substantially free of solvent other than water. In some embodiments, the aqueous formulation is free of solvent other than water. In some embodiments, the pH of the aqueous formulation (e.g., clear aqueous solution) is neutral (e.g., pH of the composition is from about 5 to about 8, from about 5.5 to about 7.5, from about 5.5 to about 6.5, or from about 5.5 to about 7, or the pH of the composition is about 5, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5 or about 8).

In some embodiments, the aqueous formulation is a clear aqueous solution. In some embodiments, the aqueous formulation is a clear aqueous solution, wherein the aqueous formulation is substantially free of solvent other than water.

In some embodiments, the aqueous formulation has pH value from about 4 to about 9. In some embodiments, the aqueous formulation has pH value from about 5 to about 8. In some embodiments, the aqueous formulation has pH value from about 5.5 to about 7.5. In some embodiments, the aqueous formulation has pH value from about 5.5 to about 6.5. In some embodiments, the aqueous formulation has pH value from about 5 to about 7. In some embodiments, the aqueous formulation has pH value from about 5.5 to about 7. In some embodiments, the aqueous formulation has pH value from about 5 to about 6.5. In some embodiments, the aqueous formulation has pH value from about 5 to about 6. In some embodiments, the aqueous formulation has pH value from about 5.5 to about 6. In some embodiments, the aqueous formulation has pH value from about 6 to about 6.5. In some embodiments, the aqueous formulation has pH value of about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is substantially free of solvent other than water. In some embodiments, the aqueous formulation is free of solvent other than water.

In some embodiments, the aqueous formulation is a clear aqueous solution for at least 1 hour, 2 hours, 3 hours, 4 hours, 5 hours, 6 hours, 7 hours, 8 hours, 10 hours, 12 hours, 24 hours, or 72 hours. In some embodiments, the aqueous formulation is a clear aqueous solution for at least 1 hour, 2 hours, 3 hours, 4 hours, 5 hours, 6 hours, 7 hours, 8 hours, 10 hours, 12 hours, or 24 hours at a temperature from about 15° C. to about 25° C. In some embodiments, the aqueous formulation is a clear aqueous solution for at least 1 hour, 2 hours, 3 hours, 4 hours, 5 hours, 6 hours, 7 hours, 8 hours, 10 hours, 12 hours, or 24 hours at a temperature from about 2° C. to about 8° C. In some embodiments, the aqueous formulation is substantially free of solvent other than water.

Also, provided herein is a liquid composition comprising Docetaxel and human serum albumin, wherein the human serum albumin and the Docetaxel in the composition have a ratio by weight of no less than 60:1, wherein the composition comprises water, t-butanol, and ethanol, wherein the pH of the composition is from about 4 to about 8, and wherein t-butanol and ethanol in the composition are in a ratio from about 1:20 to about 100:1 (v/v). In some embodiments, the amount of water and the combined amount of t-butanol and ethanol in the composition are in a ratio from about 3:2 to about 10:1 (v/v). In some embodiments, the liquid composition is a clear aqueous solution.

In some embodiments, the liquid composition comprises an amino acid. In some embodiments, the liquid composition comprises aspartic acid. In some embodiments, the liquid composition comprises glutamic acid. In some embodiments, the liquid composition comprises a sugar alcohol or a sugar. In some embodiments, the liquid composition comprises a sugar alcohol. In some embodiments, the liquid composition comprises a sugar. In some embodiments, the liquid composition comprises mannitol.

In some embodiments, the human serum albumin and the Docetaxel in the composition have a ratio by weight of no less than about 70:1. In some embodiments, the human serum albumin and the Docetaxel in the composition have a ratio by weight of no less than about 80:1. In some embodiments, the Docetaxel and the human serum albumin in the composition are in a ratio by weight from about 1:70 to about 1:250. In some embodiments, the Docetaxel and the human serum albumin in the composition are in a ratio by weight from about 1:80 to about 1:200. In some embodiments, the Docetaxel and the human serum albumin in the composition are in a ratio by weight from about 1:90 to about 1:200. In some embodiments, the Docetaxel and the human serum albumin in the composition are in a ratio by weight from about 1:95 to about 1:200. In some embodiments, the Docetaxel and the human serum albumin in the composition are in a ratio by weight from about 1:100 to about 1:200. In some embodiments, the Docetaxel and the human serum albumin in the composition are in a ratio by weight from about 1:80 to about 1:175. In some embodiments, the Docetaxel and the human serum albumin in the composition are in a ratio by weight from about 1:90 to about 1:175. In some embodiments, the Docetaxel and the human serum albumin in the composition are in a ratio by weight from about 1:95 to about 1:175. In some embodiments, the Docetaxel and the human serum albumin in the composition are in a ratio by weight from about 1:80 to about 1:150. In some embodiments, the Docetaxel and the human serum albumin in the composition are in a ratio by weight from about 1:90 to about 1:150. In some embodiments, the Docetaxel and the human serum albumin in the composition are in a ratio by weight from about 1:95 to about 1:150. In some embodiments, the Docetaxel and the human serum albumin in the composition are in a ratio by weight from about 1:100 to about 1:150. In some embodiments, the Docetaxel and the human serum albumin in the composition are in a ratio by weight from about 1:80 to about 1:140. In some embodiments, the Docetaxel and the human serum albumin in the composition are in a ratio by weight from about 1:85 to about 1:140. In some embodiments, the Docetaxel and the human serum albumin in the composition are in a ratio by weight from about 1:90 to about 1:140. In some embodiments, the Docetaxel and the human serum albumin in the composition are in a ratio by weight from about 1:95 to about 1:140. In some embodiments, the Docetaxel and the human serum albumin in the composition are in a ratio by weight from about 1:80 to about 1:130. In some embodiments, the Docetaxel and the human serum albumin in the composition are in a ratio by weight from about 1:90 to about 1:130. In some embodiments, the Docetaxel and the human serum albumin in the composition are in a ratio by weight from about 1:95 to about 1:130. In some embodiments, the Docetaxel and the human serum albumin in the composition are in a ratio by weight from about 1:100 to about 1:130. In some embodiments, the Docetaxel and the human serum albumin in the composition are in a ratio by weight from about 1:85 to about 1:125. In some embodiments, the Docetaxel and the human serum albumin in the composition are in a ratio by weight from about 1:90 to about 1:125. In some embodiments, the Docetaxel and the human serum albumin in the composition are in a ratio by weight from about 1:95 to about 1:125. In some embodiments, the Docetaxel and the human serum albumin in the composition are in a ratio by weight from about 1:85 to about 1:120. In some embodiments, the Docetaxel and the human serum albumin in the composition are in a ratio by weight from about 1:90 to about 1:120. In some embodiments, the Docetaxel and the human serum albumin in the composition are in a ratio by weight from about 1:90 to about 1:110. In some embodiments, the Docetaxel and the human serum albumin in the composition are in a ratio by weight from about 1:95 to about 1:105. In some embodiments, the Docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1: 140, about 1:145, about 1:150, about 1:155, about 1:160, about 1:170, about 1:180, about 1:190, about 1: 200, about 1:210, about 1:220, or about 1:250. In some embodiments, the Docetaxel and the human serum albumin in the composition are in a ratio by weight of about 1:90. In some embodiments, the Docetaxel and the human serum albumin in the composition are in a ratio by weight of about 1:95. In some embodiments, the Docetaxel and the human serum albumin in the composition are in a ratio by weight of about 1:100. In some embodiments, the Docetaxel and the human serum albumin in the composition are in a ratio by weight of about 1:105. In some embodiments, the Docetaxel and the human serum albumin in the composition are in a ratio by weight of about 1:110. In some embodiments, the Docetaxel and the human serum albumin in the composition are in a ratio by weight of about 1:115. In some embodiments, the Docetaxel and the human serum albumin in the composition are in a ratio by weight of about 1:120.

In some embodiments, the composition has pH value from about 5 to about 8. In some embodiments, the composition has pH value from about 4 to about 7. In some embodiments, the composition has pH value from about 5 to about 7. In some embodiments, the composition has pH value from about 4 to about 6.5. In some embodiments, the composition has pH value from about 5.5 to about 7.5. In some embodiments, the composition has pH value from about 5 to about 6.5. In some embodiments, the composition has pH value from about 4.5 to about 7.5. In some embodiments, the composition has pH value from about 5.5 to about 7. In some embodiments, the composition has pH value from about 4.5 to about 6.5. In some embodiments, the composition has pH value from about 5.5 to about 6.5. In some embodiments, the composition has pH value from about 5.0 to about 6.0. In some embodiments, the composition has pH value from about 5.7 to about 6.3. In some embodiments, the composition has pH value of about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

In some embodiments, t-butanol and ethanol in the composition are in a ratio from about 1:20 to about 75:1 (v/v). In some embodiments, t-butanol and ethanol in the composition are in a ratio from about 1:15 to about 75:1 (v/v). In some embodiments, t-butanol and ethanol in the composition are in a ratio from about 1:10 to about 75:1 (v/v). In some embodiments, t-butanol and ethanol in the composition are in a ratio from about 1:5 to about 75:1 (v/v). In some embodiments, t-butanol and ethanol in the composition are in a ratio from about 1:3 to about 75:1 (v/v). In some embodiments, t-butanol and ethanol in the composition are in a ratio from about 1:20 to about 50:1 (v/v). In some embodiments, t-butanol and ethanol in the composition are in a ratio from about 1:15 to about 50:1 (v/v). In some embodiments, t-butanol and ethanol in the composition are in a ratio from about 1:10 to about 50:1 (v/v). In some embodiments, t-butanol and ethanol in the composition are in a ratio from about 1:5 to about 50:1 (v/v). In some embodiments, t-butanol and ethanol in the composition are in a ratio from about 1:10 to about 40:1 (v/v). In some embodiments, t-butanol and ethanol in the composition are in a ratio from about 1:5 to about 40:1 (v/v). In some embodiments, t-butanol and ethanol in the composition are in a ratio from about 1:5 to about 30:1 (v/v). In some embodiments, t-butanol and ethanol in the composition are in a ratio from about 1:5 to about 20:1 (v/v). In some embodiments, t-butanol and ethanol in the composition are in a ratio from about 1:3 to about 40:1 (v/v). In some embodiments, t-butanol and ethanol in the composition are in a ratio from about 1:3 to about 30:1 (v/v). In some embodiments, t-butanol and ethanol in the composition are in a ratio from about 1:3 to about 20:1 (v/v). In some embodiments, t-butanol and ethanol in the composition are in a ratio from about 1:2 to about 40:1 (v/v). In some embodiments, t-butanol and ethanol in the composition are in a ratio from about 1:2 to about 30:1 (v/v). In some embodiments, t-butanol and ethanol in the composition are in a ratio from about 1:2 to about 20:1 (v/v). In some embodiments, t-butanol and ethanol in the composition are in a ratio from about 1:1 to about 40:1 (v/v). In some embodiments, t-butanol and ethanol in the composition are in a ratio from about 1:1 to about 30:1 (v/v). In some embodiments, t-butanol and ethanol in the composition are in a ratio from about 1:1 to about 20:1 (v/v). In some embodiments, t-butanol and ethanol in the composition are in a ratio from about 1:1 to about 15:1 (v/v). In some embodiments, t-butanol and ethanol in the composition are in a ratio from about 1:1 to about 10:1 (v/v). In some embodiments, t-butanol and ethanol in the composition are in a ratio of about 1:1, about 2:1, about 3:1, about 4:1, about 5:1, about 6:1, about 7:1, about 8:1, about 9:1, about 15:1, or about 19:1. In some embodiments, t-butanol and ethanol in the composition are in a ratio of about 7:1.

In some embodiments, the amount of water and the combined amount of t-butanol and ethanol in the composition are in a ratio from about 3:2 to about 5:1 (v/v). In some embodiments, the amount of water and the combined amount of t-butanol and ethanol in the composition are in a ratio from about 3:2 to about 4:1 (v/v). In some embodiments, the amount of water and the combined amount of t-butanol and ethanol in the composition are in a ratio from about 3:2 to about 10:3 (v/v). In some embodiments, the amount of water and the combined amount of t-butanol and ethanol in the composition are in a ratio from about 3:2 to about 3:1 (v/v). In some embodiments, the amount of water and the combined amount of t-butanol and ethanol in the composition are in a ratio from about 2:1 to about 10:1 (v/v). In some embodiments, the amount of water and the combined amount of t-butanol and ethanol in the composition are in a ratio from about 2:1 to about 5:1 (v/v). In some embodiments, the amount of water and the combined amount of t-butanol and ethanol in the composition are in a ratio from about 2:1 to about 4:1 (v/v). In some embodiments, the amount of water and the combined amount of t-butanol and ethanol in the composition are in a ratio from about 2:1 to about 10:3 (v/v). In some embodiments, the amount of water and the combined amount of t-butanol and ethanol in the composition are in a ratio from about 2:1 to about 3:1 (v/v). In some embodiments, the amount of water and the combined amount of t-butanol and ethanol in the composition are in a ratio of about 5:2 (v/v).

In some embodiments, the concentration of Docetaxel in the composition is from about 0.1 mg per 1 ml to about 2 mg per 1 ml. In some embodiments, the concentration of Docetaxel in the composition is from about 0.1 mg per 1 ml to about 1.5 mg per 1 ml. In some embodiments, the concentration of Docetaxel in the composition is from about 0.2 mg per 1 ml to about 2 mg per 1 ml. In some embodiments, the concentration of Docetaxel in the composition is from about 0.2 mg per 1 ml to about 1.5 mg per 1 ml. 1 ml to about 2 mg per 1 ml. In some embodiments, the concentration of Docetaxel in the composition is from about 0.2 mg per 1 ml to about 1.0 mg per 1 ml. In some embodiments, the concentration of Docetaxel in the composition is from about 0.25 mg per 1 ml to about 1.5 mg per 1 ml. In some embodiments, the concentration of Docetaxel in the composition is from about 0.5 mg per 1 ml to about 1.5 mg per 1 ml. In some embodiments, the concentration of Docetaxel in the composition is from about 0.25 mg per 1 ml to about 1 mg per 1 ml. In some embodiments, the concentration of Docetaxel in the composition is from about 0.5 mg per 1 ml to about 1 mg per 1 ml. In some embodiments, the concentration of Docetaxel in the composition is from about 0.3 mg per 1 ml to about 0.9 mg per 1 ml. In some embodiments, the concentration of Docetaxel in the composition is from about 0.4 mg per 1 ml to about 0.9 mg per 1 ml. In some embodiments, the concentration of Docetaxel in the composition is from about 0.5 mg per 1 ml to about 0.9 mg per 1 ml. In some embodiments, the concentration of Docetaxel in the composition is from about 0.5 mg per 1 ml to about 0.8 mg per 1 ml. In some embodiments, the concentration of Docetaxel in the composition is from about 0.6 mg per 1 ml to about 0.8 mg per 1 ml.

In some embodiments, after the composition is filtered by a 0.22 micron filter, the amount of Docetaxel in the filtered aqueous solution is at least 95% of the total amount of Docetaxel in the liquid composition before filtration. In some embodiments, after the composition is filtered by a 0.22 micron filter, the amount of Docetaxel in the filtered aqueous solution is at least 96% of the total amount of Docetaxel in the liquid composition before filtration. In some embodiments, after the composition is filtered by a 0.22 micron filter, the amount of Docetaxel in the filtered aqueous solution is at least 97% of the total amount of Docetaxel in the liquid composition before filtration. In some embodiments, after the composition is filtered by a 0.22 micron filter, the amount of Docetaxel in the filtered aqueous solution is at least 98% of the total amount of Docetaxel in the liquid composition before filtration. In some embodiments, after the composition is filtered by a 0.22 micron filter, the amount of Docetaxel in the filtered aqueous solution is at least 99% of the total amount of Docetaxel in the liquid composition before filtration.

In some embodiments, the liquid composition is a clear aqueous solution for at least 1 hour, 2 hours, 3 hours, 4 hours, 5 hours, 6 hours, or 8 hours. In some embodiments, the liquid composition is a clear aqueous solution for at least 1 hour. In some embodiments, the liquid composition is a clear aqueous solution for at least 2 hours. In some embodiments, the liquid composition is a clear aqueous solution for at least 3 hours. In some embodiments, the liquid composition is a clear aqueous solution for at least 4 hours. In some embodiments, the liquid composition is a clear aqueous solution for at least 6 hours.

Also, provided herein is a solid composition comprising Docetaxel, human serum albumin, and aspartic acid, wherein human serum albumin and the Docetaxel in the composition have a ratio by weight from about 90:1 to about 150:1, wherein aspartic acid and the Docetaxel in the composition have a ratio by weight from about 1:1 to about 3:1, and wherein 7-Epi-docetaxel and the Docetaxel in the composition have a ratio by weight of no more than 1:125. In some embodiments, the composition further comprises mannitol, wherein mannitol and the Docetaxel in the composition have a ratio by weight of no less than 5:1.

In some embodiments, the Docetaxel and the human serum albumin in the composition are in a ratio by weight from about 1:90 to about 1:120. In some embodiments, the Docetaxel and the human serum albumin in the composition are in a ratio by weight of about 1:100. In some embodiments, aspartic acid and the Docetaxel in the composition have a ratio by weight from about 1:1 to about 3:1. In some embodiments, the Docetaxel and the human serum albumin in the composition are in a ratio by weight of about 1:100. In some embodiments, aspartic acid and the Docetaxel in the composition have a ratio by weight of about 2:1. In some embodiments, mannitol and the Docetaxel in the composition have a ratio by weight from about 10:1 to about 50:1. In some embodiments, mannitol and the Docetaxel in the composition have a ratio by weight of about 20:1. In some embodiments, 7-Epi-docetaxel and the Docetaxel in the composition have a ratio by weight of no more than 1:200. In some embodiments, 7-Epi-docetaxel and the Docetaxel in the composition have a ratio by weight of no more than 1:300. In some embodiments, 7-Epi-docetaxel and the Docetaxel in the composition have a ratio by weight of no more than 1:500.

In some embodiments, the solid composition comprises 10 mg of Docetaxel. In some embodiments, the solid composition comprises about 10 mg of Docetaxel. In some embodiments, the solid composition comprises about 10 mg of Docetaxel, about 20 mg of aspartic acid, and about 200 mg of mannitol. In some embodiments, the solid composition comprises about 10 mg of Docetaxel, about 1 g of human serum albumin, about 20 mg of aspartic acid, and about 200 mg of mannitol. In some embodiments, the solid composition comprises about 10 mg of Docetaxel, about 1 g of human serum albumin, and about 20 mg of aspartic acid. In some embodiments, the solid composition comprises about 10 mg of Docetaxel, and about 1 g of human serum albumin. In some embodiments, the solid composition is produced by lyophilization.

In some embodiments, the solid composition contains t-butanol and ethanol as residual solvents. In some embodiments, the amount of residual t-butanol in the composition is no more than 3%. In some embodiments, the amount of residual t-butanol in the composition is no more than 2.5%. In some embodiments, the amount of residual t-butanol in the composition is no more than 2%. In some embodiments, the amount of residual t-butanol in the composition is no more than 1%. In some embodiments, the amount of residual t-butanol in the composition is no more than 0.5%. In some embodiments, the amount of residual ethanol in the composition is no more than 1%. In some embodiments, the amount of residual ethanol in the composition is no more than 0.5%. In some embodiments, the amount of residual ethanol in the composition is no more than 0.2%. In some embodiments, the amount of residual ethanol in the composition is no more than 0.1%.

### Kits

The present invention also includes pharmaceutical kits useful, for example, in the treatment or prevention of any one of diseases or disorders referred to herein, which include one or more containers containing a pharmaceutical composition comprising a composition of docetaxel and the human serum albumin as described herein. Such kits can further include, if desired, one or more of various conventional pharmaceutical kit components, such as, for example, containers with one or more pharmaceutically acceptable carriers (e.g., water, 0.9% saline, or 5% dextrose), additional containers, etc., as will be readily apparent to those skilled in the art. Instructions, either as inserts or as labels, indicating quantities of the components to be administered (e.g., dosage amounts as described herein), guidelines for administration, and/or guidelines for mixing the components, can also be included in the kit.

### Methods of Making

Also, provided herein are methods to prepare a composition comprising Docetaxel, human serum albumin, and one or more amino acids selected from aspartic acid, glutamic acid, and cysteine, as described herein, or a composition comprising Docetaxel, human serum albumin, one or more amino acids selected from aspartic acid, glutamic acid, and cysteine, and a sugar alcohol or a sugar, as described herein.

In some embodiments, the method comprises mixing an organic solution of Docetaxel in a polar water-miscible organic solvent and a first aqueous solution containing human serum albumin to form a second aqueous solution, wherein the second aqueous solution is a clear solution.

In some embodiments, the method further comprises removing said polar water-miscible organic solvent and water from the second aqueous solution.

In some embodiments, the method comprises adding an organic solution of Docetaxel in a polar water-miscible organic solvent containing ethanol into a first aqueous solution comprising human serum albumin and t-butanol to form a second aqueous solution, wherein the second aqueous solution is a clear solution.

In some embodiments, the method comprises adding an organic solution of Docetaxel in a polar water-miscible organic solvent containing ethanol into a first aqueous solution comprising human serum albumin and t-butanol to form a second aqueous solution.

In some embodiments, the method comprises adding an organic solution of Docetaxel in ethanol or in a mixture of ethanol and t-butanol into a first aqueous solution comprising human serum albumin, aspartic acid, and t-butanol to form a second aqueous solution, wherein the second aqueous solution is a clear solution.

In some embodiments, the method comprises adding an organic solution of Docetaxel in ethanol or in a mixture of ethanol and t-butanol into a first aqueous solution comprising human serum albumin, aspartic acid, and t-butanol to form a second aqueous solution.

In some embodiments, the method comprises adding an organic solution of Docetaxel in ethanol into a first aqueous solution comprising human serum albumin, aspartic acid, and t-butanol to form a second aqueous solution.

In some embodiments, the method comprises adding an organic solution of Docetaxel in ethanol or in a mixture of ethanol and t-butanol into a first aqueous solution comprising human serum albumin, glutamic acid, and t-butanol to form a second aqueous solution, wherein the second aqueous solution is a clear solution.

A non-limiting preferred method is as follows.

### Formation of the Organic Solution

In some embodiments, Docetaxel is dissolved in a polar organic solvent (e.g., an alcohol such as methanol, ethanol, t-butanol, and/or isopropanol; acetone, THF, CH₃CN; DMF; or mixtures thereof) to form an organic solution.

As used herein, the term “organic solution” refers to a solution wherein at least one solvent is a non-aqueous solvent and the weight % of the non-aqueous solvent in the mixture of solvents is at least 50%, at least 60%, at least 70% or at least 90%. In some embodiments, organic solution is a solution in which does not comprise water as a solvent.

In some embodiments, the terms “organic solvent” and “non-aqueous solvent” are used interchangeably and refer to a liquid comprising is at least 50%, at least 60%, at least 70%, at least 90%, or at least 95% of a solvent other than water.

The polar organic solvent is miscible in water. In some embodiments, the polar organic solvent is an alcohol. In some embodiments, the polar organic solvent is ethanol. In some embodiments, the polar organic solvent comprises ethanol. In some embodiments, the polar organic solvent comprises an alcohol. In some embodiments, the polar organic solvent is ethanol or t-butanol, or mixtures thereof. In some embodiments, the polar organic solvent can be acetone. In some embodiments, the polar organic solvent is a mixture of ethanol and t-butanol. In some embodiments, the polar organic solvent is a mixture of ethanol and t-butanol, in which the ratio of (v/v) ethanol and t-butanol is from about 20:1 to about 1:100. In some embodiments, the polar organic solvent is a mixture of ethanol and t-butanol, in which the ratio of (v/v) ethanol and t-butanol is from about 5:1 to about 1:50. In some embodiments, the polar organic solvent is a mixture of ethanol and t-butanol, in which the ratio of (v/v) ethanol and t-butanol is from about 1:1 to about 1:20. In some embodiments, the polar organic solvent is a mixture of ethanol and t-butanol, in which the ratio of (v/v) ethanol and t-butanol is from about 1:2 to about 1:10. In some embodiments, the polar organic solvent is a mixture of ethanol and t-butanol, in which the ratio of (v/v) ethanol and t-butanol is from about 1:4 to about 1:10. In some embodiments, the polar organic solvent is a mixture of ethanol and t-butanol, in which the ratio of (v/v) ethanol and t-butanol is from about 1:5 to about 1:8. In some embodiments, the polar organic solvent is a mixture of ethanol and t-butanol, in which the ratio of (v/v) ethanol and t-butanol is about 1:8. In some embodiments, the polar organic solvent is a mixture of ethanol and t-butanol, in which the ratio of (v/v) ethanol and t-butanol is about 1:7. In some embodiments, the polar organic solvent is a mixture of ethanol and t-butanol, in which the ratio of (v/v) ethanol and t-butanol is about 1:6. In some embodiments, the polar organic solvent is a mixture of ethanol and t-butanol, in which the ratio of (v/v) ethanol and t-butanol is about 1:5. In some embodiments, the polar organic solvent is a mixture of ethanol and t-butanol, in which the ratio of (v/v) ethanol and t-butanol is about 1:4.

### Formation of the First Aqueous Solution

In some embodiments, the first aqueous solution comprises an intravenous Human Albumin (human serum albumin) solution for infusion (e.g. Human Albumin USP; prepared as a 5%, 20%, or 25% protein solution). In some embodiments, the first aqueous solution comprises an intravenous Human Albumin (human serum albumin) solution for infusion (e.g. Human Albumin USP; prepared as a 5%, 20%, or 25% protein solution), and one or more amino acids selected from aspartic acid, glutamic acid, or cysteine. In some embodiments, the first aqueous solution comprises an intravenous Human Albumin (human serum albumin) solution for infusion (e.g. Human Albumin USP; prepared as a 5%, 20%, or 25% protein solution), one or more amino acids selected from aspartic acid, glutamic acid, and cysteine, and a sugar alcohol or a sugar. In some embodiments, the first aqueous solution comprises an intravenous Human Albumin (human serum albumin) solution for infusion (e.g. Human Albumin USP; prepared as a 5%, 20%, or 25% protein solution), one or more amino acids selected from aspartic acid, glutamic acid, and cysteine, and a sugar alcohol or a sugar in water. In some embodiments, the first aqueous solution comprises an intravenous Human Albumin (human serum albumin) solution for infusion (e.g. Human Albumin USP; prepared as a 5%, 20%, or 25% protein solution), aspartic acid, and a sugar alcohol or a sugar in water. In some embodiments, the first aqueous solution comprises an intravenous Human Albumin (human serum albumin) solution for infusion (e.g. Human Albumin USP; prepared as a 5%, 20%, or 25% protein solution), glutamic acid, and a sugar alcohol or a sugar in water. In some embodiments, the first aqueous solution comprises an intravenous Human Albumin (human serum albumin) solution for infusion (e.g. Human Albumin USP; prepared as a 5%, 20%, or 25% protein solution), aspartic acid, and mannitol.

In some embodiments, the first aqueous solution comprises polar organic solvents. In some embodiments, the first aqueous solution comprises an alcohol. In some embodiments, the first aqueous solution comprises ethanol or t-butanol, or mixtures thereof. In some embodiments, the first aqueous solution comprises t-butanol. In some embodiments, the first aqueous solution comprises ethanol. In some embodiments, the first aqueous solution comprises aspartic acid. In some embodiments, the first aqueous solution comprises glutamic acid. In some embodiments, the first aqueous solution comprises a sugar alcohol or a sugar. In some embodiments, the first aqueous solution comprises mannitol. In some embodiments, the first aqueous solution comprises human serum albumin, aspartic acid, mannitol, and t-butanol.

In some embodiments, the amount of aqueous solvent in the first aqueous solution is from about 0.2 mL to about 10 mL per 100 mg of human serum albumin. In some embodiments, the amount of aqueous solvent in the first aqueous solution is from about 0.4 mL to about 5 mL per 100 mg of human serum albumin. In some embodiments, the amount of aqueous solvent in the first aqueous solution is from about 0.5 mL to about 2.5 mL per 100 mg of human serum albumin. In some embodiments, the amount of aqueous solvent in the first aqueous solution is from about 0.75 mL to about 2 mL per 100 mg of human serum albumin. In some embodiments, the amount of aqueous solvent in the first aqueous solution is from about 0.8 mL to about 1.5 mL per 100 mg of human serum albumin. In some embodiments, the amount of aqueous solvent in the first aqueous solution is from about 0.9 mL to about 1.2 mL per 100 mg of human serum albumin. In some embodiments, the amount of aqueous solvent in the first aqueous solution is about 0.25 mL, about 0.5 mL, about 0.75 mL, about 0.9 mL, about 1 mL, about 1.1 mL, about 1.2 mL, about 1.5 mL, or about 2 mL per 100 mg of human serum albumin.

In some embodiments, the resulting composition comprising Docetaxel, human serum albumin, one or more amino acids selected from aspartic acid, glutamic acid, or cysteine, and a sugar alcohol or a sugar can have any molar ratio or any ratio by weight of the Docetaxel to the human serum albumin as described herein.

In some embodiments, the preparation of the organic solution and the preparation of the first aqueous solution are performed concurrently.

In some embodiments, the preparation of the organic solution and the preparation of the first aqueous solution are performed sequentially. In some embodiments, the preparation of the organic solution is performed before the preparation of the first aqueous solution. In some embodiments, the preparation of the first aqueous solution is performed before the preparation of the organic solution.

In some embodiments, the range of pH in the first aqueous solution is from about 3 to about 9, from about 4 to about 8, from about 5 to about 8, from about 5 to about 7, from about 6 to about 7, from about 3 to about 5, from about 3 to about 7, from about 4 to about 6, or from about 6 to about 6.5. In some embodiments, the pH of the first aqueous solution is about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, or about 8.

### Formation of the Second Aqueous Solution

In some embodiments, the organic solution of Docetaxel is mixed with the first aqueous solution to form a second aqueous solution. In some embodiments, the second aqueous solution is a clear aqueous solution.

In some embodiments, the volume ratio of the amount of water to the amount of the polar organic solvent in the second aqueous solution is in a range from about 1:1 to about 1000:1. In some embodiments, the volume ratio of the amount of water to the amount of the polar organic solvent in the second aqueous solution is in a range from about 1.5:1 to about 100:1. In some embodiments, the volume ratio of the amount of water to the amount of the polar organic solvent in the second aqueous solution is in a range from about 1.5:1 to about 10:1. In some embodiments, the volume ratio of the amount of water to the amount of the polar organic solvent in the second aqueous solution is in a range from about 1.5:1 to about 5:1. In some embodiments, the volume ratio of the amount of water to the amount of the polar organic solvent in the second aqueous solution is in a range from about 2:1 to about 3:1. In some embodiments, the volume ratio of the amount of water to the amount of the polar organic solvent in the second aqueous solution is about 1.5:1, about 2:1, about 2.2:1, about 2.3:1, about 2.4:1, about 2.5:1, about 3:1, about 4:1, about 5:1, about 6:1, about 7:1, about 8:1, about 9:1, or about 10:1.

In some embodiments, the organic solution is added to the first aqueous solution to form a second aqueous solution. In some embodiments, the organic solution is added dropwise to the first aqueous solution to form a second aqueous solution. In some embodiments, the mixing is performed with agitation. In some embodiments, the mixing is performed with stirring.

In some embodiments, the time of addition is in a range from about 0.05 min to about 2 hour. In some embodiments, the time of addition is in a range from about 0.05 min to about 0.5 hour. In some embodiments, the time of addition is in a range from about 0.1 min to about 15 min. In some embodiments, the time of addition is less than about 0.5 hour. .In some embodiments, the time of addition is less than 0.5 hour. In some embodiments, the time of addition is less than about 15 min. In some embodiments, the time of addition is less than about 10 min. In some embodiments, the time of addition is less than about 5 min.

In some embodiments, the addition is done at the temperature from about 0° C. to about 35° C. In some embodiments, the addition is done at the temperature from about 0° C. to about 25° C. In some embodiments, the addition is done at the temperature from about 0° C. to about 10° C. In some embodiments, the addition is done at the temperature from about 0° C. to about 5° C. In some embodiments, the addition is done at the temperature from about 5° C. to about 10° C.

In some embodiments, the range of pH in the second aqueous solution is from about 3 to about 9, from about 4 to about 8, from about 5 to about 8, from about 5 to about 7, from about 6 to about 7, from about 3 to about 5, from about 3 to about 7, from about 4 to about 6, or from about 6 to about 6.5. In some embodiments, the pH of the first aqueous solution is about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, or about 8.

In some embodiments, the second aqueous solution comprises docetaxel, human serum albumin, aspartic acid, mannitol, ethanol, and t-butanol. In some embodiments, the second aqueous solution comprises docetaxel, human serum albumin, aspartic acid, a sugar alcohol or a sugar, ethanol, and t-butanol. In some embodiments, the second aqueous solution comprises t-butanol. In some embodiments, the second aqueous solution comprises ethanol. In some embodiments, the second aqueous solution comprises t-butanol and ethanol. In some embodiments, the second aqueous solution comprises t-butanol. Ethanol, and water as solvents.

Removal of Solvents from the Second Aqueous Solution

In some embodiments, the solvents including both water and organic solvent are removed from the second aqueous solution to provide a solid composition. In some embodiments, the solvents including both water and organic solvent are removed from the second aqueous solution simultaneously to provide a solid composition. In some embodiments, the solvents are removed by lyophilization.

In some embodiments, the second aqueous solution is filtered before removal of solvents. For example, the second aqueous solution can be filtered by a 0.22 micron filter before removal of solvents.

As used herein, the term “micron” refers to a unit of measure of one one-thousandth of a millimeter.

### Reconstitution of the Solid Composition

In some embodiments, the solid composition is mixed with water. In some embodiments, the solid composition is mixed with an aqueous solution of 0.45% saline and 2.5% Dextrose. In some embodiments, the aqueous solution is a 0.9% saline solution. In some embodiments, the aqueous solution is a 5% Dextrose solution. In some embodiments, the mixing is the addition of water or the water solution to the solid. In some embodiments, the mixing is the addition of the solid to water or the water solution. In some embodiments, the mixing reconstitutes the solid. In some embodiments, the mixing yields a clear aqueous solution. In some embodiments, the range of pH in the reconstituted solution is from about 5 to about 8, from about 5.5 to about 7.5, from about 5 to about 7, from about 5.5 to about 6.5, from about 4 to about 6, or from about 5 to about 6.5.

### Composition Prepared by the Process

In some embodiments, the present disclosure provides a composition comprising

Docetaxel, human serum albumin, and one or more amino acids selected from aspartic acid, glutamic acid, or cysteine, and a sugar alcohol or a sugar as described herein, produced by a method comprising the steps of:

(i) obtaining an organic solution of docetaxel in a polar water-miscible organic solvent;

(ii) obtaining a first aqueous solution comprising human serum albumin, one or more amino acids selected from aspartic acid, glutamic acid, and cysteine, and a sugar alcohol or a sugar; and

(iii) adding the organic solution of docetaxel into the first aqueous solution to obtain a second aqueous solution comprising the composition comprising Docetaxel, human serum albumin, and one or more amino acids selected from aspartic acid, glutamic acid, and cysteine, and a sugar alcohol or a sugar.

In some embodiments, the human serum albumin and the Docetaxel in the composition have a ratio by weight of no less than about 70:1. In some embodiments, the Docetaxel and the human serum albumin in the composition are in a ratio by weight from about 1:70 to about 1:250. In some embodiments, the Docetaxel and the human serum albumin in the composition are in a ratio by weight from about 1:80 to about 1:200. In some embodiments, the Docetaxel and the human serum albumin in the composition are in a ratio by weight from about 1:90 to about 1:200. In some embodiments, the Docetaxel and the human serum albumin in the composition are in a ratio by weight from about 1:95 to about 1:200. In some embodiments, the Docetaxel and the human serum albumin in the composition are in a ratio by weight from about 1:100 to about 1:200. In some embodiments, the Docetaxel and the human serum albumin in the composition are in a ratio by weight from about 1:80 to about 1:175. In some embodiments, the Docetaxel and the human serum albumin in the composition are in a ratio by weight from about 1:90 to about 1:175. In some embodiments, the Docetaxel and the human serum albumin in the composition are in a ratio by weight from about 1:95 to about 1:175. In some embodiments, the Docetaxel and the human serum albumin in the composition are in a ratio by weight from about 1:80 to about 1:150. In some embodiments, the Docetaxel and the human serum albumin in the composition are in a ratio by weight from about 1:90 to about 1:150. In some embodiments, the Docetaxel and the human serum albumin in the composition are in a ratio by weight from about 1:95 to about 1:150. In some embodiments, the Docetaxel and the human serum albumin in the composition are in a ratio by weight from about 1:100 to about 1:150. In some embodiments, the Docetaxel and the human serum albumin in the composition are in a ratio by weight from about 1:80 to about 1:140. In some embodiments, the Docetaxel and the human serum albumin in the composition are in a ratio by weight from about 1:85 to about 1:140. In some embodiments, the Docetaxel and the human serum albumin in the composition are in a ratio by weight from about 1:90 to about 1:140. In some embodiments, the Docetaxel and the human serum albumin in the composition are in a ratio by weight from about 1:95 to about 1:140. In some embodiments, the Docetaxel and the human serum albumin in the composition are in a ratio by weight from about 1:80 to about 1:130. In some embodiments, the Docetaxel and the human serum albumin in the composition are in a ratio by weight from about 1:90 to about 1:130. In some embodiments, the Docetaxel and the human serum albumin in the composition are in a ratio by weight from about 1:95 to about 1:130. In some embodiments, the Docetaxel and the human serum albumin in the composition are in a ratio by weight from about 1:100 to about 1:130. In some embodiments, the Docetaxel and the human serum albumin in the composition are in a ratio by weight from about 1:85 to about 1:125. In some embodiments, the Docetaxel and the human serum albumin in the composition are in a ratio by weight from about 1:90 to about 1:125. In some embodiments, the Docetaxel and the human serum albumin in the composition are in a ratio by weight from about 1:95 to about 1:125. In some embodiments, the Docetaxel and the human serum albumin in the composition are in a ratio by weight from about 1:85 to about 1:120. In some embodiments, the Docetaxel and the human serum albumin in the composition are in a ratio by weight from about 1:90 to about 1:120. In some embodiments, the Docetaxel and the human serum albumin in the composition are in a ratio by weight from about 1:90 to about 1:110. In some embodiments, the Docetaxel and the human serum albumin in the composition are in a ratio by weight from about 1:95 to about 1:105. In some embodiments, the Docetaxel and the human serum albumin in the composition are in a ratio by weight of about 1:80, about 1:85, about 1:90, about 1:95, about 1:100, about 1:105, about 1:110, about 1:115, about 1:120, about 1:125, about 1:130, about 1:135, about 1:140, about 1:145, about 1:150, about 1:155, about 1:160, about 1:170, about 1:180, about 1:190, about 1:200, about 1:210, about 1:220, or about 1: 250. In some embodiments, the Docetaxel and the human serum albumin in the composition are in a ratio by weight of about 1:90. In some embodiments, the Docetaxel and the human serum albumin in the composition are in a ratio by weight of about 1:95. In some embodiments, the Docetaxel and the human serum albumin in the composition are in a ratio by weight of about 1:100. In some embodiments, the Docetaxel and the human serum albumin in the composition are in a ratio by weight of about 1:105. In some embodiments, the Docetaxel and the human serum albumin in the composition are in a ratio by weight of about 1:110. In some embodiments, the Docetaxel and the human serum albumin in the composition are in a ratio by weight of about 1:115. In some embodiments, the Docetaxel and the human serum albumin in the composition are in a ratio by weight of about 1:120.

In some embodiments, the composition comprises one amino acid. In some embodiments, the amino acid in the composition is aspartic acid. In some embodiments, the amino acid in the composition is glutamic acid. In some embodiments, the amino acid in the composition is cysteine. In some embodiments, the composition comprises two amino acids. In some embodiments, the composition comprises aspartic acid and glutamic acid. In some embodiments, the composition comprises two amino acids, wherein at least one amino acid in the composition is aspartic acid or glutamic acid. In some embodiments, the composition comprises two amino acids, wherein at least one amino acid in the composition is aspartic acid. In some embodiments, the composition comprises two amino acids, wherein at least one amino acid in the composition is glutamic acid. In some embodiments, the amino acid and the Docetaxel in the composition have a ratio by weight of no less than about 0.5:1. In some embodiments, the amino acid and the Docetaxel in the composition have a ratio by weight of no less than about 1:1. In some embodiments, the amino acid and the Docetaxel in the composition have a ratio by weight of no less than about 2:1. In some embodiments, the amino acid and the Docetaxel in the composition have a ratio by weight of about 2:1. In some embodiments, the amino acid and the Docetaxel in the composition have a ratio by weight of about 2:1.

In some embodiments, the composition further comprises a sugar alcohol or a sugar. In some embodiments, the composition further comprises a sugar alcohol. In some embodiments, the sugar alcohol in the composition is selected from mannitol, sorbitol, inositol, and xylitol. In some embodiments, the sugar alcohol in the composition is mannitol. In some embodiments, the sugar alcohol in the composition is sorbitol. In some embodiments, the sugar alcohol and the Docetaxel in the composition have a ratio by weight of no less than about 5:1. In some embodiments, the sugar alcohol and the Docetaxel in the composition have a ratio by weight of no less than about 10:1. In some embodiments, the sugar alcohol and the Docetaxel in the composition have a ratio by weight of no less than about 15:1. In some embodiments, the sugar alcohol and the Docetaxel in the composition have a ratio by weight of no less than about 20:1. In some embodiments, the sugar alcohol and the Docetaxel in the composition have a ratio by weight of no less than about 25:1. In some embodiments, the sugar alcohol and the Docetaxel in the composition have a ratio by weight of no less than about 50:1. In some embodiments, the sugar alcohol and the Docetaxel in the composition have a ratio by weight of no less than about 75:1. In some embodiments, the sugar alcohol and the Docetaxel in the composition have a ratio by weight of no less than about 100:1. In some embodiments, the sugar alcohol and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar alcohol and the Docetaxel in the composition have a ratio by weight of about 20:1. In some embodiments, mannitol and the Docetaxel in the composition have a ratio by weight of about 20:1.

In some embodiments, the composition further comprises a sugar. In some embodiments, the sugar in the composition is selected from glucose, sucrose, lactose, maltose, trehalose, fructose, hexose, and raffinose. In some embodiments, the sugar in the composition is glucose. In some embodiments, the sugar in the composition is lactose. In some embodiments, the sugar and the Docetaxel in the composition have a ratio by weight of no less than about 5:1. In some embodiments, the sugar and the Docetaxel in the composition have a ratio by weight of no less than about 10:1. In some embodiments, the sugar and the Docetaxel in the composition have a ratio by weight of no less than about 15:1. In some embodiments, the sugar and the Docetaxel in the composition have a ratio by weight of no less than about 20:1. In some embodiments, the sugar and the Docetaxel in the composition have a ratio by weight of no less than about 25:1. In some embodiments, the sugar and the Docetaxel in the composition have a ratio by weight of no less than about 50:1. In some embodiments, the sugar and the Docetaxel in the composition have a ratio by weight of no less than about 75:1. In some embodiments, the sugar and the Docetaxel in the composition have a ratio by weight of no less than about 100:1. In some embodiments, the sugar and the Docetaxel in the composition have a ratio by weight of about 5:1, about 10:1, about 15:1, about 18:1, about 20:1, about 22:1, about 25:1, about 30:1, about 35:1, about 40:1, about 50:1, about 75:1, or about 100:1. In some embodiments, the sugar and the Docetaxel in the composition have a ratio by weight of about 20:1. In some embodiments, glucose and the Docetaxel in the composition have a ratio by weight of about 20:1.

In some embodiments, the human serum albumin is a native human serum albumin. In some embodiments, the human serum albumin is a native human serum albumin obtained from pools of human plasma. In some embodiments, the human serum albumin is a recombinant human serum albumin. In some embodiments, the human serum albumin is a fatty acid free human serum albumin. In some embodiments, the human serum albumin is essentially fatty acid free.

In some embodiments, the amount of aqueous solvent in the first aqueous solution is from about 0.4 mL to about 5 mL per 100 mg of human serum albumin.

In some embodiments, the amount of aqueous solvent in the first aqueous solution is from about 0.75 mL to about 2 mL per 100 mg of human serum albumin.

In some embodiments, the amount of aqueous solvent in the first aqueous solution is about 1 mL per 100 mg of human serum albumin.

In some embodiments, the polar water-miscible organic solvent is an alcohol selected from the group consisting of ethanol, isopropanol, t-butanol, and mixtures thereof.

In some embodiments, the polar water-miscible organic solvent is selected from t-butanol, ethanol, and mixtures thereof.

In some embodiments, the polar water-miscible organic solvent is a mixture of t-butanol and ethanol. In some embodiments, the polar water-miscible organic solvent is a mixture of t-butanol and ethanol, in which the ratio of t-butanol and ethanol by volume is from about 4:1 to about 10:1. In some embodiments, the polar water-miscible organic solvent is a mixture of t-butanol and ethanol, in which the ratio of t-butanol and ethanol by volume is about 7:1.

In some embodiments, the polar water-miscible organic solvent comprises ethanol

In some embodiments, the polar water-miscible organic solvent is ethanol.

In some embodiments, the first aqueous solution contains t-butanol.

In some embodiments, the polar water-miscible organic solvent is acetone.

In some embodiments, the first aqueous solvent is water.

In some embodiments, the first aqueous solution contains t-butanol and ethanol.

In some embodiments, the adding is carried out dropwise.

In some embodiments, the adding the organic solution into the first aqueous solution is carried out at a temperature from about 0° C. to about 25° C.

In some embodiments, the mixing (or adding) is carried out at a temperature from about 0° C. to about 25° C. In some embodiments, the mixing (or adding) is carried out at a temperature from about 0° C. to about 15° C. In some embodiments, the mixing (or adding) is carried out at a temperature from about 0° C. to about 5° C. In some embodiments, the mixing is carried out at a temperature from about 5° C. to about 10° C. In some embodiments, the mixing (or adding) is carried out at a temperature from about 5° C. to about 15° C. In some embodiments, the mixing (or adding) is carried out at a temperature from about 0° C. to about 10° C.

In some embodiments, the second aqueous solution contains t-butanol.

In some embodiments, the second aqueous solution contains t-butanol and ethanol.

In some embodiments, the volume ratio of the amount of water to the amount of the polar organic solvent in the second aqueous solution is in a range from about 1:1 to about 1000:1. In some embodiments, the volume ratio of the amount of water to the amount of the polar organic solvent in the second aqueous solution is in a range from about 1.5:1 to about 100:1. In some embodiments, the volume ratio of the amount of water to the amount of the polar organic solvent in the second aqueous solution is in a range from about 1.5:1 to about 10:1. In some embodiments, the volume ratio of the amount of water to the amount of the polar organic solvent in the second aqueous solution is in a range from about 1.5:1 to about 5:1. In some embodiments, the volume ratio of the amount of water to the amount of the polar organic solvent in the second aqueous solution is in a range from about 2:1 to about 3:1. In some embodiments, the volume ratio of the amount of water to the amount of the polar organic solvent is in a range from about 1.5:1 to about 5:1. In some embodiments, the volume ratio of the amount of water to the amount of the polar organic solvent is in a range from about 2:1 to about 3:1. In some embodiments, the volume ratio of the amount of water to the amount of the polar organic solvent in the second aqueous solution is about 1.5:1, about 2:1, about 2.2:1, about 2.3:1, about 2.4:1, about 2.5:1, about 3:1, about 4:1, about 5:1, about 6:1, about 7:1, about 8:1, about 9:1, or about 10:1.

In some embodiments, the methods further comprise the step of removing the organic solvent (e.g. t-butanol, ethanol, and mixtures thereof) and the aqueous solvent (e.g., water) from the second aqueous solution to obtain the solid composition comprising Docetaxel, human serum albumin, and one or more amino acids selected from aspartic acid, glutamic acid, or cysteine, and a sugar alcohol or a sugar as described herein.

In some embodiments, the removing is carried out by lyophilization.

In some embodiments, the composition is a solid formulation.

In some embodiments, the composition is an aqueous formulation. In some embodiments, the aqueous formulation is substantially free of solvent other than water. In some embodiments, the aqueous formulation is free of a surfactant, which is selected from the group consisting of CREMOPHOR® surfactants and Polysorbate 80. In some embodiments, the aqueous formulation is a clear aqueous solution. In some embodiments, the aqueous formulation is a clear aqueous solution for at least 1 hour, at least 2 hours, at least 3 hours, at least 4 hours, at least 5 hours, at least 6 hours, at least 8 hours, or at least 24 hours.

In some embodiments, the concentration of Docetaxel in the aqueous formulation is from about 0.1 mg per 1 ml to about 1 mg per 1 ml of the aqueous solvent. In some embodiments, the concentration of Docetaxel in the aqueous formulation is from about 0.2 mg per 1 ml to about 0.4 mg per 1 ml of the aqueous solvent. In some embodiments, the concentration of Docetaxel in the aqueous formulation is about 0.2 mg per 1 ml of the aqueous solvent. In some embodiments, the concentration of Docetaxel in the aqueous formulation is about 0.25 mg per 1 ml of the aqueous solvent. In some embodiments, the concentration of Docetaxel in the aqueous formulation is about 0.3 mg per 1 ml of the aqueous solvent.

In some embodiments, the aqueous formulation is a clear aqueous solution, wherein the aqueous formulation is substantially free of solvent other than water, and wherein the concentration of Docetaxel in the aqueous formulation is from about 0.1 mg per 1 ml to about 1 mg per 1 ml of the aqueous solvent. In some embodiments, the aqueous formulation is a clear aqueous solution, wherein the aqueous formulation is substantially free of solvent other than water, and wherein the concentration of Docetaxel in the aqueous formulation is from about 0.2 mg per 1 ml to about 0.4 mg per 1 ml of the aqueous solvent. In some embodiments, the aqueous formulation is a clear aqueous solution, wherein the aqueous formulation is substantially free of solvent other than water, and wherein the concentration of Docetaxel in the aqueous formulation is about 0.2 mg per 1 ml, about 0.25 mg per 1 ml, about 0.3 mg per 1 ml, about 0.33 mg per 1 ml, or about 0.4 mg per 1 ml of the aqueous solvent.

In some embodiments, the present disclosure provides a pharmaceutical composition comprising the composition as prepared by a process as described herein, and a pharmaceutically acceptable carrier.

In some embodiments, the present disclosure provides a method of treating a cancer, the method comprising the step of administering to a subject in need thereof a therapeutically effective amount of the pharmaceutical composition comprising the composition as prepared by a process as described herein.

In some embodiments, the cancer is a solid tumor. In some embodiments, the cancer is selected from the group consisting of breast cancer, non-small cell lung cancer, prostate cancer, gastric cancer, head and neck cancer, ovarian cancer, pancreatic cancer, and Kaposi's sarcoma.

### EXAMPLES

### Example 1: Composition Comprising Docetaxel, Human Serum Albumin (HSA), and Amino Acids

(A) 4.5 ml of water; (B) 10 mg of arginine (Arg) dissolved in 4.5 ml of water; (C) 10 mg of aspartic acid (Asp) dissolved in 4.5 ml of water: (D) 10 mg of cysteine (Cys) dissolved in 4.5 ml of water; (E) 10 mg of glutamic acid (Glu) dissolved in 4.5 ml of water,; (F) 10 mg of lysine (Lys) dissolved in 4.5 ml of water; or (G) 10 mg of proline (Pro) dissolved in 4.5 ml of water were added to 7 different round bottom flasks, each with 2.5 ml of 20% Human Albumin solution for infusion (500 mg HSA), respectively. (Note: all amino acids used in the example section (all examples) are L-amino acids) After the flasks were put in an ice bath, docetaxel (5 mg) dissolved in a mixed solvent (2.5 ml t-butanol and 0.5 ml ethanol) was added into each of the 7 flasks dropwise with stirring. Upon completion of the addition, the clear solutions were obtained. The resulting clear aqueous solutions were lyophilized overnight to give white solids.

35 mg of the lyophilized solids from each of 7 flasks were reconstituted by adding 1 mL water all to give the clear solutions. To 300 μl of each reconstituted solution was added 700 μl of acetonitrile. The mixtures were vortexed for seconds and then centrifuged at 4,000 g for 5 minutes. The supernatants were removed and collected followed by injection on HPLC. The data of HPLC of the percent amount of 7-Epi-docetaxel relative to docetaxel in the each sample were shown in table 1.

35 mg of the lyophilized solids from each of 7 flasks were heated at 50° C. for 5 hours, and then were reconstituted by adding 1 mL water all to give clear solutions. To 300 μl of each reconstituted solution was added 700 μl of acetonitrile. The mixtures were vortexed for seconds and then centrifuged at 4,000 g for 5 minutes. The supernatants were removed and collected followed by injection on HPLC. The data of HPLC of the percent amount of 7-Epi-docetaxel relative to docetaxel in the each sample were shown in table 1.

The data in the table 1 illustrated that adding Asp or Glu significantly decreased the formation of 7-Epi-docetaxel in the composition, and adding Arg or Lys significantly increased the formation of 7-Epi-docetaxel in the composition, while adding Cys or Pro slightly decreased the formation of 7-Epi-docetaxel in the composition.

TABLE 1

| | Amino acid (AA) | | | | | | |
|---|---|---|---|---|---|---|---|
| | No amino | | | | | | |
| | acid | Arg | Asp | Cys | Glu | Lys | Pro |
| At room | 0.253% | 1.201% | 0.085% | 0.167% | 0.095% | 1.755% | 0.248% |
| temperature | | | | | | | |
| Heated at 50° C. | 1.822% | 4.212% | 0.755% | 1.164% | 0.676% | 4.563% | 1.645% |
| for 5 hours | | | | | | | |

### Example 2: Composition Comprising Docetaxel, Human Serum Albumin (HSA), and Amino Acids

(A) 4.5 ml of water; (B) 10 mg of alanine (Ala) dissolved in 4.5 ml of water; (C) 10 mg of asparagine (Asn) dissolved in 4.5 ml of water, (D) 10 mg of HCl salt of cysteine (Cys.HCl) dissolved in 4.5 ml of water; (E) 10 mg of glycine (Gly) dissolved in 4.5 ml of water; (F) 10 mg of histidine (His) dissolved in 4.5 ml of water, or (G) 10 mg of serine (Ser) dissolved in 4.5 ml of water were added to 7 different round bottom flasks, each with 2.5 ml of 20% Human Albumin solution for infusion (500 mg HSA), respectively. After the flasks were put in an ice bath, docetaxel (5 mg) dissolved in a mixed solvent (2.5 ml t-butanol and 0.5 ml ethanol) was added into each of the 7 flasks dropwise with stirring. Upon completion of the addition, the clear solutions were obtained. The resulting clear aqueous solutions were lyophilized overnight to give white solids.

35 mg of the lyophilized solids from each of 7 flasks were reconstituted by adding 1 mL water all to give the clear solutions. To 300 μl of each reconstituted solution was added 700 μl of acetonitrile. The mixtures were vortexed for seconds and then centrifuged at 4,000 g for 5 minutes. The supernatants were removed and collected followed by injection on HPLC. The data of HPLC of the percent amount of 7-Epi-docetaxel relative to docetaxel in the each sample were shown in table 2.

35 mg of the lyophilized solids from each of 7 flasks were heated at 50° C. for 4 hours, and then were reconstituted by adding 1 mL water all to give clear solutions. To 300 μl of each reconstituted solution was added 700 μl of acetonitrile. The mixtures were vortexed for seconds and then centrifuged at 4,000 g for 5 minutes. The supernatants were removed and collected followed by injection on HPLC. The data of HPLC of the percent amount of 7-Epi-docetaxel relative to docetaxel in the each sample were shown in table 2.

The data in the table 2 illustrated that adding Cys.HCl decreased the formation of 7-Epi-docetaxel in the composition, while adding Ala, Asn, Gly, His, or Ser didn't change much the amount of formation of 7-Epi-docetaxel in the composition.

TABLE 2

| | Amino acid (AA) | | | | | | |
|---|---|---|---|---|---|---|---|
| | No amino | | | | | | |
| | acid | Ala | Asn | Cys · HCl | Gly | His | Ser |
| At room | 0.219% | 0.219% | 0.215% | 0.106% | 0.263% | 0.289% | 0.266% |
| temperature | | | | | | | |
| Heated at 50° C. | 2.17% | 1.426% | 1.749% | 0.716% | 2.124% | 2.035% | 1.592% |
| for 4 hours | | | | | | | |

### Example 3: Composition Comprising Docetaxel, Human Serum Albumin (HSA), Amino Acids, and Sugar Alcohol

(A) 10 mg of aspartic acid (Asp) and 1 mg of HCl salt of cysteine (Cys.HCl) dissolved in 4.5 ml of water; (B) 10 mg of aspartic acid (Asp), 1 mg of HCl salt of cysteine (Cys.HCl), and 25 mg of mannitol dissolved in 4.5 ml of water; (C) 10 mg of aspartic acid (Asp), 1 mg of HCl salt of cysteine (Cys.HCl), and 50 mg of mannitol dissolved in 4.5 ml of water; or (D) 10 mg of aspartic acid (Asp), 1 mg of HCl salt of cysteine (Cys.HCl), and 100 mg of mannitol dissolved in 4.5 ml of water were added to 4 different round bottom flasks, each with 2.5 ml of 20% Human Albumin solution for infusion (500 mg HSA), respectively. After the flasks were put in an ice and water bath at about 10° C., docetaxel (5 mg) dissolved in a mixed solvent (2.5 ml t-butanol and 0.5 ml ethanol) was added into each of the 4 flasks dropwise with stirring. Upon completion of the addition, the clear solutions were obtained. The resulting clear aqueous solutions were lyophilized overnight to give white solids.

35 mg of the lyophilized solids from each of 4 flasks were reconstituted by adding 1 mL water all to give the clear solutions. To 300 μl of each reconstituted solution was added 700 μl of acetonitrile. The mixtures were vortexed for seconds and then centrifuged at 4,000 g for 5 minutes. The supernatants were removed and collected followed by injection on HPLC. The data of HPLC of the percent amount of 7-Epi-docetaxel relative to docetaxel in the each sample were shown in table 3.

The data in the table 3 illustrated that adding mannitol into the composition decreased the formation of 7-Epi-docetaxel in the composition.

TABLE 3

| | Sugar Alcohol | | | |
|---|---|---|---|---|
| | 0 mg | 25 mg | 50 mg | 100 mg |
| | mannitol | mannitol | mannitol | mannitol |
| Percent amount | 0.160% | 0.107% | 0.070% | 0.035% |
| of 7-Epi-docetaxel | | | | |

### Example 4: Composition Comprising Docetaxel, Human Serum Albumin (HSA), and Amino Acids

(A) 4.5 ml of water; (B) 2 mg of Aspartic acid (Asp) dissolved in 4.5 ml of water; (C) 5 mg of aspartic acid (Asp) dissolved in 4.5 ml of water; (D) 10 mg of aspartic acid (Asp) dissolved in 4.5 ml of water; (E) 2 mg of Glutamic acid (Glu) dissolved in 4.5 ml of water; (F) 5 mg of glutamic acid (Glu) dissolved in 4.5 ml of water; or (G) 10 mg of glutamic acid (Glu) dissolved in 4.5 ml of water were added to 7 different round bottom flasks, each with 2.5 ml of 20% Human Albumin solution for infusion (500 mg HSA), respectively. After the flasks were put in an ice bath, docetaxel (5 mg) dissolved in a mixed solvent (2.5 ml t-butanol and 0.5 ml ethanol) was added into each of the 7 flasks dropwise with stirring. Upon completion of the addition, the clear solutions were obtained. The resulting clear aqueous solutions were lyophilized overnight to give white solids.

35 mg of the lyophilized solids from each of 7 flasks were reconstituted by adding 1 mL water all to give the clear solutions. To 300 μl of each reconstituted solution was added 700 μl of acetonitrile. The mixtures were vortexed for seconds and then centrifuged at 4,000 g for 5 minutes. The supernatants were removed and collected followed by injection on HPLC. The data of HPLC of the percent amount of 7-Epi-docetaxel relative to docetaxel in the each sample were shown in table 4.

The data in the table 4 illustrated that adding Asp or Glu in the composition decreased the formation of 7-Epi-docetaxel in the composition, and adding more amount of Asp or Glu in the composition further decreased the formation of 7-Epi-docetaxel in the composition.

TABLE 4

| | Amino acid (AA) | | | | | | |
|---|---|---|---|---|---|---|---|
| | AA | Asp | Asp | Asp | Glu | Glu | Glu |
| | 0 mg | 2 mg | 5 mg | 10 mg | 2 mg | 5 mg | 10 mg |
| Percent amount of | 0.277% | 0.189% | 0.102% | 0.092% | 0.235% | 0.151% | 0.106% |
| 7-Epi-docetaxel | | | | | | | |

### Example 5: Composition Comprising Docetaxel, Human Serum Albumin (HSA), Amino Acid, and Sugar Alcohol or Sugar

(A) 10 mg of aspartic acid (Asp) dissolved in 2.5 ml of water, (B) 10 mg of aspartic acid (Asp) and 25 mg of mannitol dissolved in 2.5 ml of water, (C) 10 mg of aspartic acid (Asp) and 50 mg of mannitol dissolved in 2.5 ml of water, (D) 10 mg of aspartic acid (Asp) and 100 mg of mannitol dissolved in 2.5 ml of water, (E) 10 mg of aspartic acid (Asp) and 25 mg of glucose dissolved in 2.5 ml of water, (F) 10 mg of aspartic acid (Asp) and 50 mg of glucose dissolved in 2.5 ml of water, or (G) 10 mg of aspartic acid (Asp) and 100 mg of glucose dissolved in 2.5 ml of water were added to 7 different round bottom flasks, each with 2.5 ml of 20% Human Albumin solution for infusion (500 mg HSA), respectively. After the flasks were put in an ice and water bath at about 10° C., docetaxel (5 mg) dissolved in a mixed solvent (1.75 ml t-butanol and 0.25 ml ethanol) was added into each of the 7 flasks dropwise with stirring. Upon completion of the addition, the clear solutions were obtained. The resulting clear aqueous solutions were lyophilized overnight to give white solids.

35 mg of the lyophilized solids from each of 7 flasks were reconstituted by adding 1 mL water all to give the clear solutions. To 300 μl of each reconstituted solution was added 700 μl of acetonitrile. The mixtures were vortexed for seconds and then centrifuged at 4,000 g for 5 minutes. The supernatants were removed and collected followed by injection on HPLC. The data of HPLC of the percent amount of 7-Epi-docetaxel relative to docetaxel in the each sample were shown in table 5.

35 mg of the lyophilized solids from each of 7 flasks were heated at 40° C. for 4 hours, and then were reconstituted by adding 1 mL water all to give clear solutions. To 300 μl of each reconstituted solution was added 700 μl of acetonitrile. The mixtures were vortexed for seconds and then centrifuged at 4,000 g for 5 minutes. The supernatants were removed and collected followed by injection on HPLC. The data of HPLC of the percent amount of 7-Epi-docetaxel relative to docetaxel in the each sample were shown in table 5.

The data in the table 5 illustrated that adding mannitol or glucose into the composition decreased the formation of 7-Epi-docetaxel in the composition, and adding more amount of mannitol or glucose into the composition further decreased the amount of the formation of 7-Epi-docetaxel in the composition.

TABLE 5

| | Sugar alcohol or Sugar | | | | | | |
|---|---|---|---|---|---|---|---|
| | | Mannitol | Mannitol | Mannitol | Glucose | Glucose | Glucose |
| | none | 25 mg | 50 mg | 100 mg | 25 mg | 50 mg | 100 mg |
| At room | 0.086% | 0.042% | 0.054% | 0.024% | 0.066% | 0.044% | 0.031% |
| temperature | | | | | | | |
| Heated at | 0.505% | 0.276% | 0.303% | 0.137% | 0.308% | 0.187% | 0.152% |
| 40° C. for 4 | | | | | | | |
| hours | | | | | | | |

### Example 6: Measure pH Value of the Clear Aqueous Solution of Composition Comprising Docetaxel, Human Serum Albumin (HSA), Amino Acid, and Sugar Alcohol

40 mg of aspartic acid (Asp) and 400 mg of mannitol dissolved in 10 ml of water was added to a round bottom flasks with 10 ml of 20% Human Albumin solution for infusion (2 g HSA). After the flask was put into an ice bath, docetaxel (20 mg) dissolved in a mixed solvent of 7 ml t-butanol and 1 ml ethanol was added into the flask dropwise with stirring. Upon completion of the addition, the clear solutions were obtained. The resulting clear aqueous solution was kept at about 9° C. for about 4.5 hours, and filtered by a 0.22 micron aqueous phase filter. The filtered solution was divided evenly into 8 glass vials and lyophilized overnight to give white solids.

10 ml of 0.9% saline was added into one glass vial with the lyophilized product to give a clear aqueous solution. The clear aqueous solution was kept at about 25° C. and measured for pH value. The pH value of the clear aqueous solution is 6.12 (3 measurements: 6.11, 6.13, and 6.11).

10 ml of 5% dextrose solution was added into one glass vial with the lyophilized product to give a clear aqueous solution. The clear aqueous solution was kept at about 25° C. and measured for pH value. The pH value of the clear aqueous solution is 5.90 (3 measurements: 5.89, 5.91, and 5.91).

5 ml of 0.9% saline and 5 ml of 5% dextrose solution were mixed and added into one glass vial with the lyophilized product to give a clear aqueous solution. The clear aqueous solution was kept at about 25° C. and measured for pH value. The pH value of the clear aqueous solution is 6.01 (3 measurements: 6.01, 6.01, and 6.01).

### Example 7: Composition Comprising Docetaxel, Human Serum Albumin (HSA), Amino Acid, and Sugar Alcohol

60 mg of aspartic acid (Asp) and 600 mg of mannitol dissolved in 15 ml of water was added to a round bottom flasks with 15 ml of 20% Human Albumin solution for infusion (3 g HSA). After the flask was put into an ice bath, docetaxel (30 mg) dissolved in a mixed solvent of 10.5 ml t-butanol and 1.5 ml ethanol was added into the flask dropwise with stirring. Upon completion of the addition, the clear solutions were obtained. The resulting clear aqueous solution was kept at about 9° C. for about 4 hours, and filtered by a 0.22 micron aqueous phase filter. The filtered solution was divided evenly into 6 glass vials and lyophilized overnight to give white solids.

20 ml of 0.9% saline was added into one glass vial with the lyophilized product to give a clear aqueous solution.

### Example 8: Measure the Correlation Between HPLC Peak Area and the Docetaxel Concentration

Methanol solutions of Docetaxel in 6 different concentrations, 0.0125 mg/ml, 0.025 mg/ml, 0.05 mg/ml, 0.075 mg/ml, 0.1 mg/ml, and 0.125 mg/ml, were prepared. The 6 Docetaxel methanol solutions were analyzed in HPLC. The peak area and concentration of Docetaxel were correlated using linear regression. The linear regression data is shown as below.

Y (peak area)=−8459+2.798E7*X (concentration), R=0.9999, P<0.0001.

### Example 9: Measure the Docetaxel Concentrations in the Clear Aqueous Solutions Before the Filtration at 0 Hour, and After the Filtration at 1 Hour, 2 Hours, 3 Hours, and 4 Hours

Two of glass vials with the lyophilized product from Example 7 were added with 20 ml of 0.9% saline in each vial. Immediately after the lyophilized solids in both vials were dissolved, the aqueous solution of the two vials were combined, and 1 ml of the aqueous solution was taken out from the 40 ml solution. Then 1 ml of the solution was named as the solution DC-0-0h. To 300 μl of the solution DC-0-0h was added 700 μl of acetonitrile. The mixture was vortexed for seconds and then centrifuged at 4,000 g for 5 minutes. The supernatants were removed and collected followed by injection on HPLC. The same procedure was repeated 2 more times for the solution DC-0-0h. Based on the HPLC data and the measurement data of Example 8, the Docetaxel concentrations of the supernatants prepared from solution of DC-0-0h have been calculated and shown in the Table 6.

TABLE 6

| | Docetaxel | Average Docetaxel |
|---|---|---|
| Solution | Concentration | Concentration |
| Number | (mg/ml) | (mg/ml) |
| DC-0-0h-1 | 0.07040 | 0.07102 |
| DC-0-0h-2 | 0.07126 | |
| DC-0-0h-3 | 0.07140 | |

At 1 hour, additional 5 ml of the aqueous solution was taken out from the remaining aqueous solution in the vial. Then 1 ml of the solution was taken out from the 5 ml aqueous solution and filtered by a 0.22 micron aqueous phase filter to give the solution DC-1-1h, and the remaining 4 ml of the solution was filtered by the same 0.22 micron aqueous phase filter at 1 ml at a time to give the solutions DC-2-1h, DC-3-1h, DC-4-1h, and DC-5-1h. To 300 μl of the solution DC-5-1h was added 700 μl of acetonitrile. The mixture was vortexed for seconds and then centrifuged at 4,000 g for 5 minutes. The supernatant was removed and collected followed by injection on HPLC. The same procedure was repeated 2 more times for the solution DC-5-1h. Based on the HPLC data and the measurement data of Example 8, the Docetaxel concentrations of the supernatants prepared from solution of DC-5-1h have been calculated and shown in the Table 7. At 1 hour, the Docetaxel concentration of the aqueous solution after the filtration was about 99.69% of the Docetaxel concentration of the aqueous solution at 0 hour before the filtration.

TABLE 7

| | Docetaxel | Average Docetaxel |
|---|---|---|
| Solution | Concentration | Concentration |
| Number | (mg/ml) | (mg/ml) |
| DC-5-1h-1 | 0.07027 | 0.07080 |
| DC-5-1h-2 | 0.07128 | |
| DC-5-1h-3 | 0.07086 | |

At 2 hour, 5 ml of the aqueous solution was taken out from the remaining aqueous solution in the vial. The experiments were done for the 5 ml of the aqueous solution taken out at 2 hour using the same protocol as for the 5 ml of the aqueous solution taken out at 1 hour. Based on the HPLC data and the measurement data of Example 8, the Docetaxel concentrations of the supernatants prepared from solution of DC-5-2h have been calculated and shown in the Table 8. At 2 hour, the Docetaxel concentration of the aqueous solution after the filtration was about 98.72% of the Docetaxel concentration of the aqueous solution at 0 hour before the filtration.

TABLE 8

| | Docetaxel | Average Docetaxel |
|---|---|---|
| Solution | Concentration | Concentration |
| Number | (mg/ml) | (mg/ml) |
| DC-5-2h-1 | 0.07001 | 0.07011 |
| DC-5-2h-2 | 0.07053 | |
| DC-5-2h-3 | 0.06979 | |

At 3 hour, 5 ml of the aqueous solution was taken out from the remaining aqueous solution in the vial. The experiments were done for the 5 ml of the aqueous solution taken out at 3 hour using the same protocol as for the 5 ml of the aqueous solution taken out at 1 hour. Based on the HPLC data and the measurement data of Example 8, the Docetaxel concentrations of the supernatants prepared from solution of DC-5-3h have been calculated and shown in the Table 9. At 3 hour, the Docetaxel concentration of the aqueous solution after the filtration was about 91.40% of the Docetaxel concentration of the aqueous solution at 0 hour before the filtration.

TABLE 9

| | Docetaxel | Average Docetaxel |
|---|---|---|
| Solution | Concentration | Concentration |
| Number | (mg/ml) | (mg/ml) |
| DC-5-3h-1 | 0.06491 | 0.06491 |
| DC-5-3h-2 | 0.06471 | |
| DC-5-3h-3 | 0.06512 | |

At 4 hour, 5 ml of the aqueous solution was taken out from the remaining aqueous solution in the vial. The experiments were done for the 5 ml of the aqueous solution taken out at 4 hour using the same protocol as for the 5 ml of the aqueous solution taken out at 1 hour. Based on the HPLC data and the measurement data of Example 8, the Docetaxel concentrations of the supernatants prepared from solution of DC-5-4h have been calculated and shown in the Table 10. At 4 hour, the Docetaxel concentration of the aqueous solution after the filtration was about 72.42% of the Docetaxel concentration of the aqueous solution at 0 hour before the filtration.

TABLE 10

| | Docetaxel | Average Docetaxel |
|---|---|---|
| Solution | Concentration | Concentration |
| Number | (mg/ml) | (mg/ml) |
| DC-5-4h-1 | 0.05123 | 0.05143 |
| DC-5-4h-2 | 0.05147 | |
| DC-5-4h-3 | 0.05160 | |

### Example 10: Composition Comprising Docetaxel, Human Serum Albumin (HSA), Amino Acids, and Sugar Alcohol

(A) 1.2012 g of mannitol dissolved in 30 ml of water was added to a round bottom flask with 30 ml of 20% Human Albumin solution for infusion (6 g HSA). After the flask was put in an ice bath at about 6-8° C., docetaxel (59.8 mg) dissolved in a mixed solvent (21 ml t-butanol and 3 ml ethanol) was added into the flask dropwise with stirring. Upon completion of the addition, the clear solutions were obtained. The resulting clear aqueous solution was divided evenly into 6 glass vials (each with about 13.9 ml) and lyophilized overnight to give white solids.

(B) 119.8 mg of arginine (Arg) and 1.2018 g of mannitol dissolved in 30 ml of water was added to a round bottom flask with 30 ml of 20% Human Albumin solution for infusion (6 g HSA). After the flask was put in an ice bath at about 6-8° C., docetaxel (59.8 mg) dissolved in a mixed solvent (21 ml t-butanol and 3 ml ethanol) was added into the flask dropwise with stirring. Upon completion of the addition, the clear solutions were obtained. The resulting clear aqueous solution was divided evenly into 6 glass vials (each with about 13.9 ml) and lyophilized overnight to give white solids.

(C) 120.5 mg of aspartic acid (Asp) and 1.2001 g of mannitol dissolved in 30 ml of water was added to a round bottom flask with 30 ml of 20% Human Albumin solution for infusion (6 g HSA). After the flask was put in an ice bath at about 6-8° C., docetaxel (60.3 mg) dissolved in a mixed solvent (21 ml t-butanol and 3 ml ethanol) was added into the flask dropwise with stirring. Upon completion of the addition, the clear solutions were obtained. The resulting clear aqueous solution was divided evenly into 6 glass vials (each with about 13.9 ml) and lyophilized overnight to give white solids.

(D) 119.9 mg of HCl salt of cysteine (Cys.HCl) and 1.1991 g of mannitol dissolved in 30 ml of water was added to a round bottom flask with 30 ml of 20% Human Albumin solution for infusion (6 g HSA). After the flask was put in an ice bath at about 6-8° C., docetaxel (59.9 mg) dissolved in a mixed solvent (21 ml t-butanol and 3 ml ethanol) was added into the flask dropwise with stirring. Upon completion of the addition, the clear solutions were obtained. The resulting clear aqueous solution was divided evenly into 6 glass vials (each with about 13.9 ml) and lyophilized overnight to give white solids.

(E) 119.7 mg of glutamic acid (Glu) and 1.2006 g of mannitol dissolved in 30 ml of water was added to a round bottom flask with 30 ml of 20% Human Albumin solution for infusion (6 g HSA). After the flask was put in an ice bath at about 6-8° C., docetaxel (59.8 mg) dissolved in a mixed solvent (21 ml t-butanol and 3 ml ethanol) was added into the flask dropwise with stirring. Upon completion of the addition, the clear solutions were obtained. The resulting clear aqueous solution was divided evenly into 6 glass vials (each with about 13.9 ml) and lyophilized overnight to give white solids.

(F) 120.3 mg of HCl salt of lysine (Lys.HCl) and 1.1996 g of mannitol dissolved in 30 ml of water was added to a round bottom flask with 30 ml of 20% Human Albumin solution for infusion (6 g HSA). After the flask was put in an ice bath at about 6-8° C., docetaxel (60.3 mg) dissolved in a mixed solvent (21 ml t-butanol and 3 ml ethanol) was added into the flask dropwise with stirring. Upon completion of the addition, the clear solutions were obtained. The resulting clear aqueous solution was divided evenly into 6 glass vials (each with about 13.9 ml) and lyophilized overnight to give white solids.

(G) 120.9 mg of proline (Pro) and 1.1998 g of mannitol dissolved in 30 ml of water was added to a round bottom flask with 30 ml of 20% Human Albumin solution for infusion (6 g HSA). After the flask was put in an ice bath at about 6-8° C., docetaxel (60.2 mg) dissolved in a mixed solvent (21 ml t-butanol and 3 ml ethanol) was added into the flask dropwise with stirring. Upon completion of the addition, the clear solutions were obtained. The resulting clear aqueous solution was divided evenly into 6 glass vials (each with about 13.9 ml) and lyophilized overnight to give white solids.

One vial of the lyophilized products from each of the above 7 experiments (six with different amino acids and one without amino acid) was added with 2.5 mL water, and followed by adding 7.5 ml acetonitrile. The mixtures were vortexed for 3 minutes, then centrifuged, and filtered. 1 ml of the filtered solutions was diluted by 99 ml of acetonitrile and followed by injection on HPLC. The data of HPLC of the percent amount of 7-Epi-docetaxel relative to docetaxel in the each experiment were shown in table 11.

One vial of the lyophilized products from each of the above 7 experiments (six with different amino acids and one without amino acid) was heated at 40° C. for 1 day, and then added with 2.5 mL water into the vials, and followed by adding 7.5 ml acetonitrile. The mixtures were vortexed for 3 minutes, then centrifuged, and filtered. 1 ml of the filtered solutions was diluted by 99 ml of acetonitrile and followed by injection on HPLC. The data of HPLC of the percent amount of 7-Epi-docetaxel relative to docetaxel in the each experiment were shown in table 11.

The data in the table 11 illustrated that adding Asp or Glu into the composition significantly decreased the formation of 7-Epi-docetaxel in the composition, while adding Arg into the composition significantly increased the formation of 7-Epi-docetaxel in the composition.

TABLE 11

| | Amino acid (AA) | | | | | | |
|---|---|---|---|---|---|---|---|
| | No amino | | | | | | |
| | acid | Arg | Asp | Cys · HCl | Glu | Lys · HCl | Pro |
| At room | 0.35% | 1.89% | 0.07% | 0.10% | 0.07% | 0.02% | 0.22% |
| temperature | | | | | | | |
| Heated at 40° C. | 0.86% | 2.62% | 0.29% | 0.77% | 0.25% | 0.63% | 0.70% |
| for 1 day | | | | | | | |

### Example 11: Composition Comprising Docetaxel, Human Serum Albumin (HSA), Amino Acids, and Sugar Alcohol using t-butanol and Ethanol as Solvents

60.3 mg of aspartic acid (Asp) was dissolved in 15ml of water in a round bottom flask, and then 600.1 mg of mannitol was added into the flask. Then 15 ml of 20% Human Albumin solution for infusion (3 g HSA) was added into the flask with stirring. After the flask was put in an ice bath at about 8-10° C., 10.5 ml of t-butanol was added into the flask with rapidly stirring. Then docetaxel (30 mg) dissolved in 1.5 ml ethanol was added into the flask with rapidly stirring (The addition of docetaxel in ethanol took about 10 seconds). Upon completion of the addition, a slightly yellow clear solution was obtained, and filtered by a 0.22 micron PES filter. The resulting clear solution was clear with no precipitation for 3 hours. After 3 hours, the clear solution was added into 10 glass vials (each with about 2.5 ml) and lyophilized overnight to give the white solid product. One vial of the lyophilized product was added with 7 ml of 0.9% saline to give a clear solution, which stayed clear with no precipitation for 1 hour, and became cloudy in 2 hours.

7 ml of 0.9% saline was added into one glass vial with the lyophilized product to give a clear aqueous solution. The clear solution was measured for pH value. The pH value is 5.43 (3 measurements: 5.45, 5.42, and 5.42).

### Example 12: Composition Comprising Docetaxel, Human Serum Albumin (HSA), Amino Acids, and Sugar Alcohol using only Ethanol as Solvent

60.1 mg of aspartic acid (Asp) was dissolved in 15 ml of water in a round bottom flask, and then 600.2 mg of mannitol was added. Then 15 ml of 20% Human Albumin solution for infusion (3 g HSA) was added into the flask with stirring. After the flask was put in an ice bath at about 8-10° C., 10.5 ml of ethanol was added into the flask with rapidly stirring. Then docetaxel (30.1 mg) dissolved in 1.5 ml ethanol was added into the flask with rapidly stirring (The addition of docetaxel in ethanol took about 10 seconds). Upon completion of the addition, a slightly yellow clear solution was obtained, and filtered by a 0.22 micron PES filter. The resulting clear solution became cloudy with a large amount of precipitations within 1 hour.

### Example 13: Composition Comprising Docetaxel, Human Serum Albumin (HSA), Amino Acids, and Sugar Alcohol

(A) 100.3 mg of aspartic acid (Asp) was dissolved in 25 ml of water at 40° C. and cooled to about 6-10° C. Then 25 ml of 20% Human Albumin solution (5 g HSA) was addeded into this solution of Asp with stirring. At the same time, docetaxel (49.8 mg) was dissolved in 2.5 ml of ethanol, followed by adding 17.5 ml of t-butanol. After mixed well, the docetaxel solution was added into the solution of Asp and HSA with stirring at about 6-10° C. to give a clear solution. In the next step, the resulting clear solution was divided into 50 ml-size glass vials (each with about 13.9 ml) and lyophilized to give white solids.

(B) 100.3 mg of aspartic acid (Asp) and 500.3 mg of mannitol were dissolved in 25 ml of water at 40° C. and cooled to about 6-10° C. Then 25 ml of 20% Human Albumin solution for infusion (5 g HSA) was addeded into this solution of Asp with stirring. At the same time, docetaxel (50.0 mg) was dissolved in 2.5 ml of ethanol, followed by adding 17.5 ml of t-butanol. After mixed well, the docetaxel solution was added into the solution of Asp, mannitol, and HSA with stirring at about 6-10° C. to give a clear solution. In the next step, the resulting clear solution was divided into 50 ml-size glass vials (each with about 13.9 ml) and lyophilized to give white solids.

(C) 100.5 mg of aspartic acid (Asp) and 749.6 mg of mannitol were dissolved in 25 ml of water at 40° C. and cooled to about 6-10° C. Then 25 ml of 20% Human Albumin solution for infusion (5 g HSA) was addeded into this solution of Asp with stirring. At the same time, docetaxel (49.8 mg) was dissolved in 2.5 ml of ethanol, followed by adding 17.5 ml of t-butanol. After mixed well, the docetaxel solution was added into the solution of Asp, mannitol, and HSA with stirring at about 6-10° C. to give a clear solution. In the next step, the resulting clear solution was divided into 50 ml-size glass vials (each with about 13.9 ml) and lyophilized to give white solids.

(D) 200.4 mg of aspartic acid (Asp) and 2001.4 mg of mannitol were dissolved in 50 ml of water at 40° C. and cooled to about 6-10° C. Then 50 ml of 20% Human Albumin solution for infusion (10 g HSA) was addeded into this solution of Asp with stirring. At the same time, docetaxel (100.2 mg) was dissolved in 5 ml of ethanol, followed by adding 35 ml of t-butanol. After mixed well, the docetaxel solution was added into the solution of Asp, mannitol, and HSA with stirring at about 6-10° C. to give a clear solution. In the next step, the resulting clear solution was divided into 50 ml-size glass vials (each with about 13.9 ml) and lyophilized to give white solids.

(E) 100.1 mg of aspartic acid (Asp) and 1249.9 mg of mannitol were dissolved in 25 ml of water at 40° C. and cooled to about 6-10° C. Then 25 ml of 20% Human Albumin solution for infusion (5 g HSA) was addeded into this solution of Asp with stirring. At the same time, docetaxel (49.8 mg) was dissolved in 2.5 ml of ethanol, followed by adding 17.5 ml of t-butanol. After mixed well, the docetaxel solution was added into the solution of Asp, mannitol, and HSA with stirring at about 6-10° C. to give a clear solution. In the next step, the resulting clear solution was divided into 50 ml-size glass vials (each with about 13.9 ml) and lyophilized to give white solids.

One vial of the lyophilized products from each of the above 5 experiments (4 with different amount of mannitol and one without mannitol) was added with 2.5 mL water, and followed by adding 7.5 ml acetonitrile. The mixtures were vortexed for 3 minutes, then centrifuged, and filtered. 1 ml of the filtered solutions was diluted by 99 ml of acetonitrile and followed by injection on HPLC. The data of HPLC of the percent amount of 7-Epi-docetaxel relative to docetaxel in the each experiment were shown in table 12.

One vial of the lyophilized products from each of the above 5 experiments (4 with different amount of mannitol and one without mannitol) was heated at 40° C. for 1 day, and then added with 2.5 mL water into the vials, and followed by adding 7.5 ml acetonitrile. The mixtures were vortexed for 3 minutes, then centrifuged, and filtered. 1 ml of the filtered solutions was diluted by 99 ml of acetonitrile and followed by injection on HPLC. The data of HPLC of the percent amount of 7-Epi-docetaxel relative to docetaxel in the each experiment were shown in table 12.

The data in the table 12 illustrated that adding mannitol and adding more amount of mannitol into the composition significantly decreased the formation of 7-Epi-docetaxel in the composition.

TABLE 12

| | The ratio (mannitol/docetaxel) | | | | |
|---|---|---|---|---|---|
| | 0 | 10 | 15 | 20 | 25 |
| At room temperature | 0.189% | 0.118% | 0.104% | 0.074% | 0.066% |
| Heated at 40° C. for | 0.644% | 0.376% | 0.342% | 0.236% | 0.223% |
| 1 day | | | | | |

### Example 14: Scale-Up Preperation of Composition Comprising Docetaxel, Human Serum Albumin (HSA), Amino Acids, and Sugar Alcohol

| | | |
|---|---|---|
| Ingredient | Amount in one vial | Amount in 110 vials |

| | | | | |
|---|---|---|---|---|
| docetaxel | 10 | mg | 1.1 | g |
| ethanol | 0.5 | ml | 55 | ml (43.45 g) |
| t-butanol | 3.5 | ml | 385 | ml (300.685 g) |
| aspartic acid | 20 | mg | 2.2 | g |
| mannitol | 200 | mg | 22 | g |
| huamn albumin | 5 | ml | 550 | ml (588.5 g) |
| solution (20%) | | | | |
| water | 5 | ml | 550 | ml (550 g) |

Docetaxel (1.1021 g) was dissolved in 43.50 g of ethanol, and then mixed with 300.72 g of t-butanol. 2.2010 g of aspartic acid (Asp) was dissolved in 550 g of water at 50° C. and cooled to about 25° C., and then 22.0062 g of mannitol was added and dissolved in this aqueous solution. Then 588.47 g of 20% Human Albumin solution was added into the aqueous solution of aspartic acid and mannitol with stirring at 6-10° C., and followed by adding the previously prepared docetaxel (1.1021 g) solution. The solution was stirred for additional 10 minutes. Then the solution was filtered by a 0.22 micron filter. The filtered solution was kept for 3 hours at 18-20° C. In the next step, the solution was added and filled into 100 glass vials (each 50 ml-size vial was filled with about 14 ml of the solution) and lyophilized to give white solids.

The lyophilized solid product was analyzed for its residual solvents. The result for the residual solvents is shown as following: ethanol (0.18%), t-butanol (2.27%) and water (1.0%).

One vial of the lyophilized product was dissolved in 40 ml of 0.9% saline and measured for pH value. The pH value of the reconstituted solution is 5.67.

### Example 15: Liquid Composition Comprising Docetaxel, Human Serum Albumin (HSA), Amino Acids, and Sugar Alcohol using t-butanol and Ethanol as Solvents

Experiment A: Docetaxel (10.1 mg) was dissolved in 0.25 ml of ethanol. 20 mg of aspartic acid was dissolved in 5 ml of water, and then 200 mg of mannitol was added and dissolved in this aqueous solution. Then 5 ml of 20% Human Albumin solution was added into the aqueous solution of aspartic acid and mannitol with rapid stirring at 8-10° C., and followed by adding 3.0 ml of t-butanol. Then the previously prepared docetaxel (10.1 mg) solution in ethanol was added dropwise into this aqueous solution. The container used for preparing the docetaxel ethanol solution was rinsed with 0.25 ml of ethanol, and this ethanol solution was also added into the product solution prepared. Then the product solution was kept at 8-10° C., and the clarity of the solution was checked in different time points. The result was shown in Table 13.

Experiment B: Docetaxel (10.0 mg) was dissolved in 0.25 ml of ethanol. 20 mg of aspartic acid was dissolved in 5 ml of water, and then 200 mg of mannitol was added and dissolved in this aqueous solution. Then 5 ml of 20% Human Albumin solution was added into the aqueous solution of aspartic acid and mannitol with rapid stirring at 8-10° C., and followed by adding 2.5 ml of t-butanol. Then the previously prepared docetaxel (10.0 mg) solution in ethanol was added dropwise into this aqueous solution. The container used for preparing the docetaxel ethanol solution was rinsed with 0.25 ml of ethanol, and this ethanol solution was also added into the product solution prepared. Then the product solution was kept at 8-10° C., and the clarity of the solution was checked in different time points. The result was shown in Table 13.

Experiment C: Docetaxel (10.0 mg) was dissolved in 0.25 ml of ethanol. 20 mg of aspartic acid was dissolved in 5 ml of water, and then 200 mg of mannitol was added and dissolved in this aqueous solution. Then 5 ml of 20% Human Albumin solution was added into the aqueous solution of aspartic acid and mannitol with rapid stirring at 8-10° C., and followed by adding 2.0 ml of t-butanol. Then the previously prepared docetaxel (10.0 mg) solution in ethanol was added dropwise into this aqueous solution. The container used for preparing the docetaxel ethanol solution was rinsed with 0.25 ml of ethanol, and this ethanol solution was added into the product solution prepared. Then the product solution was kept at 8-10° C., and the clarity of the solution was checked in different time points. The result was shown in Table 13.

Experiment D: Docetaxel (10.2mg) was dissolved in 0.25 ml of ethanol. 20 mg of aspartic acid was dissolved in 5 ml of water, and then 200 mg of mannitol was added and dissolved in this aqueous solution. Then 5 ml of 20% Human Albumin solution was added into the aqueous solution of aspartic acid and mannitol with rapid stirring at 8-10° C., and followed by adding 1.0 ml of t-butanol. Then the previously prepared docetaxel (10.2 mg) solution in ethanol was added dropwise into this aqueous solution. The container with used for preparing the docetaxel ethanol solution was rinsed with 0.25 ml of ethanol, and this ethanol solution was also added into the product solution prepared. Then the product solution was kept at 8-10° C., and the clarity of the solution was checked in different time points. The result was shown in Table 13.

TABLE 13
The clarity of the product solution in different time points

| Time | 0 hour | 1 hour | 1.5 hour | 2 hour |
|---|---|---|---|---|
| Experiment A | clear | clear | clear | turbid |
| Experiment B | clear | turbid | — | — |
| Experiment C | turbid | — | — | — |
| Experiment D | turbid | — | — | — |

### Example 16: Liquid Composition Comprising Docetaxel and Human Serum Albumin (HSA) using t-butanol and Ethanol as Solvents

Experiment E: Docetaxel (9.9mg) was dissolved in 0.25 ml of ethanol. 5 ml of 20% Human Albumin solution was added into a 50 ml round-bottom flask with 5 ml of water with rapid stirring at 8-10° C., and followed by adding 3.0 ml of t-butanol. Then the previously prepared docetaxel (9.9 mg) solution in ethanol was added dropwise into this aqueous solution. The container used for preparing the docetaxel ethanol solution was rinsed with 0.25 ml of ethanol, and this ethanol solution was also added into the product solution prepared. Then the product solution was kept at 8-12° C., and the clarity of the solution was checked in different time points. The result was shown in Table 14.

Experiment F: Docetaxel (10.1 mg) was dissolved in 0.25 ml of ethanol. 5 ml of 20% Human Albumin solution was added into a 50 ml round-bottom flask with 5 ml of water with rapid stirring at 8-10° C., and followed by adding 2.5 ml of t-butanol. Then the previously prepared docetaxel (10.1 mg) solution in ethanol was added dropwise into this aqueous solution. The container used for preparing the docetaxel ethanol solution was rinsed with 0.25 ml of ethanol, and this ethanol solution was also added into the product solution prepared. Then the product solution was kept at 8-12° C., and the clarity of the solution was checked in different time points. The result was shown in Table 14.

Experiment G: Docetaxel (10.0 mg) was dissolved in 0.25 ml of ethanol. 5 ml of 20% Human Albumin solution was added into a 50 ml round-bottom flask with 5 ml of water with rapid stirring at 8-10° C., and followed by adding 2.0 ml of t-butanol. Then the previously prepared docetaxel (10.0 mg) solution in ethanol was added dropwise into this aqueous solution. The container used for preparing the docetaxel ethanol solution was rinsed with 0.25 ml of ethanol, and this ethanol solution was also added into the product solution prepared. Then the product solution was kept at 8-12° C., and the clarity of the solution was checked in different time points. The result was shown in Table 14.

Experiment H: Docetaxel (10.1mg) was dissolved in 0.25 ml of ethanol. 5 ml of 20% Human Albumin solution was added into a 50 ml round-bottom flask with 5 ml of water with rapid stirring at 8-10° C., and followed by adding 1.0 ml of t-butanol. Then the previously prepared docetaxel (10.1 mg) solution in ethanol was added dropwise into this aqueous solution. The container used for preparing the docetaxel ethanol solution was rinsed with 0.25 ml of ethanol, and this ethanol solution was also added into the product solution prepared. Then the product solution was kept at 8-12° C., and the clarity of the solution was checked in different time points. The result was shown in Table 14.

TABLE 14
The clarity of the product solution in different time points

| Time | 0 hour | 0.5 hour | 1 hour |
|---|---|---|---|
| Experiment E | clear | clear | turbid |
| Experiment F | clear | turbid | turbid |
| Experiment G | turbid | turbid | turbid |
| Experiment H | turbid | turbid | turbid |

### Example 17: Liquid Composition Comprising Docetaxel, Human Serum Albumin (HSA), Amino Acids, and Sugar Alcohol using t-butanol and Ethanol as Solvents

Experiment 1: Docetaxel (9.9mg) was dissolved in 0.5 ml of ethanol. 20.2 mg of aspartic acid was dissolved in 5 ml of water, and then 201.7 mg of mannitol was added and dissolved in this aqueous solution. Then 5 ml of 20% Human Albumin solution was added into the aqueous solution of aspartic acid and mannitol with rapid stirring at 8-10° C., and followed by adding a mixed solution of 3.0 ml of t-butanol and 0.2 ml ethanol. Then the previously prepared docetaxel (9.9 mg) solution in ethanol was added dropwise into this aqueous solution. The container used for preparing the docetaxel ethanol solution was rinsed with 0.3 ml of ethanol, and this ethanol solution was also added into the product solution prepared. Then the product solution was kept at 8-10° C., and the clarity of the solution was checked in different time points. The result was shown in Table 15.

Experiment 2: Docetaxel (10.1 mg) was dissolved in 0.5 ml of ethanol. 20.2 mg of aspartic acid was dissolved in 5 ml of water, and then 199.2 mg of mannitol was added and dissolved in this aqueous solution. Then 5 ml of 20% Human Albumin solution was added into the aqueous solution of aspartic acid and mannitol with rapid stirring at 8-10° C., and followed by adding a mixed solution of 2.0 ml of t-butanol and 0.2 ml ethanol, and additional 0.8 ml of ethanol. Then the previously prepared docetaxel (10.1 mg) solution in ethanol was added dropwise into this aqueous solution. The container used for preparing the docetaxel ethanol solution was rinsed with 0.5 ml of ethanol, and this ethanol solution was also added into the product solution prepared. Then the product solution was kept at 8-12° C., and the clarity of the solution was checked in different time points. The result was shown in Table 15.

TABLE 15
The clarity of the product solution in different time points

| Time | 0 hour | 1 hour | 2 hour | 3 hour |
|---|---|---|---|---|
| Experiment 1 | clear | clear | clear | clear |
| Experiment 2 | clear | clear | turbid | turbid |

### Example 18:Liquid Composition Comprising Docetaxel and Human Serum Albumin (HSA) using t-butanol and Ethanol as Solvents

Experiment 3: Docetaxel (10.2 mg) was dissolved in 0.5 ml of ethanol. 5 ml of 20% Human Albumin solution was added into a 50 ml round-bottom flask with 5 ml of water with rapid stirring at 8-10° C., and followed by adding a mixed solution of 3.0 ml of t-butanol and 0.2 ml ethanol. Then the previously prepared docetaxel (10.2 mg) solution in ethanol was added dropwise into this aqueous solution. The container used for preparing the docetaxel ethanol solution was rinsed with 0.3 ml of ethanol, and this ethanol solution was also added into the product solution prepared. Then the product solution was kept at 8-10° C., and the clarity of the solution was checked in different time points. The result was shown in Table 16.

Experiment 4: Docetaxel (10.0 mg) was dissolved in 0.5 ml of ethanol. 5 ml of 20% Human Albumin solution was added into a 50 ml round-bottom flask with 5 ml of water with rapid stirring at 8-10° C., and followed by adding a mixed solution of 2.0 ml of t-butanol and 0.2 ml ethanol, and additional 0.8 ml of ethanol. Then the previously prepared docetaxel (10.1 mg) solution in ethanol was added dropwise into this aqueous solution. The container used for preparing the docetaxel ethanol solution was rinsed with 0.5 ml of ethanol, and this ethanol solution was also added into the product solution prepared. Then the product solution was kept at 8-12° C., and the clarity of the solution was checked in different time points. The result was shown in Table 16.

TABLE 16
The clarity of the product solution in different time points

| Time | 0 hour | 1 hour | 2 hour | 3 hour |
|---|---|---|---|---|
| Experiment 3 | clear | clear | clear | clear |
| Experiment 4 | clear | clear | clear | turbid |

### Example 19: Liquid Composition Comprising Docetaxel, Human Serum Albumin (HSA), Amino Acids, and Sugar Alcohol using t-butanol and Ethanol as Solvents

Experiment 5: Docetaxel (10.1 mg) was dissolved in 0.5 ml of ethanol. 19.8 mg of aspartic acid was dissolved in 5 ml of water, and then 200.2 mg of mannitol was added and dissolved in this aqueous solution. Then 5 ml of 20% Human Albumin solution was added into the aqueous solution of aspartic acid and mannitol with rapid stirring at 8-10° C., and followed by adding a mixed solution of 0.5 ml of t-butanol and 0.5 ml ethanol, and additional 2 ml ethanol. Then the previously prepared docetaxel (10.1 mg) solution in ethanol was added dropwise into this aqueous solution. The container used for preparing the docetaxel ethanol solution was rinsed with 0.5 ml of ethanol, and this ethanol solution was also added into the product solution prepared. Then the product solution was kept at 8-10° C., and the clarity of the solution was checked in different time points. The result was shown in Table 17.

Experiment 6: Docetaxel (10.1 mg) was dissolved in 0.5 ml of ethanol. 19.9 mg of aspartic acid was dissolved in 5 ml of water, and then 200.3 mg of mannitol was added and dissolved in this aqueous solution. Then 5 ml of 20% Human Albumin solution was added into the aqueous solution of aspartic acid and mannitol with rapid stirring at 8-10° C., and followed by adding a mixed solution of 0.2 ml of t-butanol and 0.5 ml ethanol, and additional 2.3 ml of ethanol. Then the previously prepared docetaxel (10.1 mg) solution in ethanol was added dropwise into this aqueous solution. The container used for preparing the docetaxel ethanol solution was rinsed with 0.5 ml of ethanol, and this ethanol solution was also added into the product solution prepared. Then the product solution was kept at 8-12° C., and the clarity of the solution was checked in different time points. The result was shown in Table 17.

Experiment 7: Docetaxel (10.1 mg) was dissolved in 0.5 ml of ethanol. 19.9 mg of aspartic acid was dissolved in 5 ml of water, and then 200.6 mg of mannitol was added and dissolved in this aqueous solution. Then 5 ml of 20% Human Albumin solution was added into the aqueous solution of aspartic acid and mannitol with rapid stirring at 8-10° C., and followed by adding a mixed solution of 1.0 ml of t-butanol and 0.5 ml ethanol, and additional 1.5 ml ethanol. Then the previously prepared docetaxel (10.1 mg) solution in ethanol was added dropwise into this aqueous solution. The container used for preparing the docetaxel ethanol solution was rinsed with 0.5 ml of ethanol, and this ethanol solution was also added into the product solution prepared. Then the product solution was kept at 8-10° C., and the clarity of the solution was checked in different time points. The result was shown in Table 17.

TABLE 17
The clarity of the product solution in different time points

| Time | 0 minute | 5 minute | 10 minute | 15 minute |
|---|---|---|---|---|
| Experiment 5 | clear | NA | turbid | NA |
| Experiment 6 | clear | NA | turbid | NA |
| Experiment 7 | clear | clear | NA | turbid |

### Example 20: Liquid Composition Comprising Docetaxel and Human Serum Albumin (HSA) using t-butanol and Ethanol as Solvents

Experiment 8: Docetaxel (10.1 mg) was dissolved in 0.5 ml of ethanol. 5 ml of 20% Human Albumin solution was added into a 50 ml round-bottom flask with 5 ml of water with rapid stirring at 8-10° C., and followed by adding a mixed solution of 0.5 ml of t-butanol and 0.5 ml ethanol, and additional 2 ml ethanol. Then the previously prepared docetaxel (10.1 mg) solution in ethanol was added dropwise into this aqueous solution. The container used for preparing the docetaxel ethanol solution was rinsed with 0.5 ml of ethanol, and this ethanol solution was also added into the product solution prepared. Then the product solution was kept at 8-12° C., and the clarity of the solution was checked in different time points. The result was shown in Table 18.

Experiment 9: Docetaxel (10.1 mg) was dissolved in 0.5 ml of ethanol. 5 ml of 20% Human Albumin solution was added into a 50 ml round-bottom flask with 5 ml of water with rapid stirring at 8-10° C., and followed by adding a mixed solution of 0.2 ml of t-butanol and 0.5 ml ethanol, and additional 2.3 ml ethanol. Then the previously prepared docetaxel (10.1 mg) solution in ethanol was added dropwise into this aqueous solution. The container used for preparing the docetaxel ethanol solution was rinsed with 0.5 ml of ethanol, and this ethanol solution was also added into the product solution prepared. Then the product solution was kept at 8-12° C., and the clarity of the solution was checked in different time points. The result was shown in Table 18.

Experiment 10: Docetaxel (10.0 mg) was dissolved in 0.5 ml of ethanol. 5 ml of 20% Human Albumin solution was added into a 50 ml round-bottom flask with 5 ml of water with rapid stirring at 8-10° C., and followed by adding a mixed solution of 1.0 ml of t-butanol and 0.5 ml ethanol, and additional 1.5 ml ethanol. Then the previously prepared docetaxel (10.0 mg) solution in ethanol was added dropwise into this aqueous solution. The container used for preparing the docetaxel ethanol solution was rinsed with 0.5 ml of ethanol, and this ethanol solution was also added into the product solution prepared. Then the product solution was kept at 8-12° C., and the clarity of the solution was checked in different time points. The result was shown in Table 18.

TABLE 18
The clarity of the product solution in different time points

| Time | 0 minute | 10 minute |
|---|---|---|
| Experiment 8 | clear | turbid |
| Experiment 9 | clear | turbid |
| Experiment 10 | clear | turbid |

### Example 21: Liquid Composition Comprising Docetaxel and Human Serum Albumin (HSA) using t-butanol and Ethanol as Solvents

Experiment 11: Docetaxel (9.9 mg) was dissolved in 0.2 ml of ethanol and 3.8 ml of t-butanol. 5 ml of 20% Human Albumin solution was added into a 50 ml round-bottom flask with 5 ml of water. Then the previously prepared docetaxel (9.9 mg) solution in ethanol and t-butanol was added dropwise into this aqueous solution with rapid stirring at 8-10° C. A clear yellow product solution was obtained. Then the product solution was kept at 8-12° C., and the clarity of the solution was checked in different time points. The result was shown in Table 19.

Experiment 12: Docetaxel (9.9 mg) was dissolved in 0.2 ml of ethanol and 3.8 ml of t-butanol. 20.4 mg of aspartic acid was dissolved in 5 ml of water and cooled to 25° C., and then 200.1 mg of mannitol was added and dissolved in this aqueous solution. Then 5 ml of 20% Human Albumin solution was added into the aqueous solution of aspartic acid and mannitol. Then the previously prepared docetaxel (9.9 mg) solution in ethanol and t-butanol was added dropwise into this aqueous solution with rapid stirring at 8-10° C. A clear yellow product solution was obtained. Then the product solution was kept at 8-12° C., and the clarity of the solution was checked in different time points. The result was shown in Table 19.

TABLE 19
The clarity of the product solution in different time points

| Time | 0 hour | 1 hour | 3 hour | 4 hour |
|---|---|---|---|---|
| Experiment 11 | clear | clear | clear | clear |
| Experiment 12 | clear | clear | clear | clear |

### Example 22: Liquid Composition Comprising Docetaxel and Human Serum Albumin (HSA) using t-butanol and Ethanol as Solvents

Docetaxel (25.1 mg) was dissolved in 0.25 ml of ethanol and 9.75 ml of t-butanol in a 20 ml vial, which was used in the two following experiments.

Experiment 13: 5 ml of 20% Human Albumin solution was added into a 50 ml round-bottom flask with 5 ml of water at 6-8° C. Then 4 m of the previously prepared docetaxel solution in ethanol and t-butanol was added dropwise into this aqueous solution with stirring. A clear yellow product solution was obtained. Then the product solution was kept at 8-12° C., and the clarity of the solution was checked in different time points. The result was shown in Table 20.

Experiment 14: 20 mg of aspartic acid as dissolved in 5 ml of water and cooled down, and then 198.9 mg of mannitol was added and dissolved in this aqueous solution. Then 5 ml of 20% Human Albumin solution was added into the aqueous solution of aspartic acid and mannitol with rapid stirring at 8-10° C. Then 4 m of the previously prepared docetaxel solution in ethanol and t-butanol was added dropwise into this aqueous solution. A clear yellow product solution was obtained. Then the product solution was kept at 8-12° C., and the clarity of the solution was checked in different time points. The result was shown in Table 20.

TABLE 20
The clarity of the product solution in different time points

| Time | 0 hour | 1 hour | 3 hour | 4 hour |
|---|---|---|---|---|
| Experiment 13 | clear | clear | clear | clear |
| Experiment 14 | clear | clear | clear | clear |

### Example 23: pH Values and HPLC Studies of Liquid Composition Comprising Docetaxel, Human Serum Albumin (HSA), Amino Acids, and Sugar Alcohol using t-butanol and Ethanol as Solvents

Docetaxel (40.2 mg) was dissolved in 2 ml of ethanol. 14 ml of t-butanol was added into this solution and mixed well.

80.4 mg of aspartic acid was dissolved in 20 ml of water and cooled down to room temperature, and then 800.8 mg of mannitol was added and dissolved in this aqueous solution. Then 20 ml of 20% Human Albumin solution was addeded into the aqueous solution of aspartic acid and mannitol with rapid stirring at 8-10° C. Then the previously prepared docetaxel (40.2 mg) solution in ethanol and t-butanol was added dropwise into this aqueous solution with rapid stirring at 8-10° C. A clear yellow solution was obtained. 6 ml of the clear product solution was measured for pH value. The pH value of the clear solution is 6.02 (4 measurements: 6.04, 6.02, and 6.01).

The remaining product solution was kept at 8-10° C. At 0 hour (right after the preparation of the product solution), to 2 ml of the product solution before the filtration was added 5 ml of acetonitrile. The mixture was centrifuged at 3,500 g for 4 minutes. The supernatant was removed and collected followed by injection on HPLC to check the amount of docetaxel. To 2 ml of the product solution after filtered by a 0.22 micron filter was added 5 ml of acetonitrile. The mixture was centrifuged at 3,500 g for 4 minutes. The supernatant was removed and collected followed by injection on HPLC to check the amount of docetaxel. At 0 hour, the Docetaxel amount in the solution after the filtration was about 99.85% of the Docetaxel amount in the solution before the filtration.

The same experiments were repeated at 1 hour and 2 hour. At 1 hour, the Docetaxel amount in the solution after the filtration was about 99.39% of the Docetaxel amount in the solution before the filtration. At 2 hour, the Docetaxel amount in the solution after the filtration was about 100.4% of the Docetaxel amount in the solution before the filtration.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising Docetaxel, human serum albumin, and one or more amino acids selected from aspartic acid, glutamic acid, and cysteine, wherein the human serum albumin and the Docetaxel in the composition have a ratio by weight of no less than 60:1, and wherein the amino acid and the Docetaxel in the composition have a ratio by weight of no less than 0.2:1.

2. The composition of claim 1, wherein the Docetaxel and the human serum albumin in the composition are in a ratio by weight from about 1:80 to about 1:150.

3. The composition of claim 1, wherein the Docetaxel and the human serum albumin in the composition are in a ratio by weight of about 1:100.

4. The composition of claim 1, wherein the amino acid in the composition is aspartic acid.

5. The composition of claim 1, wherein the amino acid and the Docetaxel in the composition have a ratio by weight of about 2:1.

6. The composition of any one of claim 1, wherein the composition comprises a sugar alcohol or a sugar, and wherein the sugar alcohol or sugar and the Docetaxel in the composition have a ratio by weight of no less than about 5:1 .

7. The composition of claim 1, wherein 7-Epi-docetaxel and the Docetaxel in the composition have a ratio by weight of no more than 1:100.

8. The composition of claim 1, wherein 7-Epi-docetaxel and the Docetaxel in the composition have a ratio by weight of no more than 1:200.

9. The composition of claim 1, wherein the composition is a solid formulation.

10. The composition of claim 1, wherein the composition is an aqueous formulation.

11. The composition of claims 10, wherein the aqueous formulation is a clear aqueous solution.

12. The composition of claim 10, wherein the aqueous formulation has pH value from about 4 to about 8.

13. The composition of claim 10, wherein the aqueous formulation has pH value from about 5 to about 7.

14. A pharmaceutical composition comprising the composition of claim 1, and a pharmaceutically acceptable carrier.

15. A method of treating a cancer, the method comprising the step of administering to a subject in need thereof a therapeutically effective amount of a pharmaceutical composition of claim 14.

16. The method of claim 15, wherein the cancer is selected from the group consisting of breast cancer, non-small cell lung cancer, prostate cancer, gastric cancer, head and neck cancer, ovarian cancer, pancreatic cancer, and Kaposi's sarcoma.

17. A composition comprising Docetaxel, human serum albumin, and a sugar alcohol or a sugar, wherein the human serum albumin and the Docetaxel in the composition have a ratio by weight of no less than 60:1.

18. A composition comprising Docetaxel and human serum albumin, wherein 7-Epi-docetaxel and the Docetaxel in the composition have a ratio by weight of no more than 1:200, and wherein the human serum albumin and the Docetaxel in the composition have a ratio by weight of no less than 60:1.

19. A liquid composition comprising Docetaxel and human serum albumin, wherein the human serum albumin and the Docetaxel in the composition have a ratio by weight of no less than 60:1, wherein the composition comprises water, t-butanol, and ethanol as solvents, wherein the pH of the composition is from about 4 to about 8, and wherein t-butanol and ethanol in the composition are in a ratio from about 1:20 to about 100:1 (v/v).

20. (canceled)
